(19) United States
(12) Patent Application Publication
(10) Pub. No.: US 2014/0234926 A1
(43) Pub. Date: Aug. 21, 2014

(54) RECOMBINANT ANAEROBIC ACETOGENIC BACTERIA FOR PRODUCTION OF ISOPRENE AND/OR INDUSTRIAL BIO-PRODUCTS USING SYNTHESIS GAS

(71) Applicants: E I Du Pont De Nemours And Company, Wilmington, DE (US); The Goodyear Tire And Rubber Company, Akron, OH (US); Danisco US Inc, Palo Alto, CA (US)

(72) Inventors: ZACHARY Q. BECK, Palo Alto, CA (US); Marguerite A. Cervin, Palo Alto, CA (US); Gopal K. Chotani, Palo Alto, CA (US); Bruce A. Diner, Chadds Ford, PA (US); Janine Fan, Hockessin, DE (US); Caroline M. Peres, Palo Alto, CA (US); Karl J. Sanford, Palo Alto, CA (US); Miles C. Scotcher, Palo Alto, CA (US); Derek H. Wells, Palo Alto, CA (US); Gregory M. Whited, Palo Alto, CA (US)

(73) Assignees: E I Du Pont De Nemours And Company, Wilmington, DE (US); The Goodyear Tire And Rubber Company, Akron, OH (US); Danisco US Inc, Palo Alto, CA (US)

(21) Appl. No.: 14/091,449

(22) Filed: Nov. 27, 2013

Related U.S. Application Data
(63) Continuation-in-part of application No. PCT/US2013/043804, filed on May 31, 2013.
(60) Provisional application No. 61/654,737, filed on Jun. 1, 2012.
(60) Provisional application No. 61/654,736, filed on Jun. 1, 2012.
(60) Provisional application No. 61/654,765, filed on Jun. 1, 2012.

(51) Int. Cl. C12P 7/42; C12P 5/00

(52) U.S. Cl. CPC C12P 7/42; C12P 5/007
USPC 435/146; 435/252.3; 435/167

(57) ABSTRACT

This invention provides for recombinant anaerobic acetogenic bacterial cells having one or more nucleic acids whereby isoprene, mevalonate and/or other industrial bio-products are produced in a substantially oxygen-free culture condition using synthesis gas as energy and/or carbon source.

### CROSS-REFERENCE TO RELATED APPLICATIONS

This application is a continuation in part of PCT/US13/43804, filed on May 31, 2013 which claims priority to: (1) U.S. Provisional Application No. 61/654,737, filed on Jun. 1, 2012, (2) U.S. Provisional Application No. 61/654,736, filed on Jun. 1, 2012, and (3) U.S. Provisional Application No. 61/654,765, filed on Jun. 1, 2012, each now expired where the entire contents of all three applications are hereby incorporated by reference in their entirety.

### FIELD OF THE INVENTION

The invention is in the field of anaerobic microorganisms which have been engineered to produce isoprene and/or industrial bio-products under substantially oxygen-free conditions using synthesis gas, carbohydrate, and/or carbohydrate and hydrogen as an energy and/or carbon source.

### BACKGROUND OF THE INVENTION

Much emphasis is put on designing energy-efficient processes to convert renewables to biofuels, biopolymers and biochemicals. Isoprene (2-methyl-1,3-butadiene) is the critical starting material for a variety of synthetic polymers, most notably synthetic rubbers. Isoprene is naturally produced by a variety of microbial, plant, and animal species. In particular, two pathways have been identified for the biosynthesis of isoprene: the mevalonate (MVA) pathway and the non-mevalonate (DXP) pathway. However, the yield of isoprene from naturally-occurring organisms is commercially unattractive. About 800,000 tons per year of cis-polyisoprene are produced from the polymerization of isoprene; most of this polyisoprene is used in the tire and rubber industry. A dependable supply of isoprene is needed in billion(s) of pounds with low manufacturing costs. Like all monomers, isoprene has to be highly pure for making polymers. Isoprene is also copolymerized for use as a synthetic elastomer in other products such as footwear, mechanical products, medical products, sporting goods, and latex.

Currently, the tire and rubber industry is based on the use of natural and synthetic rubber. Natural rubber is obtained from the milky juice of rubber trees or plants found in the rainforests of Africa. Synthetic rubber is based primarily on butadiene polymers. For these polymers, butadiene is obtained as a co-product from ethylene and propylene manufacture.

While isoprene can be obtained by fractionating petroleum, the purification of this material is expensive and time-consuming. Petroleum cracking of the C5 stream of hydrocarbons produces only about 15% isoprene. Thus, more economical methods for producing isoprene are needed. In particular, methods that produce isoprene at rates, titers, and purity that are sufficient to meet the demands of a robust commercial process are desirable. Also desired are systems for producing isoprene from inexpensive starting materials.

Microorganisms provide a means for converting renewable materials to biofuels, biopolymers and biochemicals in large quantities, good purities, and low manufacturing costs. Obligate anaerobic bacteria such as Clostridium carboxydivorans, Clostridium ljungdahlii, Clostridium autoethanogenum, Peptostreptococcus productus, and Eurobacterium limosum naturally produce bioproducts such as ethanol, butanol, methane, and hydrogen via fermentation. By using synthesis gas as a carbon source and adjusting the growth conditions and reactor design, the yields of these bioproducts has been increased beyond the natural yields to produce commercially relevant quantities of bioethanol, biobutanol, etc. (see, e.g., Hurst et al., Biochemical Engineering Journal article in press (2009); Cotter et al., Enzyme and Microbial Technology 44:281-288 (2009); Henstra et al., Current Opinion in Biotechnology 18: 200-206 (2007); Misoph et al., Journal of Bacteriology 178(1): 3140-3145 (1996), Change et al., Process Biochemistry 37:411-421 (2001); and Ahmed et al., Biotechnology and Bioengineering 97(5): 1080-1086 (2006)).

A few anaerobic bacteria, such as Bacillus cereus 6A1 and Bacillus lichenformis 5A24, have been found to naturally produce isoprene in small quantities (see, e.g., U.S. Pat. No. 5,849,870). Generally, however, anaerobic bacteria do not naturally produce isoprene in commercially relevant quantities. Anaerobic bacteria have been engineered to convert CO, CO₂, and/or H₂, the primary components of synthesis gas, to Acetyl-CoA (see, e.g., WO2009/094485). However, anaerobic bacteria have not previously been engineered to convert syngas to isoprene. Accordingly, there remains a need for engineered anaerobic bacteria to produce isoprene in a system that is substantially free of oxygen and in the presence of synthesis gas, and methods for making and using such microorganisms to produce industrial bio-products.

Throughout the specification, various publications (including sequences), patents, and patent applications are disclosed. All of these are hereby incorporated by reference in their entirety for all purposes.

### BRIEF SUMMARY OF THE INVENTION

The present invention provides acetogenic bacterial cells that grow anaerobically on synthesis gas and have been genetically engineered to produce isoprene.

Accordingly, the invention provides a recombinant obligate anaerobic bacterial acetogen cells comprising a heterologous nucleic acid encoding an isoprene synthase polypeptide and a heterologous nucleic acid encoding an isopentenyl diphosphate isomerase polypeptide, which are operably linked either separately or in an operon to a promoter that is functional in the cells, wherein the cells produce more isoprene when grown in substantially oxygen-free culture conditions comprising synthesis gas as compared to the cells without the heterologous nucleic acids.

In some embodiments the cells further comprise one or more introduced nucleic acids encoding one or more DXP pathway polypeptide(s), wherein the DXP pathway polypeptide(s) have increased expression as compared to the cells without the introduced nucleic acids encoding DXP pathway polypeptide(s).

In some embodiments the cells further comprise introduced nucleic acids encoding MVA pathway polypeptide(s) having acetyl-CoA acetyltransferase, 3-hydroxy-3-methylglutaryl-CoA (HMG-CoA) reductase, 3-hydroxy-3-methylglutaryl-CoA (HMG-CoA) synthase, mevalonate kinase, phosphomevalonate kinase, and diphosphomevalonate decarboxylase.

In a further embodiment, the invention provides a method of producing isoprene comprising culturing any of the cells described above under suitable conditions for the production of isoprene, wherein the culture conditions are substantially oxygen-free and comprise synthesis gas as energy and/or carbon source.

In yet another embodiment, the invention provides recombinant obligate anaerobic bacterial acetogen cells comprising at least one heterologous nucleic acid encoding at least two polypeptides having acetyl-CoA acetyltransferase 3-hydroxy-3-methylglutaryl-CoA (HMG-CoA) reductase, and 3-hydroxy-3-methylglutaryl-CoA (HMG-CoA) synthase activities, which are operably linked either separately or in an operon to a promoter that is active in the cells, wherein the cells produce mevalonate when grown in substantially oxygen-free culture conditions comprising synthesis gas and/or carbon source.

In a further embodiment, the invention provides a method of producing mevalonate comprising culturing cells above under suitable conditions for the production of mevalonate, wherein the culture conditions are substantially oxygen-free and comprise synthesis gas as energy and/or carbon source.

It is to be understood that one, some, or all of the properties of the various aspects described herein may be combined to form other aspects of the present invention. These and other aspects of the invention will become apparent to one of skill in the art.

### BRIEF DESCRIPTION OF THE FIGURES AND SEQUENCE DESCRIPTIONS

The various embodiments of the invention can be more fully understood from the following detailed description, the figures, and the accompanying sequence descriptions, which form a part of this application.

FIG. 1 illustrates the classical and modified MVA pathways. 1, acetyl-CoA acetyltransferase (AACT); 2, HMG-CoA synthase (HMGS); 3, HMG-CoA reductase (HMGR); 4, mevalonate kinase (MVK); 5, phosphomevalonate kinase (PMK); 6, diphosphomevalonate decarboxylase (MVD or DPMDC); 7, isopentenyl diphosphate isomerase (IDI); 8, phosphomevalonate decarboxylase (PMDC); 9, isopentenyl phosphate kinase (IPK). The classical MVA pathway proceeds from reaction 1 through reaction 7 via reactions 5 and 6, while a modified MVA pathway goes through reactions 8 and 9. P and PP in the structural formula are phosphate and pyrophosphate, respectively. This figure was taken from Koga and Morii, Microbiology and Mol. Biology Reviews, 71:97-120, 2007, which is incorporated by reference in its entirety, particularly with respect to nucleic acids and polypeptides of the modified MVA pathway. The modified MVA pathway is present, for example, in some archaeal organisms, such as Methanosarcina mazei.

FIG. 2 shows a plot of moles of syngas components (CO₂, CO, H₂, and H₂O) reacting compared to moles of isoprene, CO₂ and H₂O produced, according to Equation 7, for values of n from 0 to 14. Negative numbers in the equation are plotted as zero.

FIG. 3 shows various pathways in obligate anaerobes expressing heterologous isoprene synthase and a heterologous isopentenyl diphosphate isomerase.

FIG. 4 shows a schematic representation of an obligate anaerobe engineered with mvaE and mvaS to express upper MVA pathway.

FIG. 5 shows a schematic representation of expressing lower MVA pathway in an obligate anaerobe including expressing (a) a heterologous MVK polypeptide, (b) a heterologous PMK polypeptide, and (c) a heterologous MVD polypeptide in the cells expressing heterologous IDI polypeptide and heterologous IspS polypeptide for the purpose of increasing isoprene production.

FIG. 6 shows a schematic representation of expressing entire MVA pathway in an obligate anaerobe by introducing mvaE and mvaS in the cells expressing (a) a heterologous MVK polypeptide, (b) a heterologous PMK polypeptide, (c) a heterologous MVD polypeptide, (d) a heterologous IDI polypeptide, and (e) a heterologous IspS polypeptide for the purpose of increasing isoprene production.

FIG. 7 shows a schematic representation of an obligate anaerobe expressing (a) a heterologous IspS polypeptide, (b) a heterologous DXS polypeptide, and (c) a heterologous IDI polypeptide to increase DXP pathway flux and isoprene production.

FIG. 8 shows pathway, physiology, and yield calculations for simultaneous heterotrophic and autotrophic biosynthesis of isoprene from carbohydrates and hydrogen (or syngas).

FIG. 9 shows a schematic representation of redirecting carbon flux away from acetate by reducing expression of ack and adhE to reduce loss of carbon to side products. The arrows next to Ack or AdhE used in the production of acetate and ethanol, respectively, indicate a reduction of activity or enzyme expression for pathways leading to fermentation products such as acetate, ethanol, or any other alcohol, or carbon containing end product. The purpose is to maximize carbon channeling to isoprene via genetic manipulation.

FIG. 10 shows the industrial products that can be produced from syngas via cellular pathways.

FIG. 11 shows the microbial fuels that can be produced from syngas via cellular pathways.

FIG. 12 shows the plasmid map for pMCS278.

FIG. 13 shows the plasmid map for pJF100.

FIG. 14 shows Western blots demonstrating expression of MvaE (14A) and MvaS (14B) in C. ljungdahlii transformants containing pJF100, and in control TV3007 E. coli transformants containing pJF 100.

FIG. 15A-15D show the refractive index detected (RID) HPLC elution profiles between 18 and 21 minutes of acidified fermentation broth (300 μL cell suspension+54 μL 10% H2SO4) of wild-type (FIG. 15A), pMCS201 (FIG. 15B), and pJF100 (FIG. 15C) strains of Clostridium ljungdahlii, showing a comparison of the sealed vial to the equilibrated bottle condition in each case. FIG. 15D shows a comparison of the equilibrated bottle conditions for wild-type C. ljungdahlii (solid line), pJF100-transformed C. ljungdahlii (thick dashed line), and pMCS201-transformed C. ljungdahlii (dotted line).

FIG. 16 shows a plot of growth and mevalonate production for C. ljungdahlii pJF 100 transformant grown on MES-F medium at 37° C. under an atmosphere of either N₂ alone or 2% H₂, 5% CO₂ and 93% N₂.

FIG. 17 shows a comparison of the sealed vial conditions for wild-type C. ljungdahlii (solid line), pJF100-transformed C. ljungdahlii (thick dashed line), and pMCS201-transformed C. ljungdahlii (thin dotted line).

FIG. 18 shows the plasmid map for pDW253.

FIG. 19 shows the plasmid map for pJF100 Fdii.

FIG. 20 shows Western blots demonstrating expression of IspS (20A) and Idi (20B) in the four C. ljungdahlii transformants, pJF100 Fdii #1, plasmid 1, #4 plasmid 1, #9 plasmid 3 and #11 plasmid 3 and in the transformants in E. coli TV3007 generated using plasmids pJF200 #1LD and #2LD.

FIG. 21 shows Western blots demonstrating expression of IspS (21A) and Idi (21B) in the four C. ljungdahlii transformants, pJF100 Fdii N-term His#1&2 and pJF100 Fdii C-term plasmid #1 and 4, and pJf100 Fdii transformants as controls.

FIG. 22A shows a plot comparing growth of pre- and post syngas selection WT C. ljungdahlii on MES-0F medium (without fructose) under a syngas atmosphere. The syngas atmosphere was refreshed at each cell density measurement. FIG. 22B shows a plot of growth curves under syngas of C. ljungdahlii syngas-adapted WT (▪) and pJF100 (▴) strains grown first on MES-0.1F (closed symbols) and then in MES-0F (open symbols, □ and ∘, respectively) after spinning down and resuspending in 5 mL MES-0F medium at the indicated cell densities in 160 mL sealed bottles.

FIG. 23 is a plot showing a growth curve and mevalonate production under a syngas atmosphere for pJF100 first diluted 1:10 in MES-0F+5 μg/mL thiamphenicol and then a second passage after spinning down and resuspending in MES-0F+5 μg/mL thiamphenicol medium after growth to 1.09 OD600 on MES-0.1F+5 μg/mL thiamphenicol under a syngas atmosphere. The mevalonic acid concentrations are the average of the m/z=147 peak and the 147→59 SRM transition (see Table 13).

The following sequences conform with 37 C.F.R. 1.821-1.825 (“Requirements for patent applications Containing Nucleotide Sequences and/or Amino Acid Sequence Disclosures—the Sequence Rules”) and are consistent with World Intellectual Property Organization (WIPO) Standard ST.25 (2009) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is the origin of replication in pCB 102 isolated from a C. butyricum plasmid.

SEQ ID NO:2 is the amino acid sequence of Acetoactyl-CoA-synthase from Streptomyces sp. CL190.

SEQ ID NO:3 is the amino acid sequence of MvaS from E. faecalis with a his-tag.

SEQ ID NO:4 is the amino acid sequence of MvaE from E. faecalis with a his-tag.

SEQ ID NO:5 is the amino acid sequence of IDI from S. cerevisiae with a his-tag.

SEQ ID NO:6 is the amino acid sequence of IspS from P. alba with a his-tag

SEQ ID NO:7 is the coding region for MvaE from E. faecalis.

SEQ ID NO:8 is the amino acid sequence of MvaE from E. faecalis.

SEQ ID NO:9 is the coding region for MvaS from E. faecalis.

SEQ ID NO:10 is the amino acid sequence of MvaS from E. faecalis.

SEQ ID NO:11 is the nucleotide sequence of the pMCS278 plasmid.

SEQ ID NOs:12-17 are oligonucleotide primers.

SEQ ID NO:18 is the nucleotide sequence of the pJF100 plasmid.

SEQ ID NOs:19-22 are oligonucleotide primers.

SEQ ID NO:23 is the nucleotide sequence of the pMCS337 plasmid.

SEQ ID NOs:24-25 are oligonucleotide primers.

SEQ ID NO:26 is the amino acid sequence of the truncated P. alba IspS.

SEQ ID NO:27 is the codon optimized coding region for truncated P. alba IspS.

SEQ ID NO:28 is the codon optimized coding region for S. cerevisiae IDI.

SEQ ID NO:29 is the amino acid sequence of S. cerevisiae IDI.

SEQ ID NOs:30-34 are oligonucleotide primers.

SEQ ID NO:35 is the nucleotide sequence of the pJF200 plasmid.

SEQ ID NOs:36-54 are oligonucleotide primers.

SEQ ID NO:55 is the nucleotide sequence of the pJF100 Fdii plasmid.

SEQ ID NOs:56-61 are oligonucleotide primers.

SEQ ID NO:62 is the origin of replication in pBP1 isolated from a C. botulinum plasmid.

### DETAILED DESCRIPTION OF THE INVENTION

The invention features anaerobic organisms (e.g., microorganisms) capable of making isoprene and other products using synthesis gas (syngas), carbohydrate, and/or carbohydrate and hydrogen, compositions comprising such organisms (e.g., microorganisms), methods of making and using such organisms (e.g., microorganisms) for producing isoprene and/or other desired products. Engineering anaerobic microorganisms to produce isoprene and/or other desired products by fermentation of syngas (or carbohydrate or a combination of carbohydrate and hydrogen) provides a means of producing such products in high yields and good purities via a cost-effective commercializable process. Engineering biochemical pathways from a number of organisms, including plants, into a variety of anaerobic microorganisms, allows for the production of isoprene by fermentation. There are a number of challenges associated with engineering biochemical pathways in anaerobic microorganism for the purpose of producing isoprene, including, for example, heterologous nucleic acids introduced into host anaerobic cells for producing isoprene are degraded and/or not stable in the host anaerobic cells.

The inventors of the present disclosure provide herein, inter alia, that it is possible to produce isoprene by anaerobic fermentation of synthesis gas produced from feedstocks such as biomass (e.g., wood, switch grass, agriculture waste, municipal waste), coal, petroleum, natural gas, rubber tires, and a mixture thereof. In particular, various embodiments (e.g., employment of inducible promoter or constitutive promoter with low expression, or strains in which engineered polypeptides are resistant to degradation) are disclosed for the purpose of making engineered anaerobic cells that are capable of producing high level isoprene.

Accordingly, in one aspect, the invention features novel compositions and methods for engineering an anaerobic microorganism (e.g., obligate anaerobe) to produce isoprene or other products using synthesis gas, carbohydrate, and/or a combination of carbohydrate and hydrogen. In another aspect, the invention features novel methods to engineer a pathway for production of isoprene in microorganisms which naturally grow under oxygen-free conditions on synthesis gas. In another aspect, the invention features anaerobic cells capable of producing isoprene under substantially oxygen-free culture conditions. In another aspect, the invention features methods of producing isoprene from syngas using anaerobic cells. In yet another aspect, the invention features isoprene produced by any of the compositions or methods described herein.

Also provided herein are anaerobic cells (e.g., obligate anaerobic cells or facultative anaerobic cells) comprising one or more heterologous nucleic acids encoding isoprene synthase polypeptide, wherein the cells are capable of producing isoprene in a substantially oxygen-free culture condition comprising carbohydrate and hydrogen as energy and/or carbon source. Any of the anaerobic cells, promoters, the vectors, the isoprene synthase polypeptides, and the methods of making and using thereof provided herein that are used for making isoprene from syngas may be used for making isoprene from carbohydrate and hydrogen.

In other aspects of the invention, anaerobes are used to make industrial products, such as industrial enzymes either alone or with isoprene. Provided herein are anaerobic cells comprising one or more nucleic acids encoding an industrial enzyme, wherein the cells are capable of producing the industrial enzyme in a substantially oxygen-free culture condition comprising synthesis gas as energy and/or carbon source. In some aspects, the one or more nucleic acids encoding an industrial enzyme are heterologous nucleic acids. In some aspects, the one or more nucleic acids encoding an industrial enzyme are endogenous nucleic acids (e.g., extra copies of endogenous nucleic acids). In some aspects, the cells may further comprise one or more heterologous nucleic acids encoding isoprene synthase. Any of the anaerobic cells, promoters, the vectors, the isoprene synthase polypeptides, and the methods of making and using thereof provided herein that are used for making isoprene may be used for making industrial enzyme(s).

### General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Handbook on Clostridia” (P. Dune, ed., 2004), Brock, “Biotechnology: A Textbook of Industrial Microbiology” (Brock, Sinauer Associates, Inc. Second Edition, 1989), “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994), Dictionary of Microbiology and Molecular Biology (Singleton et al., 2nd ed., J. Wiley & Sons, New York, N.Y. 1994), and “Advanced Organic Chemistry Reactions, Mechanisms and Structure” (March, 4th ed., John Wiley & Sons, New York, N.Y. 1992), which provide one skilled in the art with a general guide to many of the terms and methods used in the present disclosure.

Primers, oligonucleotides and polynucleotides employed in the present invention can be generated using standard techniques known in the art.

Unless defined otherwise, technical and scientific terms used herein have the same meaning as commonly understood by one of ordinary skill in the art to which this invention belongs.

### DEFINITIONS

An “anaerobe” is an microbial organism that does not require oxygen for growth. An anaerobe can be an obligate anaerobe, a facultative anaerobe, or an aerotolerant organism.

An “acetogen” is a microbial organism that is able to convert Acetyl-CoA to acetate. Acetogens may be anaerobes or obligate anaerobes.

A “carbohydrate” is defined herein as a compound that consists only of carbon, hydrogen, and oxygen atoms, in any ratio. “Carbohydrates” include, but are not limited to sugars or sugar alcohols. Carbohydrates include monosaccharides (e.g., glucose, fructose, galactose, xylose, arabinose, or ribose), sugar derivatives (e.g., sorbitol, glycerol, galacturonic acid, rhamnose, xylitol), disaccharides (e.g., sucrose, cellobiose, maltose, or lactose), oligosaccharides (e.g., xylooligomers, cellodextrins, or maltodextrins), and polysaccharides (e.g., xylan, cellulose, starch, mannan, alginate, or pectin).

“C5 carbohydrate” refers to any carbohydrate, without limitation, that has five (5) carbon atoms. C5 carbohydrates include pentose sugars of any description and stereoisomerism (e.g., D/L aldopentoses and D/L ketopentoses). C5 carbohydrates include (by way of example and not limitation) arabinose, lyxose, ribose, ribulose, xylose, and xylulose.

“C6 carbohydrate” refers to any carbohydrate, without limitation, that has six (6) carbon atoms. The definition includes hexose sugars of any description and stereoisomerism (e.g., D/L aldohexoses and D/L ketohexoses). C6 carbohydrates include (by way of example and not limitation) allose, altrose, fructose, galactose, glucose, gulose, idose, mannose, psicose, sorbose, tagatose, and talose.

Industrial bio-products can include, but are not limited to monoterpenes, diterpenes, triterpenes, tetraterpenes, sesquiterpene, polyterpene, abietadiene, amorphadiene, carene, α-farnesene, β-farnesene, farnesol, geraniol, geranylgeraniol, linalool, limonene, myrcene, nerolidol, ocimene, patchoulol, β-pinene, sabinene, γ-terpinene, terpinene, valencene. Industrial bio-products can also include, but are not limited to, 2-keto acids, malonyl-CoA, acetoacetyl-CoA and/or ethanol. Industrial bioproducts can further include, but are not limited to, non-fermentative alcohols (e.g., 1-propanol, 1-butanol, isobutanol, 2-methyl-1-butanol, 3-methyl-1-butanol, 3-methyl-1-pentanol, 4-methyl-1-pentanol and 1-hexanol), fatty acid-derived hydrocarbons (fatty alcohols, fatty esters, olefins, and alkanes), and fermentative alcohols (e.g., butanol).

An “obligate anaerobe” is an anaerobe for which atmospheric levels of oxygen can be lethal. Examples of obligate anaerobes include, but are not limited to, Clostridium, Eurobacterium, Bacteroides, Peptostreptococcus, Butyribacterium, Veillonella, and Actinomyces.

A “facultative anaerobe” is an anaerobe that is capable of performing aerobic respiration in the presence of oxygen and is capable of performing anaerobic fermentation under oxygen-limited or oxygen-free conditions. Examples of facultative anaerobes include, but are not limited to, Escherichia, Pantoea, and Streptomyces.

“Synthesis gas” or “syngas” is a gas which includes, but is not limited to, carbon monoxide and hydrogen. Syngas can also include carbon dioxide, water, and/or nitrogen.

“Substantially oxygen-free” conditions can refer to conditions under which anaerobic organisms can grow and/or produce the desired products.

“Isoprene” refers to 2-methyl-1,3-butadiene (CAS#78-79-5). It can refer to the direct and final volatile C5 hydrocarbon product from the elimination of pyrophosphate from 3,3-dimethylallyl pyrophosphate (DMAPP). It may not involve the linking or polymerization of one or more isopentenyl diphosphate (IPP) molecules to one or more DMAPP molecules. Isoprene is not limited by the method of its manufacture.

“Mass yield” is the percentage by mass of a carbon source (e.g., syngas) that is converted to a desired product, such as isoprene, not including water.

The “maximum theoretical mass yield” is the stoichiometrically highest percentage by mass of a carbon source (e.g., syngas) that can be converted to a desired product, such as isoprene, and/or may not include water.

The “experimental mass yield” is the percentage by mass of a carbon source (e.g., syngas) that is converted to a desired product, such as isoprene, and/or may not include water, when such a conversion is carried out. The experimental mass yield is determined by comparing the measured amount of the carbon source introduced to the measured amount of the product produced. The experimental mass yield should be equal to or less than the maximum theoretical mass yield.

“Mevalonate” includes mevalonic acid as well as the anion of mevalonic acid which is the predominant form in biological media. Mevalonic acid is a precursor in the biosynthetic pathway, known as the mevalonate pathway that produces terpenes and steroids. Mevalonate is the primary precursor of isoprenyl pyrophosphate that is in turn the basis for all terpenoids.

“Peak absolute productivity” can refer to the maximum absolute amount of isoprene in the off-gas during the culturing of cells for a particular period of time (e.g., the culturing of cells during a particular fermentation run).

“Peak absolute productivity time point” can refer to the time point during a fermentation run when the absolute amount of isoprene in the off-gas is at a maximum during the culturing of cells for a particular period of time (e.g., the culturing of cells during a particular fermentation run).

“Peak specific productivity” can refer to the maximum amount of isoprene produced per cell during the culturing of cells for a particular period of time (e.g., the culturing of cells during a particular fermentation run). By “peak specific productivity time point” can refer to the time point during the culturing of cells for a particular period of time (e.g., the culturing of cells during a particular fermentation run) when the amount of isoprene produced per cell is at a maximum. The specific productivity can be determined by dividing the total productivity by the amount of cells, as determined by optical density at 600 nm (OD600).

“Cumulative total productivity” can refer to the cumulative, total amount of isoprene produced during the culturing of cells for a particular period of time (e.g., the culturing of cells during a particular fermentation run). In some aspects, the cumulative, total amount of isoprene is measured.

A “nucleic acid” refers to two or more deoxyribonucleotides and/or ribonucleotides in either single or double-stranded form. It is to be understood that mutations, including single nucleotide mutations, can occur within a nucleic acid as defined herein.

A “recombinant nucleic acid” refers to a nucleic acid of interest that is free of one or more nucleic acids (e.g., genes) which, in the genome occurring in nature of the organism from which the nucleic acid of interest is derived, flank the nucleic acid of interest. The term therefore includes, for example, a recombinant DNA which is incorporated into a vector, into an autonomously replicating plasmid or virus, or into the genomic DNA of an anaerobic microorganism, or which exists as a separate molecule (e.g., a cDNA, a genomic DNA fragment, or a cDNA fragment produced by PCR or restriction endonuclease digestion) independent of other sequences. A recombinant nucleic acid may be obtained using molecular biology techniques that are known in the art, or part or all of a recombinant nucleic acid may be chemically synthesized.

A “heterologous nucleic acid” can be a nucleic acid whose nucleic acid sequence is from another species than the host cell or another strain of the same species of the host cell. In some aspects, the sequence is not identical to that of another nucleic acid naturally found in the same host cell. In some aspects, a heterologous nucleic acid is not identical to a wild-type nucleic acid that is found in the same host cell in nature.

An “endogenous nucleic acid” is a nucleic acid whose nucleic acid sequence is naturally found in the host cell. In some aspects, an endogenous nucleic acid is identical to a wild-type nucleic acid that is found in the host cell in nature. In some aspects, one or more copies of endogenous nucleic acids are introduced into a host cell (e.g., anaerobic microorganism).

“Polypeptides” includes polypeptides, proteins, peptides, fragments of polypeptides, fusion polypeptides and variants.

A “heterologous polypeptide” is a polypeptide encoded by a heterologous nucleic acid. In some aspects, the sequence is not identical to that of another polypeptide encoded by a nucleic acid naturally found in the same host cell.

As used herein, the terms “isoprene synthase,” “isoprene synthase variant”, and “IspS,” refer to enzymes that catalyze the elimination of pyrophosphate from dimethylallyl diphosphate (DMAPP) to form isoprene. Isoprene synthase enzymes belong to the enzyme classification group EC 4.2.3.27. An “isoprene synthase” may be a wild type sequence or an isoprene synthase variant.

An “isoprene synthase variant” indicates a non-wild type polypeptide having isoprene synthase activity. One skilled in the art can measure isoprene synthase activity using known methods. See, for example, by GC-MS (see, e.g., WO 2009/132220, Example 3) or Silver et al., J. Biol. Chem. 270:13010-13016, 1995. Variants may have substitutions, additions, deletions, and/or truncations from a wild type isoprene synthase sequence. Variants may have substitutions, additions, deletions, and/or truncations from a non-wild type isoprene synthase sequence. The variants described herein may contain at least one amino acid residue substitution from a parent isoprene synthase polypeptide. In some embodiments, the parent isoprene synthase polypeptide is a wild type sequence. In some embodiments, the parent isoprene synthase polypeptide is a non-wild type sequence. In some embodiments, the parent isoprene synthase polypeptide is a naturally occurring sequence.

As used herein, the term “isopentenyl diphosphate isomerase (IDI)” refers to an enzyme having activity for conversion of isopentenyl pyrophosphate to dimethylallyl pyrophosphate (DMAPP). The enzyme belongs to the classification group EC 5.3.3.2.

As used herein, the term “DXS” refers to the enzyme 1-deoxy-D-xylulose-5-phosphate synthase that converts pyruvate and D-glyceraldehyde 3-phosphate into 1-deoxy-d-xylulose 5-phosphate. This enzyme belongs to the classification group EC 2.2.1.7.

An “endogenous polypeptide” is a polypeptide whose amino acid sequence is naturally found in the host cell. In some aspects, an endogenous polypeptide is identical to a wild-type polypeptide that is found in the host cell in nature.

As used herein, the term “terpenoid” or “isoprenoids” refers to a large and diverse class of naturally-occurring organic chemicals similar to terpenes. Terpenoids are derived from five-carbon isoprene units assembled and modified in a variety of ways, and are classified in groups based on the number of isoprene units used in group members. Hemiterpenoids have one isoprene unit. Monoterpenoids have two isoprene units. Sesquiterpenoids have three isoprene units. Diterpenoids have four isoprene units. Sesterterpenoids have five isoprene units. Triterpenoids have six isoprene units. Tetraterpenoids have eight isoprene units. Polyterpenoids have more than eight isoprene units.

As used herein, “isoprenoid precursor” refers to any molecule that is used by organisms in the biosynthesis of terpenoids or isoprenoids. Non-limiting examples of isoprenoid precursor molecules include, e.g., mevalonate (MVA), isopentenyl pyrophosphate (IPP) and dimethylallyl diphosphate (DMAPP).

As used herein, the terms “phosphoketolase,” “phosphoketolase enzyme,” or “phosphoketolase polypeptide” are used interchangeably and refer to a polypeptide that converts 5-phosphate to glyceraldehyde 3-phosphate and acetyl phosphate and/or converts fructose 6-phosphate to erythrose 4-phosphate and acetyl phosphate. Generally, phosphoketolases act upon ketoses. In certain embodiments, the phosphoketolase polypeptide catalyzes the conversion of xylulose 5-phosphate to glyceraldehyde 3-phosphate and acetyl phosphate. In other embodiments, the phosphoketolase polypeptide catalyzes the conversion of fructose 6-phosphate to erythrose 4-phosphate and acetyl phosphate. In other embodiments, the phosphoketolase polypeptide catalyzes the conversion of sedoheptulose-7-phosphate to a product (e.g., ribose-5-phosphate) and acetyl phosphate.

It is understood that any combinations of upper and lower ranges of the carbon and/or energy sources disclosed herein are contemplated within the scope of the invention.

As used herein and in the appended claims, the singular forms “a,” “or,” and “the” include plural referents unless the context clearly dictates otherwise.

Reference to “about” a value or parameter herein includes (and describes) embodiments that are directed to that value or parameter per se. For example, description referring to “about X” includes a description of “X.”

It is understood that aspects and variations of the methods, uses, compositions, formulations, articles of manufacture, kits, medicaments, or unit dosage forms described herein include “consisting of” and/or “consisting essentially of” aspects and variations.

### Anaerobic Pathways

The invention provides for compositions of anaerobic organisms capable of making isoprene (and other products) using syngas, methods of making and using such organisms for producing isoprene and other products under substantially oxygen-free conditions. Accordingly, in one aspect, the invention features compositions and methods for the production of isoprene by anaerobic organisms.

The mechanism for conversion of syngas to isoprene can be as follows. CO and CO₂ are converted to acetylCoA via the Wood-Ljungdahl pathway, as shown in Equation 1, wherein “X(red)” represents an electron donor in its reduced form.

CO+CO₂+3X(red)+ATP→AcetylCoA Equation 1

AcetylCoA is converted to isoprene via the MVA pathway as shown in Equation 2, wherein “XH₂” is the hydrogenated form of electron donor X.

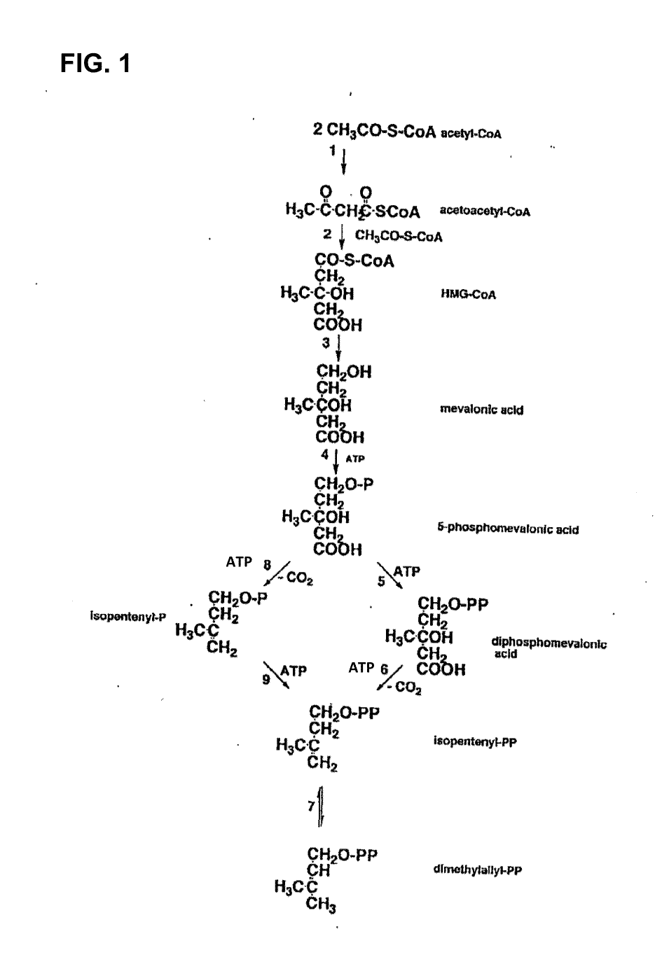

The classical and modified MVA pathways are illustrated in FIG. 1, including the enzymes acetyl-CoA acetyltransferase (AACT); 2, HMG-CoA synthase (HMGS); 3, HMG-CoA reductase (HMGR); 4, mevalonate kinase (MVK); 5, phosphomevalonate kinase (PMK); 6, diphosphomevalonate decarboxylase (MVD or DPMDC); 7, isopentenyl diphosphate isomerase (IDI); 8, phosphomevalonate decarboxylase (PMDC); 9, isopentenyl phosphate kinase (IPK). The classical MVA pathway proceeds from reaction 1 through reaction 7 via reactions 5 and 6, while a modified MVA pathway goes through reactions 8 and 9. P and PP in the structural formula are phosphate and pyrophosphate, respectively. This figure was taken from Koga and Morii, Microbiology and Mol. Biology Reviews, 71:97-120, 2007, which is incorporated by reference in its entirety, particularly with respect to nucleic acids and polypeptides of the modified MVA pathway. The modified MVA pathway is present, for example, in some archaeal organisms, such as Methanosarcina mazei.

3 AcetylCoA+3 ATP+2XH₂C₅H₈+CO₂ Equation 2

Water and CO are converted to CO2 as shown in Equation 3.

H₂O+CO→CO₂+X(red) Equation 3

The oxidized form of the electron donor is reduced by a hydrogenase as shown in Equation 4.

H₂+X(ox)→2H⁺+X(red) Equation 4

Ferredixon is oxidized as shown in Equation 5, wherein “Fd (red)” represents reduced ferredoxin, and “Fd (ox)” represents oxidized ferredoxin.

Fd(red)+NAD+→NADH+Fd(ox)+2H+ Equation 5

The protons produced via Equation 5 are consumed in the production of ATP via oxidative phosphorylation, as shown in Equation 6.

2-3H+→ATP Equation 6

The yield of isoprene from syngas depends upon the composition of the syngas. The generalized stoichiometric equation for the conversion of syngas to isoprene is shown in Equation 7.

(5-n)CO₂+nCO+(14-n)H₂+(n-10)H₂O→1Isoprene+(n-5)CO₂+(10-n)H₂O Equation 7

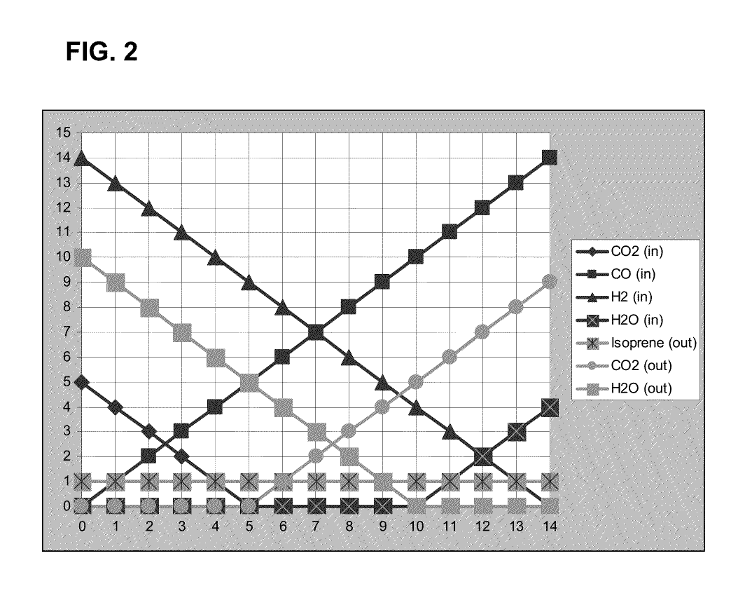

The moles of CO₂, CO, H₂, and H₂O in the syngas and the resulting stoichiometric molar yields of isoprene, CO₂, and H₂O according to Equation 7 are shown in Table 1 for n of 0 through 14.
The same values are depicted graphically in FIG. 2. Negative values generated from the equation can be treated as zero. Values of zero indicate that a component of the equation is absent from the syngas or is not produced as a product of the reaction. For example, in some aspects the syngas lacks one or more of CO₂, CO, H₂, and H₂O. In some aspects, CO₂ or H₂O are not produced by the reaction. Any combinations of the synthesis gases disclosed herein are contemplated within the scope of the invention.

TABLE 1

| Moles | Moles | Moles | Moles | | Moles | Moles | Moles |
|---|---|---|---|---|---|---|---|
| of CO₂ | of CO | of H₂ | of H₂O | | of C₅H₈ | of CO₂ | of H₂O |
| react- | react- | react- | react- | | pro- | pro- | pro- |
| ing | ing | ing | ing | → | duced | duced | duced |
| 0 | 14 | 0 | 4 | | 1 | 9 | 0 |
| 0 | 13 | 1 | 3 | | 1 | 8 | 0 |
| 0 | 12 | 2 | 2 | | 1 | 7 | 0 |
| 0 | 11 | 3 | 1 | | 1 | 6 | 0 |
| 0 | 10 | 4 | 0 | | 1 | 5 | 0 |
| 0 | 9 | 5 | 0 | | 1 | 4 | 1 |
| 0 | 8 | 6 | 0 | | 1 | 3 | 2 |
| 0 | 7 | 7 | 0 | | 1 | 2 | 3 |
| 0 | 6 | 8 | 0 | | 1 | 1 | 4 |
| 0 | 5 | 9 | 0 | | 1 | 0 | 5 |
| 1 | 4 | 10 | 0 | | 1 | 0 | 6 |
| 2 | 3 | 11 | 0 | | 1 | 0 | 7 |
| 3 | 2 | 12 | 0 | | 1 | 0 | 8 |
| 4 | 1 | 13 | 0 | | 1 | 0 | 9 |
| 5 | 0 | 14 | 0 | | 1 | 0 | 10 |

The theoretical mass yield of isoprene, not including water, can be calculated for a given composition of syngas. This is done by dividing the mass of isoprene produced by the mass of all products produced except water and multiplying by 100 to get a percentage. For example, for synthesis gas comprising 8 moles CO, 6 moles H₂, and 0 moles each of CO₂ and H₂O, the stoichiometric products are 1 mole isoprene, 3 moles CO₂, and 2 moles H₂O. The total mass of isoprene produced is 1 mole×68 grams/mole (the molar mass of isoprene)=68 grams. The total mass of CO₂ produced is 3 moles×44 grams/mole (the molar mass of CO₂)=132 grams. The total mass of all products of the reaction, excluding water, is 68 grams+132 grams=200 grams. The theoretical mass yield of isoprene is 68 grams/200 grams*100=34%.

The composition of syngas can vary depending upon the feedstock from which the syngas is derived. Specifically the ratios of C, H, and O in the feedstock will determine the ratio of carbon monoxide to hydrogen in the resulting syngas. Table 2 shows the C, H, and O compositions and the resulting optimal carbon monoxide and hydrogen compositions of syngas derived from the following feedstocks: carbohydrates, biomass, coal, rubber tires, and municipal solid waste. The syngas compositions are provided as “optimal” syngas compositions because the high-temperature syngas reactor might use some oxygen and therefore lose some carbon to carbon monoxide, hydrogen, water, biomass conversion to carbon or unconverted biomass. Additionally, the C, H, and O compositions of the feedstocks may vary somewhat from those given in Table 1. Syngas may also produced from gasification of a mixture (e.g., blend) of carbohydrates, biomass, coal, rubber tires, municipal solid waste, or a mixture thereof, e.g., gasification of a mixture of coal and biomass.

Industrial waste gases may be used in producing isoprene with no or only minimal additional scrubbing or pre-treatment steps being used to make the gases suitable therefor. The waste gases may result from any number of industrial processes. The invention has particular applicability to supporting the production of isoprene from gaseous substrates such as high volume CO-containing industrial flue gases. Examples include gases produced during ferrous metal products manufacturing, non-ferrous products manufacturing, petroleum refining processes, gasification of coal, gasification of biomass, electric power production, carbon black production, ammonia production, methanol production and coke manufacturing. In a particular embodiment of the invention, the waste gases are generated during a process for making steel. For example, those skilled in the art will appreciate the waste gases produced during various stages of the steel making process have high CO and/or CO₂ concentrations. In particular, the waste gas produced during the decarburisation of steel in various methods of steel manufacturing, such as in an oxygen converter (e.g. BOF or KOBM), has a high CO content and low O₂ content making it a suitable substrate for any of the methods of producing isoprene described herein. Methods relating to using gases and biomass materials are disclosed in U.S. Publication Nos. 2010/0323417 and 2010/0317074.

The maximum theoretical mass yield of isoprene can be determined, as described above, for a given composition of syngas. Since the syngas composition can be determined for a given feedstock, the theoretical maximum isoprene mass yield can be determined for a given feedstock. The theoretical maximum isoprene mass yields for sugar, biomass, coal, rubber tires, and municipal solid waste feedstocks are given in Table 2. The maximum theoretical mass yield of isoprene from syngas derived from carbohydrates (e.g. sugar) can be about 32%, in some aspects about 32.4%, the same as for conversion of carbohydrates to isoprene by aerobic organisms in the presence of oxygen. Higher maximum theoretical mass yields of isoprene can be obtained using syngas derived from other feedstocks, such as biomass, coal, rubber tires, and municipal solid waste.

TABLE 2

| | | | Maximum |
|---|---|---|---|
| | | Optimal | Theoretical Isoprene |
| | | Syngas CO | Mass Yield |
| Feedstock | C, H, and O | and H₂ | (not including |
| Compositions | Compositions | Compositions | water) |
| Sugar | C—H₂—O + 0 H₂O | CO + 1 H₂ | 32.4% |
| Biomass | C—H_{1.4}—O_{0.6} + | CO + 1.1 H₂ | 44.4% |
| | 0.4 H₂O | | |
| Coal | C—H + H₂O | CO + 1.5 H₂ | 93.6% |
| Rubber Tires | C—H_{1.6} + H₂O | CO + 1.8 H₂ | 100.2% |
| Municipal Solid | C—H_{2.3}—O_{0.6} + | CO + 1.55 H₂ | 51.9% |
| Waste | 0.4 H₂O | | |

The composition of syngas can also vary depending upon the method by which feedstock is converted to syngas. For example, syngas produced by water reforming reactions, oxygen reforming reactions, and oxygen and water reforming reactions can have difference compositions for the same feedstock. Accordingly, the maximum theoretical mass yield of isoprene from syngas derived from a given feedstock can vary depending on the method by which the syngas is produced. Exemplary compositions of syngas produced from sugar, biomass, coal, rubber tires, and municipal solid waste feedstocks by water reforming reactions are given in Table 3. Also provided in Table 3 is the exemplary maximum theoretical mass yield of isoprene for each of these syngas compositions.

TABLE 3

| | | | Maximum |
|---|---|---|---|
| Feedstock | C, H, and O | CO and H₂ | Isoprene |
| Compositions | Compositions | Compositions | Yield |
| Sugar | C—H₂—O + 0 H₂O | CO + 1 H₂ | 32.4% |
| Biomass | C—H_{1.4}—O_{0.6} + | CO + 1.1 H₂ | 44.4% |
| | 0.4 H₂O | | |
| Coal | C—H + H₂O | CO + 1.5 H₂ | 93.6% |
| Rubber Tires | C—H_{1.6} + H₂O | CO + 1.8 H₂ | 100.2% |
| Municipal Solid | C—H_{2.3}—O_{0.6} + | CO + 1.55 H₂ | 51.9% |
| Waste | 0.4 H₂O | | |

The exemplary compositions of syngas produced from sugar, biomass, coal, rubber tires, and municipal solid waste feedstocks by oxygen reforming reactions are given in Table 4. Also provided in Table 4 is the exemplary maximum theoretical mass yield of isoprene for each of these syngas compositions.

TABLE 4

| | | | Maximum |
|---|---|---|---|
| Feedstock | C, H, and O | CO and H₂ | Isoprene |
| Compositions | Compositions | Compositions | Yield |
| Sugar | C—H₂—O + 0.1 O₂ | CO + 0.8 H₂ | 29.2% |
| Biomass | C—H_{1.4}—O_{0.6} + 0.2 O₂ | CO + 0.7 H₂ | 36.0% |
| Coal | C—H + 0.5 O₂ | CO + 0.5 H₂ | 56.1% |
| Rubber Tires | C—H_{1.6} + 0.5 O₂ | CO + 0.8 H₂ | 64.4% |
| Municipal Solid | C—H_{2.3}—O_{0.6} + 0.2 O₂ | CO + 1.15 H₂ | 43.8% |
| Waste | | | |

The exemplary compositions of syngas produced from sugar, biomass, coal, rubber tires, and municipal solid waste feedstocks by oxygen and water reforming reactions are given in Table 5. Also provided in Table 5 is the exemplary maximum theoretical mass yield of isoprene for each of these syngas compositions.

TABLE 5

| | | | Maximum |
|---|---|---|---|
| Feedstock | | CO and H₂ | Isoprene |
| Compositions | C, H, and O Compositions | Compositions | Yield |
| Sugar | C—H₂—O + 0 H₂O + 0.1 O₂ | CO + 0.8 H₂ | 29.2% |
| Biomass | C—H_{1.4}—O_{0.6} + 0.2 H₂O + | CO + 0.9 H₂ | 40.2% |
| | 0.1 O₂ | | |
| Coal | C—-H + 0.5 H₂O + 0.25 O₂ | CO + 1 H₂ | 74.9% |
| Rubber Tires | C—H_{1.6} + 0.5 H₂O + 0.25 O₂ | CO + 1.3 H₂ | 82.3% |
| Municipal | C—H_{2.3}—O_{0.6} + 0.2 H₂O + | CO + 1.35 H₂ | 47.8% |
| Solid | 0.1 O₂ | | |
| Waste | | | |

In some aspects, the cells of any of the compositions or methods described herein produce the maximum theoretical mass yield of isoprene for the particular syngas composition used as a carbon source. In some aspects, the cells produce at least about 5, 10, 20, 30, 40, 50, 60, 70, 80, 90, or 95% of the maximum theoretical mass yield of isoprene.

In contrast, in one aspect, the maximum theoretical mass yield for isoprene production from glucose in the presence of oxygen can be about 32%, in some aspects about 32.4%. For E. coli engineered to comprise isoprene synthase and an MVA pathway, the experimentally obtained mass yield of isoprene from glucose via the MVA pathway can be 25.2%, which is about 78% of the maximum theoretical mass yield. For E. coli engineered to include isoprene synthase, an MVA pathway, and one or more copies of a heterologous DXP pathway or one or more additional copies of an endogenous DXP, the experimentally obtained mass yield of isoprene from glucose can be about 30%, in some aspects about 30.2%, which is about 93% of the maximum theoretical mass yield.

Production of isoprene from syngas by anaerobic organisms provides a number of improvements over production of isoprene from sugars by aerobic organisms. First, the maximum theoretical mass yield of isoprene can be greater for the anaerobic organisms, as discussed further below. Second, the anaerobic organisms do not have excess reducing power in the form of NAD(P)H that must be turned over via cell growth, formation of byproducts (such as glycerol, lactic acid, or ethanol) or oxidation using molecular oxygen. Without this NAD(P)H turnover requirement, anaerobic organisms can have higher energy yield, lower oxygen demand, lower heat of fermentation, and lower utility costs to run the process. Third, due to the lack of oxygen in the system, anaerobic organisms can have greater isoprene concentration in the offgas, lower probability of creating a flammable isoprene-oxygen mixture, easier recovery, and higher isoprene quality. Fourth, the anaerobic organisms can be more easily grown by using existing infrastructure, such as existing plants designed for production of bioethanol.

Accordingly, in some aspects, the anaerobic cells of any of the compositions or methods described herein are capable of producing of isoprene with a maximum theoretical mass yield of at least about 32%, for example, at least about 32.4%. In some aspects, the maximum theoretical mass yield is at least about 32% when the carbon source is a sugar of formula (CH₂O)ₙ where n is typically 5 or 6. In some aspects, maximum theoretical mass yield is greater than about 32%, for example, at least about 32.4%. In some aspects, maximum theoretical mass yield is greater than about 40, 50, 60, 70, 80, or 90%. In some aspects, the maximum theoretical mass yield is about 100%. In some aspects, the cells of any of the compositions or methods described herein are capable of producing of isoprene with an experimental mass yield that is greater than about 78% of the maximum theoretical mass yield. In some aspects, the experimental mass yield is greater than about 80, 85, 90, 95, 96, 97, 98, or 99% of the maximum theoretical mass yield. In some aspects, the experimental mass yield is about 100% of the maximum theoretical mass yield.

In some aspects, the anaerobic cells of any of the compositions or methods described herein are capable of producing isoprene wherein the amount of any single byproduct produced by the cells (e.g., glycerol, lactic acid, or ethanol) is less than the amount of isoprene produced. In some aspects, the amount of any single byproduct is less than about 90, 80, 70, 60, 50, 40, 30, 20, 10, 5, 2, or 1% of the amount of isoprene produced. In some aspects, the total amount of byproducts (e.g., glycerol, lactic acid, or ethanol) is less than the amount of isoprene produced. In some aspects, the total amount of byproducts is less than about 90, 80, 70, 60, 50, 40, 30, 20, 10, 5, 2, or 1% of the amount of isoprene produced.

### Obligate Anaerobes

There are numerous types of anaerobic cells that can be used in the compositions and methods of the present invention. In one aspect of the invention, the cells described in any of the compositions or methods described herein are obligate anaerobic cells. Obligate anaerobes typically do not grow well, if at all, in conditions where oxygen is present. Growth conditions are discussed in greater detail below. It is to be understood that a small amount of oxygen may be present, that is, there is some tolerance level that obligate anaerobes have for a low level of oxygen. In one aspect, obligate anaerobes engineered to produce isoprene are grown under substantially oxygen-free conditions wherein the amount of oxygen present is not harmful to the growth, maintenance, and/or fermentation of the anaerobes. In another aspect, obligate anaerobes engineered to produce other desired products such as industrial enzymes are grown under substantially oxygen-free conditions wherein the amount of oxygen present is not harmful to the growth, maintenance, and/or fermentation of the anaerobes.

Provided herein are obligate anaerobic cells comprising one or more heterologous nucleic acids encoding an isoprene synthase polypeptide, wherein the cells are capable of producing isoprene in a substantially oxygen-free culture condition. In some embodiments, a carbohydrate is used as energy and/or carbon source. In some embodiments, a carbohydrate and hydrogen are used as energy and/or carbon source. In some embodiments, synthesis gas is used as energy and/or carbon source. In some aspects, the isoprene synthase polypeptide is less susceptible to degradation (e.g., degradation by protease(s)) in the cells during culturing.

In some aspects, the isoprene synthase polypeptide is less susceptible to degradation in the cells when using inducible promoter or constitutive promoter (e.g., low expression constitutive promoter) for driving the expression of isoprene synthase polypeptide. In some aspects, the degradation of isoprene synthase polypeptide in the cells when using the inducible promoter or constitutive promoter (e.g., low expression constitutive promoter) is less compared to the degradation when using a constitutive promoter and/or high expression promoter (e.g., high expression constitutive promoter) for driving expression of the isoprene synthase polypeptide.

In some aspects, the isoprene synthase polypeptide is less susceptible to degradation in the cells when using the host anaerobic cells (e.g., cells that are deficient in protease(s)) in which the isoprene synthase polypeptide is not degraded or more resistant to degradation by protease(s). In some aspects, the degradation of isoprene synthase polypeptide in the cells when using such host anaerobic cells is less compared to the degradation of isoprene synthase polypeptide in the cells when not using such host anaerobic cells.

In some aspects, the isoprene synthase polypeptide is less susceptible to degradation in the cells when using isoprene synthase polypeptide (e.g., a variant) having more resistance to degradation by protease(s) in the cells. In some aspects, the isoprene synthase polypeptide (e.g., a variant) has mutation(s) in the wild-type or naturally occurring isoprene synthase, and wherein the isoprene synthase polypeptide having mutation(s) is more resistant to degradation by protease(s). In some aspects, the degradation of isoprene synthase polypeptide in the cells when using such isoprene synthase polypeptide is less compared to the degradation of isoprene synthase polypeptide in the cells when not using such isoprene synthase polypeptide. In some aspects, the degradation of isoprene synthase polypeptide in the cells when using such isoprene synthase polypeptide is less compared to the degradation of isoprene synthase polypeptide in the cells when using a wild-type or naturally occurring isoprene synthase.

In some aspects, the isoprene synthase polypeptide is less susceptible to degradation in the cells when using (a) inducible promoter or constitutive promoter (e.g., low expression constitutive promoter) for driving the expression of isoprene synthase polypeptide, (b) using the host anaerobic cells (e.g., cells that are deficient in protease(s)) in which the isoprene synthase polypeptide is not degraded or more resistant to degradation by protease(s), and/or (c) using isoprene synthase polypeptide (e.g., a variant) having more resistance to degradation by protease(s) in the cells. In some aspects, the degradation when using (a), (b), and/or (c) is less compared to the degradation when not using (a), (b), and/or (c).

In some aspects, the obligate anaerobic cells are bacteria cells. In some aspects, the obligate anaerobic cells are any of Clostridium, Eurobacterium, Bacteroides, Peptostreptococcus, Butyribacterium, Veillonella, and Actinom. The obligate anaerobic cells described herein may be Clostridium ljungdahlii, Clostridium aceticum, Clostridium acetobutylicum, Moorella thermoacetica, Clostridium autoethanogenum, Eurobacterium limosum, Clostridium carboxydivorans, Peptostreptococcus productus, Rhodospirillum rubrum, Desulfitobacterium hafniense, Aecetoanaerobium notera, Butyribacterium methylotrophicum, Thermoanaerobacter kivui, Eubacterium limosum, Peptostreptococcus productus, or Acetobacterium woodi.

In some aspects, the obligate anaerobic cells are mesophilic. Examples of mesophilic anaerobes that may be used in the present invention are Clostridium autoethanogenum, Clostridium ljungdahlii, Clostridium carboxydivorans, Oxobacter pfennigii, Peptostreptococcus productus, Acetobacterium woodii, Eurobacterium limosum, Butyribacterium methylotrophicum, Rubivivax gelatinosus, Rhodopseudomonas palustris P4, Rhodospirillum rubrum, Citrobacter sp Y19, Methanosarcina barkeri, and Methanosarcina acetovorans strain C2A. In some aspects, the obligate anaerobic cells are thermophilic. Examples of thermophilic anaerobes that may be used in the present invention are Moorella thermoacetica, Moorella thermoautotrophica, Moorella strain AMP, Carboxydothermus hydrogenoformans, Carboxydibrachium pacificus, Carboxydocella sporoproducens, Carboxydocella thermoautotrophica, Thermincola carboxydiphila, Thermincola ferriacetica, Thermolithobacter carboxydivorans, Thermosinus carboxydivorans, Desulfotomaculum kuznetsovii, Desulfotomaculum thermobenzoicum subspecies thermosyntrophicum, Desulfotomaculum carboxydivorans, Methanothermobacter thermoautotrophicus, Thermococcus strain AM4, and Archaeoglobus fulgidus. In some aspects, the obligate anaerobe is selected from the group consisting of Clostridium ljungdahlii, Clostridium autoethanogenum, Eurobacterium limosum, Clostridium carboxydivorans, Peptostreptococcus productus, and Butyribacterium methylotrophicum. In some aspects, the obligate anaerobic cells are Clostridium cells. The obligate anaerobic cells may be Clostridium ljungdahlii, Clostridium aceticum, Clostridium acetobutylicum, Clostridium carboxidivorans, or Clostridium autoethanogenum. In some aspects, the obligate anaerobic cells are acetobacterium cells. In some aspects, the obligate anaerobic cells are Acetobacterium woodii.

In some aspects, the obligate anaerobic cells are acetogen cells. Over 100 acetogenic species are known from a variety of habitats. The group of the acetogens involves 22 different genera, in which Clostridium and Acetobacterium are the best known acetogenic species, as described in Drake, H. L. et al., 2008. Ann. NY Acad. Sci. 1125: 100-128, the contents of which are expressly incorporated herein by reference in its entirety with respect to acetogenic species. In some aspects, the cells are Clostridium (e.g., Clostridium ljungdahlii, Clostridium aceticum, Clostridium carboxidivorans, Clostridium autoethanogenum). In some aspects, the cells are Acetobacterium (e.g., Acetobacterium woodii). In some aspects, the cells are any of Clostridium ljungdahlii, Clostridium aceticum, Moorella thermoacetica (also known as Clostridium thermoaceticum), Rhodospirillum rubrum, Desulfitobacterium hafniense, Clostridium carboxidivorans, Aecetoanaerobium notera, Butyribacterium methylotrophicum, Thermoanaerobacter kivui, Eubacterium limosum, Peptostreptococcus productus, Acetobacterium woodi, Desulfococcus oleovorans, Syntrophobacter fumaroxidans, delta proteobacterium MLMS-1, Treponema primitia ZAS-1, Treponema primitia ZAS-2, Carboxydothermus hydrogenoformans, Sporomsa termitida, Clostridium difficile, or Alkaliphilus metalliredigens.

### Facultative Anaerobes

In another aspect of the invention, the cells described and/or used in any of the compositions or methods described herein are facultative anaerobic cells. Thus, provided herein are facultative anaerobic cells comprising one or more heterologous nucleic acids encoding an isoprene synthase polypeptide, wherein the cells are capable of producing isoprene in a substantially oxygen-free culture condition. In some embodiments, a carbohydrate is used as energy and/or carbon source. In some embodiments, a carbohydrate and hydrogen are used as energy and/or carbon source. In some embodiments, synthesis gas is used as energy and/or carbon source. In some aspects, the facultative anaerobic cells are bacteria cells. In some aspects, the isoprene synthase polypeptide is less susceptible to degradation (e.g., degradation by protease(s)) in the cells during culturing.

In some aspects, the isoprene synthase polypeptide is less susceptible to degradation in the cells when using inducible promoter or constitutive promoter (e.g., low expression constitutive promoter) for driving the expression of isoprene synthase polypeptide. In some aspects, the degradation of isoprene synthase polypeptide in the cells when using the inducible promoter or constitutive promoter (e.g., low expression constitutive promoter) is less compared to the degradation when using a constitutive promoter and/or high expression promoter (e.g., high expression constitutive promoter) for driving expression of the isoprene synthase polypeptide.

In some aspects, the isoprene synthase polypeptide is less susceptible to degradation in the cells when using the host anaerobic cells (e.g., cells that are deficient in protease(s)) in which the isoprene synthase polypeptide is not degraded or more resistant to degradation by protease(s). In some aspects, the degradation of isoprene synthase polypeptide in the cells when using such host anaerobic cells is less compared to the degradation of isoprene synthase polypeptide in the cells when not using such host anaerobic cells.

In some aspects, the isoprene synthase polypeptide is less susceptible to degradation in the cells when using isoprene synthase polypeptide (e.g., a variant) having more resistance to degradation by protease(s) in the cells. In some aspects, the isoprene synthase polypeptide (e.g., a variant) has mutation(s) in the wild-type or naturally occurring isoprene synthase, and wherein the isoprene synthase polypeptide having mutation(s) is more resistant to degradation by protease(s). In some aspects, the degradation of isoprene synthase polypeptide in the cells when using such isoprene synthase polypeptide is less compared to the degradation of isoprene synthase polypeptide in the cells when not using such isoprene synthase polypeptide. In some aspects, the degradation of isoprene synthase polypeptide in the cells when using such isoprene synthase polypeptide is less compared to the degradation of isoprene synthase polypeptide in the cells when using a wild-type or naturally occurring isoprene synthase.

In some aspects, the isoprene synthase polypeptide is less susceptible to degradation in the cells when using (a) inducible promoter or constitutive promoter (e.g., low expression constitutive promoter) for driving the expression of isoprene synthase polypeptide, (b) using the host anaerobic cells (e.g., cells that are deficient in protease(s)) in which the isoprene synthase polypeptide is not degraded or more resistant to degradation by protease(s), and/or (c) using isoprene synthase polypeptide (e.g., a variant) having more resistance to degradation by protease(s) in the cells. In some aspects, the degradation when using (a), (b), and/or (c) is less compared to the degradation when not using (a), (b), and/or (c).

In some aspects, the cells are gram-positive bacterial cells. In some aspects, the cells are gram negative bacterial cells. In some aspects, the cells are Streptomyces cells, Escherichia cells or Pantoea cells. In some aspects, the cells are Bacillus subtilis, Streptomyces griseus, Escherichia coli, or Pantoea citrea. In some aspects, the cells are not Escherichia coli.

The facultative anaerobe may be engineered to produce isoprene and/or other products using synthesis gas as its energy source. In some aspects, substantially oxygen-free conditions are used in the fermentation system for the facultative anaerobes.

Expression and stability for proteins (such as isoprene synthase) may also be increased as follows. For example, in some aspects, heterologous gene(s) (e.g., isoprene synthase) are engineered and/or modified for improved expression, and for improved stability of the expressed heterologous protein. Improved expression and improved protein stability may be achieved by controlling expression using a constitutive or inducible promoter. Improved expression and improved protein stability may be achieved by engineering the heterologous gene and/or its promoter so that the stability of the resulting mRNA transcript is increased. Improved expression and improved protein stability may be achieved by engineering the ribosome binding site of the promoter such that translation of the mRNA is improved. Improved expression and improved protein stability may be achieved by codon optimization of one of more heterologous genes. Improved expression and improved protein stability may be achieved by engineering beneficial mutations into the coding sequence of the heterologous gene. Inducible promoter may be any suitable inducible promoter that may be used in the present disclosure or any one of the inducible promoters described herein. For example, an anhydrotetracycline-inducible promoter and/or gene expression system may be used (see Dong et al., Metabolic Engineering 2012 January; 14(1): 59-67).

In some aspects, the host cell is engineered to permit improved expression and improved stability of the expressed heterologous protein(s) (e.g., isoprene synthase). The expression of endogenous or heterologous genes within the host cell, that have previously been demonstrated or implicated in improving protein expression and stability, may be increased or repressed (see Kolaj et al., Microbial Cell Factories 2009, 8:9; Wong, Sui-Lam, Current Opinion in Biotechnology 1995, 6:517-522). These genes include, but are not limited to, chaperones and chaperonins of the following families: proteases (e.g. lonA, lonB), heat shock protein 70 family (e.g. DnaK, DnaJ, GrpE), heat shock protein 60 family (e.g. groEL), heat shock protein 10 family (e.g. groES), small heat shock protein family (e.g. IbpA, IbpB), trigger factor, and miscellaneous accessory molecules (e.g. thioredoxin, ClpB).

### Making Anaerobes Capable of Isoprene Production

Isoprene synthase expressed in anaerobic cells may be susceptible to degradation or cleavage by protease(s) in the anaerobic cells. Proteolysis of isoprene synthase may significantly decrease isoprene production levels, thus the present invention provides strain(s) where isoprene synthase, when introduced to the strain, is not susceptible to degradation. Gene(s) in anaerobic cells coding for the protease(s) that degrade isoprene synthase may be identified and the expression of such gene(s) are disrupted. The strain(s) that do not cause degradation of polypeptide(s) provided herein including IspS may be used for expressing isoprene synthase(s), polypeptide(s) in MVA upper pathway (e.g., polypeptides encoded by mvaE and/or mvaS), polypeptide(s) in MVA lower pathways (MVK, PMK, and/or MVD), IDI and/or DXS.

In some aspects, the isoprene synthase polypeptide is less susceptible to degradation in the cells when using the host anaerobic cells (e.g., cells that are deficient in protease(s)) in which the isoprene synthase polypeptide is not degraded or more resistant to degradation by protease(s). In some aspects, the degradation of isoprene synthase polypeptide in the cells when using such host anaerobic cells is less compared to the degradation of isoprene synthase polypeptide in the cells when not using such host anaerobic cells. In some aspects, the degradation of isoprene synthase polypeptide in the cells when using such host anaerobic cells is less compared to the degradation of isoprene synthase polypeptide in the cells when using wild-type or naturally occurring anaerobic cells. In some aspects, the degradation of isoprene synthase polypeptide in the cells when the host anaerobic cells that are deficient in protease(s) is less compared to the degradation of isoprene synthase polypeptide in the cells when using anaerobic cells that are deficient in protease(s).

Thus, in some aspects of any of the cells or methods provided herein, the cells are deficient in protease(s) (e.g., protease(s) that cleave isoprene synthase). In some aspects, the cells are deficient in protease(s) such that the isoprene synthase polypeptide expressed in the cells is not degraded or more resistant to degradation compared to cells that are not deficient in the protease. Any of the strains where isoprene synthase is not degraded or the degradation of isoprene synthase is reduced compared to naturally occurring strains may be used for expressing any of the polypeptides described herein including heterologous isoprene synthase.

Also provided herein are anaerobic microorganisms that can be engineered to produce isoprene and/or other product(s). In some aspects, the anaerobic microorganism is engineered to produce isoprene. In some aspects, the anaerobic microorganism is engineered to produce product(s) other than isoprene. In certain embodiments, the other products are a natural consequence of isoprene production. In other embodiments, the anaerobe is engineered with additional material (e.g., heterologous nucleic acid encoding desired product(s) or additional copies of endogenous nucleic acid encoding desired product(s)) to produce the product(s) with the isoprene.

In some aspects, the cells are capable of producing one or more products other than isoprene that are selected from the group consisting of: an industrial enzyme, a neutraceutical, a surfactant, an anti-microbial, a biopolymer, an organic acid, a bioplastic monomer, a fermentative alcohol, a non-fermentative alcohol, a fatty alcohol, a fatty acid ester, an isoprenoid alcohol, an alkene, an alkane, and an isoprenoid.

Some types of anaerobes are engineered to produce isoprene as well as one or more products other than isoprene, such as industrial enzyme(s) or other industrial bio-products. Other types of anaerobes are engineered to produce only the industrial enzyme or industrial bio-product, without the isoprene. Industrial enzymes can include, but are not limited to, hemicellulases, cellulases, peroxidases, proteases, metalloproteases, xylanases, lipases, phospholipases, esterases, perhydrolasess, cutinases, pectinases, pectate lyases, mannanases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. Industrial bio-products can include, but are not limited to monoterpenes, diterpenes, triterpenes, tetraterpenes, sesquiterpene, and polyterpene. Industrial bio-products include, but are not limited to, abietadiene, amorphadiene, carene, α-farnesene, β-farnesene, farnesol, geraniol, geranylgeraniol, linalool, limonene, myrcene, nerolidol, ocimene, patchoulol, β-pinene, sabinene, γ-terpinene, terpinene, valencene. Industrial bio-products can also include, but are not limited to, 2-keto acids, malonyl-CoA, acetoacetyl-CoA and/or ethanol. Industrial bioproducts can further include, but are not limited to, non-fermentative alcohols (e.g., 1-propanol, 1-butanol, isobutanol, 2-methyl-1-butanol, 3-methyl-1-butanol, 3-methyl-1-pentanol, 4-methyl-1-pentanol and 1-hexanol), fatty acid-derived hydrocarbons (fatty alcohols, fatty esters, olefins, and alkanes), and fermentative alcohols (e.g., butanol).

### Isoprene Synthase

In some aspects of any one of the compositions (e.g., cells) and methods described herein, the cells comprise at least one nucleic acid encoding an isoprene synthase polypeptide or a polypeptide having isoprene synthase activity. In some aspects, the cells comprise at least one heterologous nucleic acid encoding an isoprene synthase polypeptide or a polypeptide having isoprene synthase activity. In some aspects, the cells comprise additional copy or copies of endogenous nucleic acid encoding an isoprene synthase polypeptide or a polypeptide having isoprene synthase activity. The nucleic acid(s) encoding isoprene synthase polypeptide may be integrated into a genome of the cells. The nucleic acid(s) encoding isoprene synthase polypeptide may be stably expressed in the cells. The nucleic acid(s) encoding isoprene synthase polypeptide may be on a vector. In some aspects, the nucleic acid encoding an isoprene synthase polypeptide is operably linked to a constitutive promoter. In some aspects, the nucleic acid encoding an isoprene synthase polypeptide is operably linked to an inducible promoter. In some aspects, the nucleic acid encoding an isoprene synthase polypeptide is operably linked to a strong promoter. In a particular aspect, the cells are engineered to over-express the isoprene synthase pathway polypeptide relative to wild-type cells. In some aspects, the nucleic acid encoding an isoprene synthase polypeptide is operably linked to a weak promoter. In some aspects, the isoprene synthase polypeptide is a polypeptide from Pueraria or Populus or a hybrid such as Populus alba×Populus tremula.

The nucleic acids encoding an isoprene synthase polypeptide(s) can be integrated into a genome of the host cells or can be stably expressed in the cells. The nucleic acids encoding an isoprene synthase polypeptide(s) can additionally be on a vector.

Exemplary isoprene synthase nucleic acids include nucleic acids that encode a polypeptide, fragment of a polypeptide, peptide, or fusion polypeptide that has at least one activity of an isoprene synthase polypeptide. Isoprene synthase polypeptides convert dimethylallyl diphosphate (DMAPP) into isoprene. Exemplary isoprene synthase polypeptides include polypeptides, fragments of polypeptides, peptides, and fusions polypeptides that have at least one activity of an isoprene synthase polypeptide. Exemplary isoprene synthase polypeptides and nucleic acids include naturally-occurring polypeptides and nucleic acids from any of the source organisms. In addition, variants of isoprene synthase may possess improved activity such as improved enzymatic activity. In some aspects, an isoprene synthase variant has other improved properties, such as improved stability (e.g., thermo-stability), and/or improved solubility.

Standard methods can be used to determine whether a polypeptide has isoprene synthase polypeptide activity by measuring the ability of the polypeptide to convert DMAPP into isoprene in vitro, in a cell extract, or in vivo. Isoprene synthase polypeptide activity in the cell extract can be measured, for example, as described in Silver et al., J. Biol. Chem. 270:13010-13016, 1995.

In one aspect, DMAPP (Sigma) can be evaporated to dryness under a stream of nitrogen and rehydrated to a concentration of 100 mM in 100 mM potassium phosphate buffer pH 8.2 and stored at −200 C. To perform the assay, a solution of 5 μL of 1M MgCl₂, 1 mM (250 μg/ml) DMAPP, 65 μL of Plant Extract Buffer (PEB) (50 mM Tris-HCl, pH 8.0, 20 mM MgCl₂, 5% glycerol, and 2 mM DTT) can be added to 25 μL of cell extract in a 20 ml Headspace vial with a metal screw cap and teflon coated silicon septum (Agilent Technologies) and cultured at 370 C for 15 minutes with shaking. The reaction can be quenched by adding 200 μL of 250 mM EDTA and quantified by GC/MS.

In some aspects, the isoprene synthase polypeptide is a plant isoprene synthase polypeptide or a variant thereof. In some aspects, the isoprene synthase polypeptide is an isoprene synthase from Pueraria or a variant thereof. In some aspects, the isoprene synthase polypeptide is an isoprene synthase from Populus or a variant thereof. In some aspects, the isoprene synthase polypeptide is a poplar isoprene synthase polypeptide or a variant thereof. In some aspects, the isoprene synthase polypeptide is a kudzu isoprene synthase polypeptide or a variant thereof. In some aspects, the isoprene synthase polypeptide is a polypeptide from Pueraria or Populus or a hybrid, Populus alba×Populus tremula, or a variant thereof.

In some aspects, the isoprene synthase polypeptide or nucleic acid is from the family Fabaceae, such as the Faboideae subfamily. In some aspects, the isoprene synthase polypeptide or nucleic acid is a polypeptide or nucleic acid from Pueraria montana (kudzu) (Sharkey et al., Plant Physiology 137: 700-712, 2005), Pueraria lobata, poplar (such as Populus alba, Populus nigra, Populus trichocarpa, or Populus alba×tremula (CAC35696) (Miller et al., Planta 213: 483-487, 2001), aspen (such as Populus tremuloides) (Silver et al., JBC 270(22): 13010-1316, 1995), English Oak (Quercus robur) (Zimmer et al., WO 98/02550), or a variant thereof. In some aspects, the isoprene synthase polypeptide is an isoprene synthase from Pueraria montana, Pueraria lobata, Populus tremuloides, Populus alba, Populus nigra, or Populus trichocarpa or a variant thereof. In some aspects, the isoprene synthase polypeptide is an isoprene synthase from Populus alba or a variant thereof. In some aspects, the isoprene synthase polypeptide is an isoprene synthase from Clostridium or a variant thereof. In some aspects, the nucleic acid encoding the isoprene synthase (e.g., isoprene synthase from Populus alba or a variant thereof) is codon optimized.

In some aspects, the isoprene synthase nucleic acid or polypeptide is a naturally-occurring polypeptide or nucleic acid (e.g., naturally-occurring polypeptide or nucleic acid from Populus). In some aspects, the isoprene synthase nucleic acid or polypeptide is not a wild-type or naturally-occurring polypeptide or nucleic acid. In some aspects, the isoprene synthase nucleic acid or polypeptide is a variant of a wild-type or naturally-occurring polypeptide or nucleic acid (e.g., a variant of a wild-type or naturally-occurring polypeptide or nucleic acid from Populus).

In some aspects, the isoprene synthase polypeptide is a variant. In some aspects, the isoprene synthase polypeptide is a variant of a wild-type or naturally occurring isoprene synthase. In some aspects, the variant has improved activity such as improved catalytic activity compared to the wild-type or naturally occurring isoprene synthase. The increase in activity (e.g., catalytic activity) may be at least about any of 10%, 20%, 30%, 40%, 50%, 60%, 70%, 80%, 90%, or 95%. In some aspects, the increase in activity such as catalytic activity is at least about any of 1 fold, 2 folds, 5 folds, 10 folds, 20 folds, 30 folds, 40 folds, 50 folds, 75 folds, or 100 folds. In some aspects, the increase in activity such as catalytic activity is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the variant has improved solubility compared to the wild-type or naturally occurring isoprene synthase. The increase in solubility may be at least about any of 10%, 20%, 30%, 40%, 50%, 60%, 70%, 80%, 90%, or 95%. The increase in solubility may be at least about any of 1 fold, 2 folds, 5 folds, 10 folds, 20 folds, 30 folds, 40 folds, 50 folds, 75 folds, or 100 folds. In some aspects, the increase in solubility is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the isoprene synthase polypeptide is a variant of naturally occurring isoprene synthase and has improved stability (such as thermo-stability) compared to the naturally occurring isoprene synthase.

In some aspects, the variant has at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200% of the activity of a wild-type or naturally occurring isoprene synthase. The variant may share sequence similarity with a wild-type or naturally occurring isoprene synthase. In some aspects, a variant of a wild-type or naturally occurring isoprene synthase may have at least about any of 40%, 50%, 60%, 70%, 75%, 80%, 90%, 91%, 92%, 93%, 94%, 95%, 96%, 97%, 98%, 99%, 99.5%, or 99.9% amino acid sequence identity as that of the wild-type or naturally occurring isoprene synthase. In some aspects, a variant of a wild-type or naturally occurring isoprene synthase has any of about 70% to about 99.9%, about 75% to about 99%, about 80% to about 98%, about 85% to about 97%, or about 90% to about 95% amino acid sequence identity as that of the wild-type or naturally occurring isoprene synthase.

In some aspects, the variant comprises a mutation in the wild-type or naturally occurring isoprene synthase. In some aspects, the variant has at least one amino acid substitution, at least one amino acid insertion, and/or at least one amino acid deletion. In some aspects, the variant has at least one amino acid substitution. In some aspects, the number of differing amino acid residues between the variant and wild-type or naturally occurring isoprene synthase may be one or more, e.g. 1, 2, 3, 4, 5, 10, 15, 20, 30, 40, 50, or more amino acid residues. Naturally occurring isoprene synthases can include any isoprene synthases from plants, for example, kudzu isoprene synthases, poplar isoprene synthases, English oak isoprene synthases, and willow isoprene synthases. In some aspects, the variant is a variant of isoprene synthase from Populus alba. In some aspects, the variant of isoprene synthase from Populus alba has at least one amino acid substitution, at least one amino acid insertion, and/or at least one amino acid deletion. In some aspects, the variant is a truncated Populus alba isoprene synthase. In some aspects, the nucleic acid encoding variant (e.g., variant of isoprene synthase from Populus alba) is codon optimized (for example, codon optimized based on host cells where the heterologous isoprene synthase is expressed). For example, the nucleic acid encoding variant (e.g., variant of isoprene synthase from Populus alba) may be codon optimized for Clostridium acetobutylicum and/or Clostridium kluyveri.

The isoprene synthase polypeptide provided herein may be any of the isoprene synthases or isoprene synthase variants described in WO 2009/132220, WO 2010/124146, and U.S. Patent Application Publication No.: 2010/0086978, U.S. Pat. No. 8,173,410, and U.S. patent application Ser. No. 13/283,564 (US 2013/0045891), the contents of which are expressly incorporated herein by reference in their entirety with respect to the isoprene synthases and isoprene synthase variants.

In some aspects, the isoprene synthase polypeptide is less susceptible to degradation (e.g., degradation by protease(s)) in the cells during culturing. In some aspects, the isoprene synthase polypeptide is less susceptible to degradation in the cells when using isoprene synthase polypeptide (e.g., a variant) having more resistance to degradation by protease(s) in the cells. In some aspects, the isoprene synthase polypeptide (e.g., a variant) has mutation(s) in the wild-type or naturally occurring isoprene synthase, and wherein the isoprene synthase polypeptide having mutation(s) is more resistant to degradation by protease(s). In some aspects the isoprene synthase polypeptide (e.g., a variant) has a heterologous oligopeptide in the N-terminal end (N-terminus), wherein the isoprene synthase polypeptide having a heterologous oligopeptide in the N-terminal end is more resistant to degradation by protease(s). The heterologous oligopeptide is a sequence of amino acids that is not present in the wild-type or naturally occurring isoprene synthase in the N-terminal end. In one aspect the heterologous oligopeptide is a his-tag. In some aspects, the degradation of isoprene synthase polypeptide in the cells when using these variant isoprene synthase polypeptides is less compared to the degradation of isoprene synthase polypeptide in the cells when not using these variant isoprene synthase polypeptides. In some aspects, the degradation of isoprene synthase polypeptide in the cells when using such isoprene synthase polypeptide is less compared to the degradation of isoprene synthase polypeptide in the cells when using a wild-type or naturally occurring isoprene synthase.

A variant of a wild-type or naturally occurring isoprene synthase may be more resistant to cleavage by a protease in the cells compared to the wild-type or naturally occurring isoprene synthase. A variant may have increased resistance to cleavage by a protease in the cells. The degradation of the variant isoprene synthase polypeptide expressed in the cells may be reduced by at least about any of 10%, 20%, 30%, 40%, 50%, 60%, 70%, 80%, 90%, or 95% compared to a wild-type or naturally occurring isoprene synthase. The degradation of the variant isoprene synthase polypeptide expressed in the cells may be reduced by at least about any of 1 fold, 2 folds, 5 folds, 10 folds, 20 folds, 30 folds, 40 folds, 50 folds, 75 folds, or 100 folds compared to a wild-type or naturally occurring isoprene synthase. In some aspects, the variant has increased resistance to cleavage by a protease in the cells, whereby the degradation of the variant isoprene synthase polypeptide expressed in the cells is reduced by about 10% to about 100 folds (e.g., about 20% to about 100 folds, about 50% to about 50 folds, about 1 fold to about 25 folds, about 2 folds to about 20 folds, or about 5 folds to about 20 folds) compared to naturally occurring isoprene synthase. In some aspects, the isoprene synthase polypeptide is resistant (e.g., substantially resistant) to cleavage by a protease in the cell. In some aspects, the protease is a protease that cleaves isoprene synthase. In some aspects, the cells are deficient in protease (e.g., a protease that cleaves isoprene synthase that is expressed in the cells). In some aspects, the cells are deficient in protease such that the isoprene synthase polypeptide is not degraded or more resistant to degradation compared to cells that are not deficient in the protease.

Any one of the promoters described herein that is active or functional in the cells in which expression is desired (e.g., promoters described herein and identified in the Examples of the present disclosure including inducible promoters and constitutive promoters) may be used to drive expression of any of the isoprene synthases described herein. Any plasmid that can replicate in the present cells, such as those described in Heap et al. (Journal of Microbiological Methods 78 (2009) p79-85), may be used to express any of the isoprene synthases described herein. In one aspect, a plasmid having a gram positive origin of replication isolated from a plasmid isolated from Clostridium butyricum (SEQ ID NO:1) or from a plasmid isolated from Clostridium botulinum is used. These origins of replication are present in constructed plasmids pCB 102 and pBP 1, respectively. In one aspect the origin of replication has the sequence of SEQ ID NO:1 or of SEQ ID NO:62. Any one of the strains in which isoprene synthase is not degraded or the degradation of isoprene synthase is reduced (including the strain(s) identified in the Examples of the present disclosure) may be used for expressing isoprene synthase.

Suitable isoprene synthases include, but are not limited to, those identified by Genbank Accession Nos. AY341431, AY316691, AY279379, AJ457070, and AY182241. Types of isoprene synthases which can be used in any one of the compositions or methods including methods of making microorganisms encoding isoprene synthase described herein are also described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/124146, WO2010/078457, and WO2010/148256.

### MVA Pathway

In some aspects of the invention, the cells described in any of the compositions or methods described herein comprise one or more nucleic acids encoding mevalonate (MVA) pathway polypeptide(s). In some aspects, the MVA pathway polypeptide is an endogenous polypeptide. In some aspects, the cells comprise one or more additional copies of an endogenous nucleic acid encoding an MVA pathway polypeptide. In some aspects, the endogenous nucleic acid encoding an MVA pathway polypeptide is operably linked to a constitutive promoter. In some aspects, the endogenous nucleic acid encoding an MVA pathway polypeptide is operably linked to a constitutive promoter. In some aspects, the endogenous nucleic acid encoding an MVA pathway polypeptide is operably linked to a strong promoter. In a particular aspect, the cells are engineered to over-express the endogenous MVA pathway polypeptide relative to wild-type cells. In some aspects, the endogenous nucleic acid encoding an MVA pathway polypeptide is operably linked to a weak promoter. In some aspects, the MVA pathway polypeptide is a polypeptide from Saccharomyces cerevisiae, Enterococcus faecalis, or Methanosarcina mazei.

In some aspects, the MVA pathway polypeptide is a heterologous polypeptide. In some aspects, the cells comprise more than one copy of a heterologous nucleic acid encoding an MVA pathway polypeptide. In some aspects, the heterologous nucleic acid encoding an MVA pathway polypeptide is operably linked to a constitutive promoter. In some aspects, the heterologous nucleic acid encoding an MVA pathway polypeptide is operably linked to an inducible promoter. In some aspects, the heterologous nucleic acid encoding an MVA pathway polypeptide is operably linked to a strong promoter. In some aspects, the heterologous nucleic acid encoding an MVA pathway polypeptide is operably linked to a weak promoter.

The nucleic acids encoding MVA pathway polypeptide(s) may be integrated into a genome of the cells. The nucleic acids encoding MVA pathway polypeptide(s) may be stably expressed in the cells. The nucleic acids encoding MVA pathway polypeptide(s) may be on a vector.

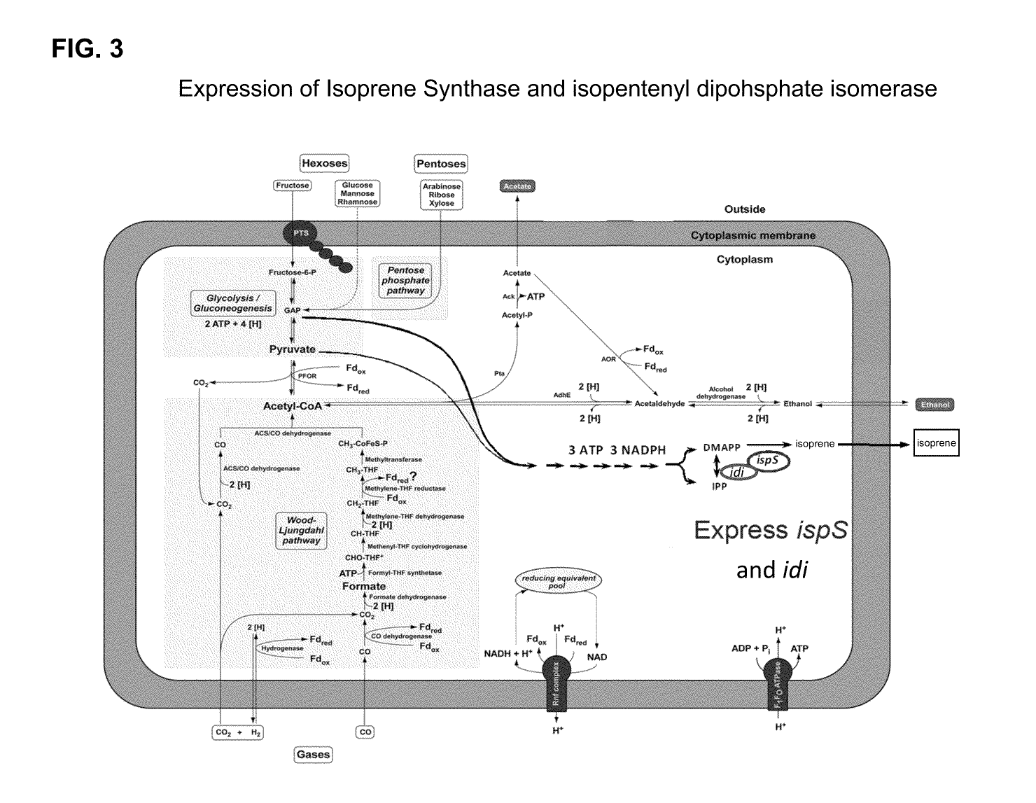

The upper mevalonate biosynthetic pathway comprises two genes encoding three enzymatic activities: the mvaE gene encoding a protein with the enzymatic activities of both acetyl-CoA acetyltransferase and 3-hydroxy-3-methylglutaryl-CoA (HMG-CoA) reductase, the first and third proteins in the pathway, and the mvaS gene encoding second enzyme in the pathway, HMG-CoA synthase. The lower mevalonate biosynthetic pathway comprises mevalonate kinase (MVK), phosphomevalonate kinase (PMK), and diphosphomevalonte decarboxylase (MVD). In some aspects, the lower MVA pathway can further comprise isopentenyl diphosphate isomerase (IDI). Cells provided herein may comprise at least one nucleic acid encoding isoprene synthase, one or more upper MVA pathway polypeptides, and/or one or more lower MVA pathway polypeptides. For example FIG. 3 shows various pathways in obligate anaerobes expressing heterologous isoprene synthase and a heterologous isopentenyl diphosphate isomerase. When more than one nucleic acid encoding a polypeptide is introduced into a cell, the nucleic acids may be operably linked to a promoter separately or in an operon. For example, coding regions for ispS and IDI may be in one operon expressed from the same promoter.

Exemplary MVA pathway polypeptides are also provided below: acetyl-CoA acetyltransferase (AA-CoA thiolase) polypeptides, 3-hydroxy-3-methylglutaryl-CoA synthase (HMG-CoA synthase) polypeptides, 3-hydroxy-3-methylglutaryl-CoA reductase (HMG-CoA reductase) polypeptides, mevalonate kinase (MVK) polypeptides, phosphomevalonate kinase (PMK) polypeptides, diphosphomevalonte decarboxylase (MVD) polypeptides, phosphomevalonate decarboxylase (PMDC) polypeptides, isopentenyl phosphate kinase (IPK) polypeptides, IPP isomerase polypeptides, IDI polypeptides, and polypeptides (e.g., fusion polypeptides) having an activity of two or more MVA pathway polypeptides. In particular, MVA pathway polypeptides include polypeptides, fragments of polypeptides, peptides, and fusions polypeptides that have at least one activity of an MVA pathway polypeptide. Exemplary MVA pathway nucleic acids include nucleic acids that encode a polypeptide, fragment of a polypeptide, peptide, or fusion polypeptide that has at least one activity of an MVA pathway polypeptide. Exemplary MVA pathway polypeptides and nucleic acids include naturally-occurring polypeptides and nucleic acids from any of the source organisms described herein. In addition, variants of MVA pathway polypeptide that confer the result of better isoprene production can also be used as well.

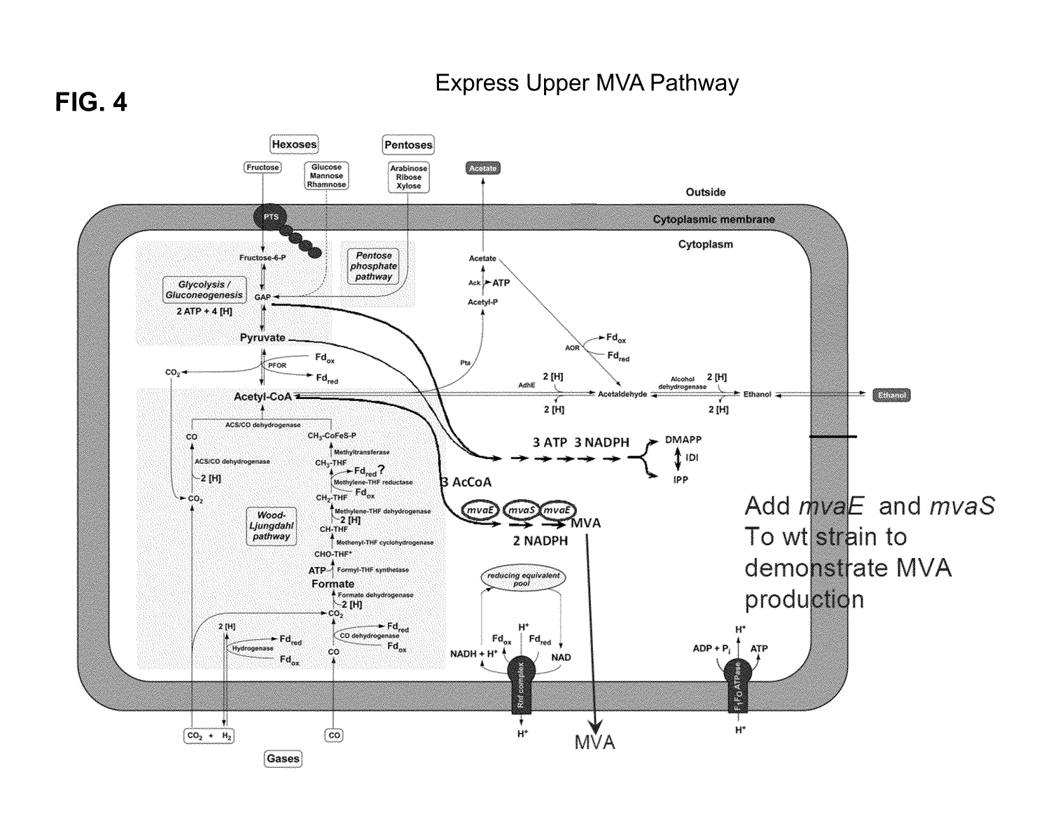

In some aspects, any of the cells described herein may further comprise the upper MVA pathway, which includes AA-CoA thiolase, HMG-CoA synthase, and HMG-CoA reductase nucleic acids. FIG. 4 shows a schematic representation of an obligate anaerobe engineered with mvaE and mvaS to express upper MVA pathway. When more than one nucleic acid encoding a polypeptide is introduced into a cell, the nucleic acids may be operably linked to a promoter separately or in an operon. For example, coding regions for MvaE and MvaS may be in one operon expressed from the same promoter.

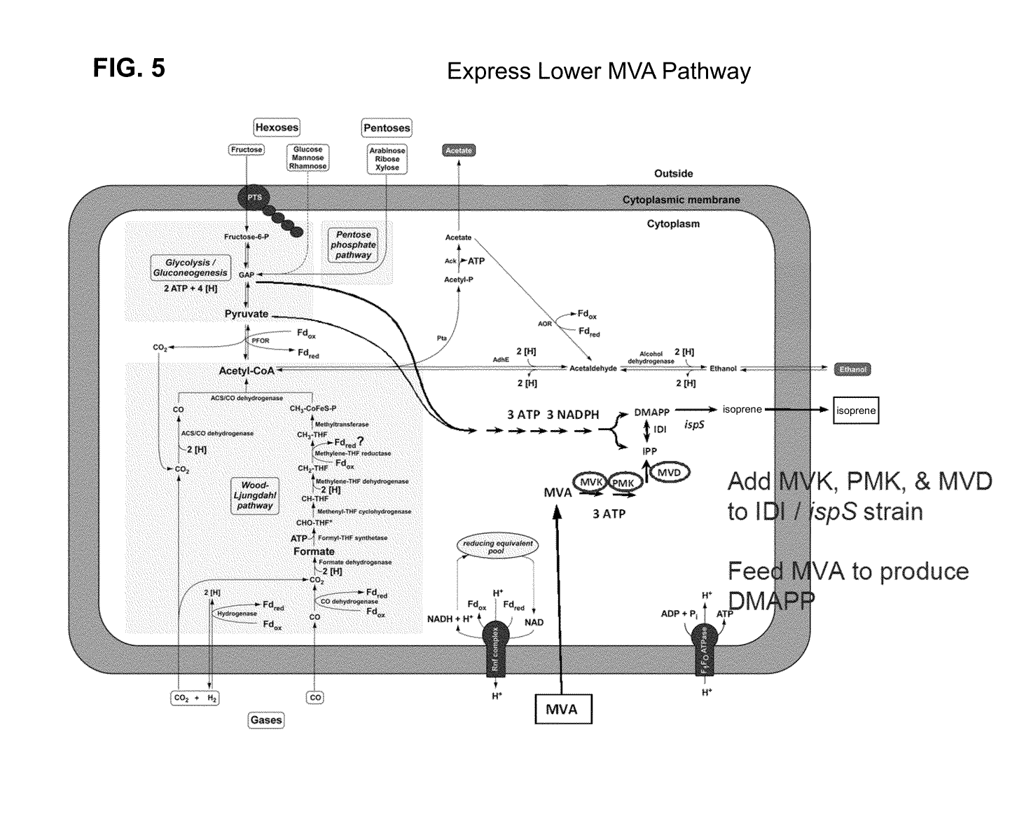

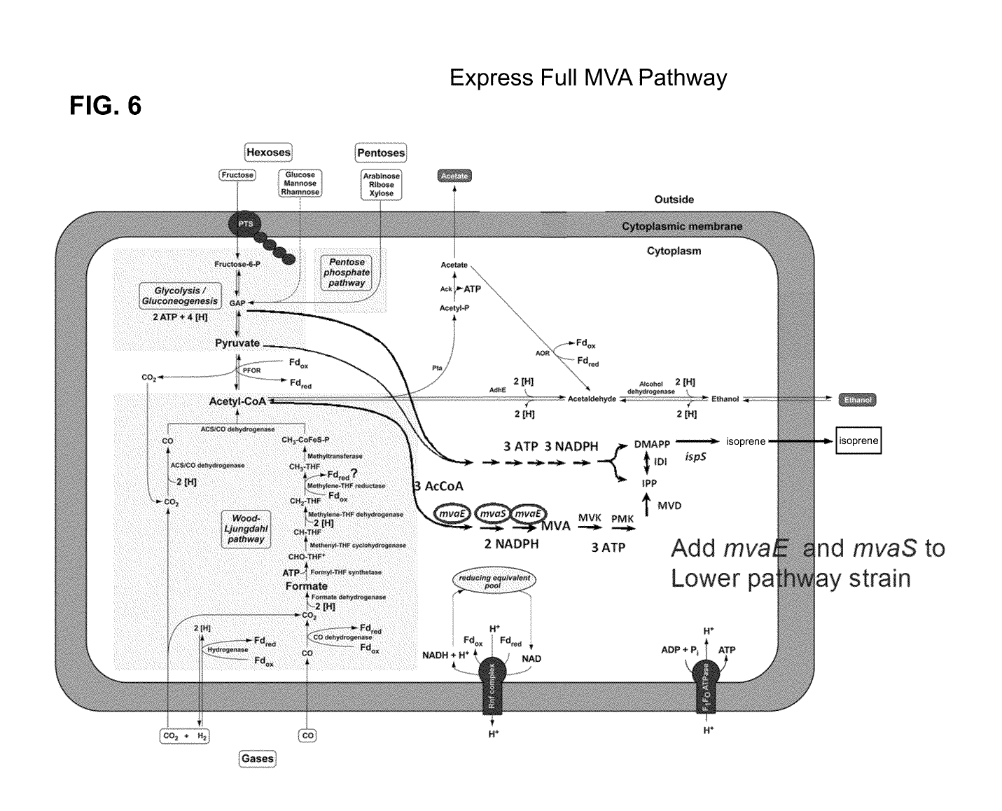

In some aspects, any of the cells described herein may further comprise the lower MVA pathway, which includes MVK, PMK, and MVD nucleic acids. In some aspects, any of the cells described herein may further comprise an IDI nucleic acid. FIG. 5 shows a schematic representation of expressing lower MVA pathway in an obligate anaerobe including expressing (a) a heterologous MVK polypeptide, (b) a heterologous PMK polypeptide, and (c) a heterologous MVD polypeptide in the cells expressing heterologous IDI polypeptide and heterologous IspS polypeptide for the purpose of increasing isoprene production. In some aspects, any of the cells described herein may further comprise the entire MVA pathway, which includes AA-CoA thiolase, HMG-CoA synthase, HMG-CoA reductase, MVK, PMK, MVD, and IDI nucleic acids. In some aspects, the cells comprise nucleic acids encoding at least two (at least any of 3, 4, 5, or 6) MVA pathway polypeptides. FIG. 6 shows a schematic representation of expressing entire MVA pathway in an obligate anaerobe by introducing mvaE and mvaS in the cells expressing (a) a heterologous MVK polypeptide, (b) a heterologous PMK polypeptide, (c) a heterologous MVD polypeptide, (d) a heterologous IDI polypeptide, and (e) a heterologous IspS polypeptide for the purpose of increasing isoprene production.

Any one of the cells described herein may comprise IDI nucleic acid(s) (e.g., endogenous or heterologous nucleic acid(s) encoding IDI). Isopentenyl diphosphate isomerase polypeptides (isopentenyl-diphosphate delta-isomerase or IDI) catalyzes the interconversion of isopentenyl diphosphate (IPP) and dimethylallyl diphosphate (DMAPP) (e.g., converting IPP into DMAPP and/or converting DMAPP into IPP). Exemplary IDI polypeptides include polypeptides, fragments of polypeptides, peptides, and fusions polypeptides that have at least one activity of an IDI polypeptide. Standard methods (such as those described herein) can be used to determine whether a polypeptide has IDI polypeptide activity by measuring the ability of the polypeptide to interconvert IPP and DMAPP in vitro, in a cell extract, or in vivo. Exemplary IDI nucleic acids include nucleic acids that encode a polypeptide, fragment of a polypeptide, peptide, or fusion polypeptide that has at least one activity of an IDI polypeptide. Exemplary IDI polypeptides and nucleic acids include naturally-occurring polypeptides and nucleic acids from any of the source organisms described herein as well as mutant polypeptides and nucleic acids derived from any of the source organisms described herein.

In some aspects, the MVA pathway polypeptide is a polypeptide from Saccharomyces cerevisiae, Enterococcus faecalis, or Methanosarcina mazei. In some aspects, the MVK polypeptide is selected from the group consisting of Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, Streptomyces mevalonate kinase polypeptide, Streptomyces CL190 mevalonate kinase polypeptide, and Methanosarcina mazei mevalonate kinase polypeptide.

Any one of the promoters described herein (e.g., promoters described herein and identified in the Examples of the present disclosure including inducible promoters and constitutive promoters) may be used to drive expression of any of the MVA polypeptides described herein. Any one of the dual plasmid system identified in the Examples of the present disclosure may be used to express any of the MVA polypeptides described herein.

### Acetoacetyl-CoA Synthase

In one aspect, any of the cells described herein can contain one or more heterologous nucleic acid(s) encoding an acetoacetyl-CoA synthase polypeptide. The acetoacetyl-CoA synthase gene (a.k.a. nphT7) is a gene encoding an enzyme having the activity of synthesizing acetoacetyl-CoA from malonyl-CoA and acetyl-CoA and having minimal activity (e.g., no activity) of synthesizing acetoacetyl-CoA from two acetyl-CoA molecules. See, e.g., Okamura et al., PNAS Vol 107, No. 25, pp. 11265-11270 (2010), the contents of which are expressly incorporated herein for teaching about nphT7. An acetoacetyl-CoA synthase gene from an actinomycete of the genus Streptomyces CL190 strain was described in Japanese Patent Publication (Kokai) No. 2008-61506 A and U.S. Patent Application Publication No. 2010/0285549, the disclosure of each of which are incorporated by reference herein. Acetoacetyl-CoA synthase can also be referred to as acetyl CoA:malonyl CoA acyltransferase. A representative acetoacetyl-CoA synthase (or acetyl CoA:malonyl CoA acyltransferase) that can be used is Genbank AB540131.1.

In one aspect, acetoacetyl-CoA synthase of the present invention synthesizes acetoacetyl-CoA from malonyl-CoA and acetyl-CoA via an irreversible reaction. The use of acetoacetyl-CoA synthase to generate acetoacetyl-CoA provides an additional advantage in that this reaction is irreversible while acetoacetyl-CoA thiolase enzyme's action of synthesizing acetoacetyl-CoA from two acetyl-CoA molecules is reversible. Consequently, the use of acetoacetyl-CoA synthase to synthesize acetoacetyl-CoA from malonyl-CoA and acetyl-CoA can result in significant improvement in productivity for isoprene compared with using thiolase to generate the end same product.

Furthermore, the use of acetoacetyl-CoA synthase to produce isoprene provides another advantage in that acetoacetyl-CoA synthase can convert malonyl CoA to acetyl CoA via decarboxylation of the malonyl CoA. Thus, stores of starting substrate are not limited by the starting amounts of acetyl CoA. The synthesis of acetoacetyl-CoA by acetoacetyl-CoA synthase can still occur when the starting substrate is only malonyl-CoA. In one aspect, the pool of starting malonyl-CoA is increased by using host strains that have more malonyl-CoA. Such increased pools can be naturally occurring or be engineered by molecular manipulation. See, for example Fowler, et al., Applied and Environmental Microbiology, Vol. 75, No. 18, pp. 5831-5839 (2009).

In any of the aspects or embodiments described herein, an enzyme that has the ability to synthesize acetoacetyl-CoA from malonyl-CoA and acetyl-CoA can be used. Non-limiting examples of such an enzyme are described herein. In certain embodiments described herein, an acetoacetyl-CoA synthase gene derived from an actinomycete of the genus Streptomyces having the activity of synthesizing acetoacetyl-CoA from malonyl-CoA and acetyl-CoA can be used.

An example of such an acetoacetyl-CoA synthase gene is the gene encoding a protein having the amino acid sequence of SEQ ID NO:2. Such a protein having the amino acid sequence of SEQ ID NO:2 corresponds to an acetoacetyl-CoA synthase having activity of synthesizing acetoacetyl-CoA from malonyl-CoA and acetyl-CoA and having no activity of synthesizing acetoacetyl-CoA from two acetyl-CoA molecules.

In one embodiment, the gene encoding a protein having the amino acid sequence of SEQ ID NO:1 can be obtained by a nucleic acid amplification method (e.g., PCR) with the use of genomic DNA obtained from an actinomycete of the Streptomyces sp. CL 190 strain as a template and a pair of primers that can be designed with reference to Japanese Patent Publication (Kokai) No. 2008-61506 A.

As described herein, an acetoacetyl-CoA synthase gene for use in the present invention is not limited to a gene encoding a protein having the amino acid sequence of SEQ ID NO:2 from an actinomycete of the Streptomyces sp. CL190 strain. Any gene encoding a protein having the ability to synthesize acetoacetyl-CoA from malonyl-CoA and acetyl-CoA and which does not synthesize acetoacetyl-CoA from two acetyl-CoA molecules can be used in the presently described methods. In certain embodiments, the acetoacetyl-CoA synthase gene can be a gene encoding a protein having an amino acid sequence with high similarity or substantially identical to the amino acid sequence of SEQ ID NO:2 and having the function of synthesizing acetoacetyl-CoA from malonyl-CoA and acetyl-CoA. The expression “highly similar” or “substantially identical” refers to, for example, at least about 80% identity, at least about 85%, at least about 90%, at least about 91%, at least about 92%, at least about 93%, at least about 94%, at least about 95%, at least about 96%, at least about 97%, at least about 98%, and at least about 99% identity. As used above, the identity value corresponds to the percentage of identity between amino acid residues in a different amino acid sequence and the amino acid sequence of SEQ ID NO:2, which is calculated by performing alignment of the amino acid sequence of SEQ ID NO:2 and the different amino acid sequence with the use of a program for searching for a sequence similarity.

In other embodiments, the acetoacetyl-CoA synthase gene may be a gene encoding a protein having an amino acid sequence derived from the amino acid sequence of SEQ ID NO:2 by substitution, deletion, addition, or insertion of 1 or more amino acid(s) and having the function of synthesizing acetoacetyl-CoA from malonyl-CoA and acetyl-CoA. Herein, the expression “more amino acids” refers to, for example, 2 to 30 amino acids, preferably 2 to 20 amino acids, more preferably 2 to 10 amino acids, and most preferably 2 to 5 amino acids.

In still other embodiments, the acetoacetyl-CoA synthase gene may consist of a polynucleotide capable of hybridizing to a portion or the entirety of a polynucleotide having a nucleotide sequence complementary to the nucleotide sequence encoding the amino acid sequence of SEQ ID NO:2 under stringent conditions and capable of encoding a protein having the function of synthesizing acetoacetyl-CoA from malonyl-CoA and acetyl-CoA. Herein, hybridization under stringent conditions corresponds to maintenance of binding under conditions of washing at 60° C. 2×SSC. Hybridization can be carried out by conventionally known methods such as the method described in J. Sambrook et al. Molecular Cloning, A Laboratory Manual, 3rd Ed., Cold Spring Harbor Laboratory (2001).

As described herein, a gene encoding an acetoacetyl-CoA synthase having an amino acid sequence that differs from the amino acid sequence of SEQ ID NO:2 can be isolated from potentially any organism, for example, an actinomycete that is not obtained from the Streptomyces sp. CL190 strain. In addition, acetoacetyl-CoA synthase genes for use herein can be obtained by modifying a polynucleotide encoding the amino acid sequence of SEQ ID NO:2 by a method known in the art. Mutagenesis of a nucleotide sequence can be carried out by a known method such as the Kunkel method or the gapped duplex method or by a method similar to either thereof. For instance, mutagenesis may be carried out with the use of a mutagenesis kit (e.g., product names; Mutant-K and Mutant-G (TAKARA Bio)) for site-specific mutagenesis, product name; an LA PCR in vitro Mutagenesis series kit (TAKARA Bio), and the like.

The activity of an acetoacetyl-CoA synthase having an amino acid sequence that differs from the amino acid sequence of SEQ ID NO:2 can be evaluated as described below. Specifically, a gene encoding a protein to be evaluated is first introduced into a host cell such that the gene can be expressed therein, followed by purification of the protein by a technique such as chromatography. Malonyl-CoA and acetyl-CoA are added as substrates to a buffer containing the obtained protein to be evaluated, followed by, for example, incubation at a desired temperature (e.g., 10° C. to 60° C.). After the completion of reaction, the amount of substrate lost and/or the amount of product (acetoacetyl-CoA) produced are determined. Thus, it is possible to evaluate whether or not the protein being tested has the function of synthesizing acetoacetyl-CoA from malonyl-CoA and acetyl-CoA and to evaluate the degree of synthesis. In such case, it is possible to examine whether or not the protein has the activity of synthesizing acetoacetyl-CoA from two acetyl-CoA molecules by adding acetyl-CoA alone as a substrate to a buffer containing the obtained protein to be evaluated and determining the amount of substrate lost and/or the amount of product produced in a similar manner.

### DXP Pathway

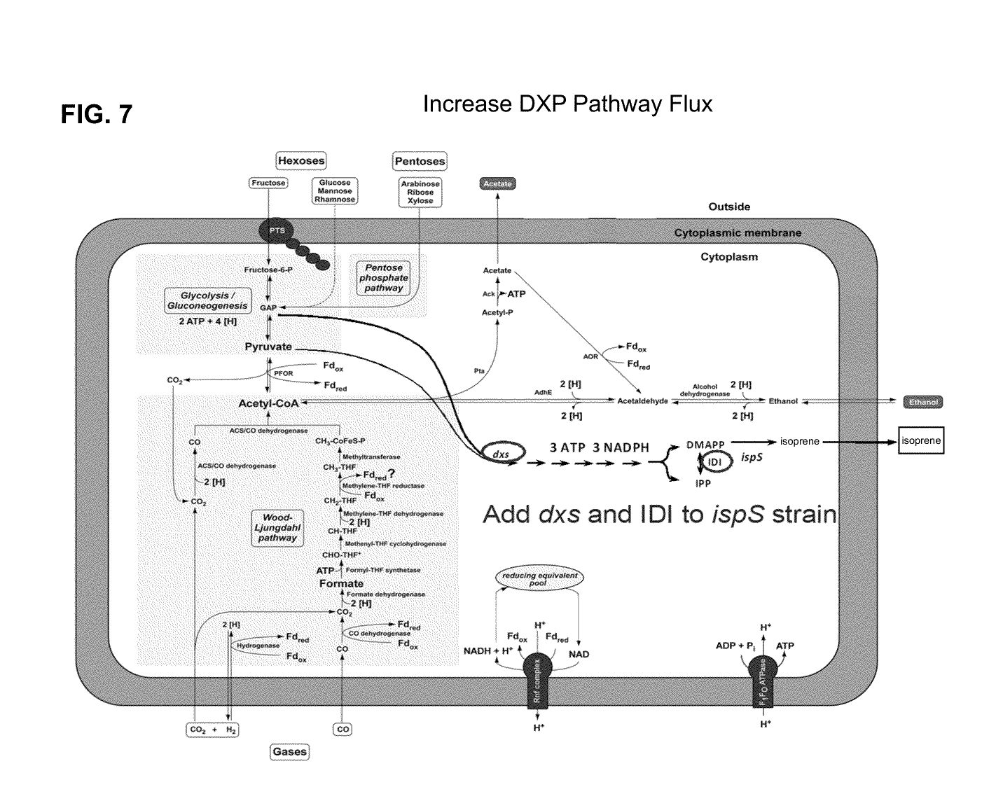

Any of the cells described herein may further comprise one or more nucleic acids encoding DXP pathway polypeptide(s). DXP pathway polypeptides include, but are not limited to any of the following polypeptides: DXS polypeptides, DXR polypeptides, MCT polypeptides, CMK polypeptides, MCS polypeptides, HDS polypeptides, HDR polypeptides, and polypeptides (e.g., fusion polypeptides) having an activity of one, two, or more of the DXP pathway polypeptides. Exemplary DXP pathway polypeptides and nucleic acids and methods of measuring DXP pathway polypeptide activity are described in more detail in International Publication No.: WO 2010/148150. In some aspects, the DXP pathway polypeptides comprise DXS. In some aspects, the DXS polypeptide is a yeast DXS polypeptide. The nucleic acids encoding DXP pathway polypeptide(s) may be endogenous copy of nucleic acid. The nucleic acids encoding DXP pathway polypeptide(s) may be heterologous. The DXP pathway polypeptides may be from yeast. The nucleic acids encoding DXP pathway polypeptide(s) may be over-expressed. The over-expressed nucleic acid may be cloned into a multicopy plasmid. The nucleic acids encoding DXP pathway polypeptide(s) may be integrated into a genome of the cells. The nucleic acids encoding DXP pathway polypeptide(s) may be stably expressed in the cells. The nucleic acids encoding DXP pathway polypeptide(s) may be on a vector. In some aspects, the cells further comprise one or more nucleic acids encoding an IDI polypeptide and a DXS polypeptide or other DXP pathway polypeptides. In some aspects, one nucleic acid encodes the isoprene synthase polypeptide, IDI polypeptide, and DXS polypeptide or other DXP pathway polypeptides. FIG. 7 shows a schematic representation of an obligate anaerobe expressing (a) a heterologous IspS polypeptide, (b) a heterologous DXS polypeptide, and (c) a heterologous IDI polypeptide to increase DXP pathway flux and isoprene production.

In some aspects, one plasmid encodes the isoprene synthase polypeptide, IDI polypeptide, and DXS polypeptide or other DXP pathway polypeptides. In some aspects, multiple plasmids encode the isoprene synthase polypeptide, IDI polypeptide, and DXS polypeptide or other DXP pathway polypeptides.

Any one of the promoters described herein (e.g., promoters described herein and identified in the Examples of the present disclosure including inducible promoters and constitutive promoters) may be used to drive expression of any of the DXP polypeptides described herein. In some aspects, the nucleic acid encoding a DXP pathway polypeptide is operably linked to a constitutive promoter. In some aspects, the nucleic acid encoding a DXP pathway polypeptide is operably linked to an inducible promoter. In some aspects, the nucleic acid encoding a DXP pathway polypeptide is operably linked to a strong promoter. In a particular aspect, the cells are engineered to over-express the endogenous DXP pathway polypeptide relative to wild-type cells. In some aspects, the nucleic acid encoding a DXP pathway polypeptide is operably linked to a weak promoter. Any one of the dual plasmid system identified in the Examples of the present disclosure may be used to express any of the DXP polypeptides described herein.

In particular, DXS (1-deoxy-D-xylulose-5-phosphate synthase; EC 2.2.1.7) polypeptides convert pyruvate and D-glyceraldehyde 3-phosphate into 1-deoxy-d-xylulose 5-phosphate (DXP). Standard methods can be used to determine whether a polypeptide has DXS polypeptide activity by measuring the ability of the polypeptide to convert pyruvate and D-glyceraldehyde 3-phosphate in vitro, in a cell extract, or in vivo.

DXR (1-deoxy-d-xylulose 5-phosphate reductoisomerase; EC 1.1.1.267) polypeptides convert 1-deoxy-d-xylulose 5-phosphate (DXP) into 2-C-methyl-D-erythritol 4-phosphate (MEP). Standard methods can be used to determine whether a polypeptide has DXR polypeptides activity by measuring the ability of the polypeptide to convert DXP in vitro, in a cell extract, or in vivo.

MCT polypeptides convert 2-C-methyl-D-erythritol 4-phosphate (MEP) into 4-(cytidine 5′-diphospho)-2-methyl-D-erythritol (CDP-ME). Standard methods can be used to determine whether a polypeptide has MCT polypeptides activity by measuring the ability of the polypeptide to convert MEP in vitro, in a cell extract, or in vivo.

CMK polypeptides convert 4-(cytidine 5′-diphospho)-2-C-methyl-D-erythritol (CDP-ME) into 2-phospho-4-(cytidine 5′-diphospho)-2-C-methyl-D-erythritol (CDP-MEP). Standard methods can be used to determine whether a polypeptide has CMK polypeptides activity by measuring the ability of the polypeptide to convert CDP-ME in vitro, in a cell extract, or in vivo.

MCS polypeptides convert 2-phospho-4-(cytidine 5′-diphospho)-2-C-methyl-D-erythritol (CDP-MEP) into 2-C-methyl-D-erythritol 2, 4-cyclodiphosphate (ME-CPP or cMEPP). Standard methods can be used to determine whether a polypeptide has MCS polypeptides activity by measuring the ability of the polypeptide to convert CDP-MEP in vitro, in a cell extract, or in vivo.

HDS polypeptides convert 2-C-methyl-D-erythritol 2,4-cyclodiphosphate into (E)-4-hydroxy-3-methylbut-2-en-1-yl diphosphate (HMBPP or HDMAPP). Standard methods can be used to determine whether a polypeptide has HDS polypeptides activity by measuring the ability of the polypeptide to convert ME-CPP in vitro, in a cell extract, or in vivo.

HDR polypeptides convert (E)-4-hydroxy-3-methylbut-2-en-1-yl diphosphate into isopentenyl diphosphate (IPP) and dimethylallyl diphosphate (DMAPP). Standard methods can be used to determine whether a polypeptide has HDR polypeptides activity by measuring the ability of the polypeptide to convert HMBPP in vitro, in a cell extract, or in vivo.

In some aspects, the cells further comprise one or more nucleic acids encoding IDI. In some aspects, the IDI polypeptide is a yeast IDI polypeptide. In some aspects, the cells comprise one or more heterologous nucleic acids encoding an isoprene synthase polypeptide, one or more nucleic acids encoding DXS, and/or one or more nucleic acids encoding IDI. In some aspects, the cells comprise one or more heterologous nucleic acids encoding an isoprene synthase polypeptide, one or more nucleic acids encoding acetyl-CoA acetyltransferase, one or more nucleic acids encoding 3-hydroxy-3-methylglutaryl-CoA (HMG-CoA) reductase, and/or one or more nucleic acids encoding HMG-CoA synthase. In some aspects, the cells comprise one or more heterologous nucleic acids encoding an isoprene synthase polypeptide, one or more nucleic acids encoding MVK, one or more nucleic acids encoding PMK, one or more nucleic acids encoding MVD, and/or one or more nucleic acids encoding IDI. In some aspects, the cells comprise one or more heterologous nucleic acids encoding an isoprene synthase polypeptide, one or more nucleic acids encoding MVK, one or more nucleic acids encoding PMK, one or more nucleic acids encoding MVD, one or more nucleic acids encoding IDI, one or more nucleic acids encoding acetyl-CoA acetyltransferase, one or more nucleic acids encoding 3-hydroxy-3-methylglutaryl-CoA (HMG-CoA) reductase, and/or one or more nucleic acids encoding HMG-CoA synthase. In some aspects, the cells comprise one or more heterologous nucleic acids encoding an isoprene synthase polypeptide, one or more nucleic acids encoding MVK, one or more nucleic acids encoding PMK, one or more nucleic acids encoding MVD, one or more nucleic acids encoding acetyl-CoA acetyltransferase, one or more nucleic acids encoding 3-hydroxy-3-methylglutaryl-CoA (HMG-CoA) reductase, one or more nucleic acids encoding HMG-CoA synthase, one or more nucleic acids encoding DXS, and/or one or more nucleic acids encoding IDI. In some aspects, the cells comprise (a) one or more heterologous nucleic acids encoding an isoprene synthase polypeptide, (b) one or more nucleic acids encoding an isopentenyl-diphosphate delta-isomerase (IDI) polypeptide, and (c) (i) a 1-Deoxyxylulose-5-phosphate synthase (DXS) polypeptide and/or (ii) one or more MVA pathway polypeptides (e.g., acetyl-CoA acetyltransferase, 3-hydroxy-3-methylglutaryl-CoA (HMG-CoA) reductase, HMG-CoA synthase, MVK, PMK, and/or MVD).

Types of MVA pathway polypeptides and/or DXP pathway polypeptides which can be used and methods of making microorganisms (e.g., facultative anaerobes such as E. coli) encoding MVA pathway polypeptides and/or DXP pathway polypeptides are also described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/078457, and WO2010/148256, the contents of which are incorporated herein by reference in their entirety with respect to MVA pathway polypeptides and DXP pathway polypeptides.

### Phosphoketotase Shunt

Theoretically, three molecules of acetyl-CoA can be derived from a single molecule of glucose in a balanced reaction. However, organisms typically produce only up to two molecules of acetyl-CoA, with the remainder mass being lost as CO₂. The release of CO₂ occurs during the formation of acetyl-CoA from pyruvate, a reaction catalyzed by pyruvate dehydrogenase. The loss of one carbon atom results in decreased production yields of mevalonate, isoprenoid precursors, isoprene, and isoprenoid molecules. An exception to this reaction loss is the Wood-Ljungdahl pathway, which relies on carbon monoxide dehydrogenase and acetyl-CoA synthase enzymes to reduce the carbon dioxide to acetyl-CoA in anaerobic acetogens.

An alternate metabolic process exists which can potentially produce three molecules of acetyl-CoA from one molecule of glucose using a pathway which does not rely on the Wood-Ljungdahl pathway enzymes. This alternate process makes use of a phosphoketolase enzyme found in certain organisms, particularly among Bifidobacteria [see, for example, Biology of the Prokaryotes (ed. Lengeler, Drews and Schlegel); Blackwell Science, New York, 1999, p. 299-301; Meile et al., J. of Bacteriology, 2001, 183:9, 2929-36; Jeong et al., J. Microbiol. Biotechnol., 2007, 17:5, 822-829]. Phosphoketolase enzymes allow for formation of acetyl-CoA (via acetyl-phosphate) from xylulose 5-phosphate or fructose 6-phosphate rather than through oxidation of pyruvate as in typical metabolism.

Phosphoketolases have been classified into two types based on their substrate preference: xylulose-5-phosphate (X5P) phosphoketolases, which only act on X5P, and X5P/fructose-6-phosphate (F6P) phosphoketolases, which can act on both X5P and F6P (Suzuki et al., Acta Cryst. F66, 2010, 66:8, 941-43). Phosphoketolases catalyze the cleavage of X5P or F6P utilizing inorganic phosphate (Pᵢ) to produce acetyl phosphate (acetyl-P), H₂O and glyceraldehyde 3-phosphate or erythrose 4-phosphate. The high-energy metabolite acetyl-P is subsequently converted to acetic acid by acetate kinase to produce ATP from ADP in the pathway. In addition to acetyl-phosphate, the glyceraldehyde 3-phosphate produced from the enzymatic reaction can be recycled through manipulated metabolic pathways so that the maximum yield of 3 acetyl-CoA per glucose can be achieved. Significantly, acetyl-CoA production by phosphoketolase eliminates the loss of carbon (e.g. CO₂) as observed from pyruvate dehydrogenase mediated reactions.

As further detailed herein, phosphoketolases can also act upon sedoheptulose-7-phosphate to convert it to ribose-5-phosphate and acetyl phosphate. A non-limiting example of such a phosphoketolase is Bifidobacterium longum phosphoketolase, which has catalytic activity with sedoheptulose-7-phosphate.

Methods of utilizing phosphoketolase enzymes to enhance the production yields of mevalonate, isoprenoid precursors, isoprene and/or isoprenoids are detailed in U.S. Appl. Pub. No. 2013/0089906, which is hereby incorporated by reference in its entirety.

### Promoters

Suitable promoters are used to express any of the heterologous nucleic acids or other heterologous polypeptides described herein. Suitable promoters may be used to drive isoprene synthase polypeptide to reduce degradation of isoprene synthase in the anaerobic cells.

Suitable promoters may be used to optimize the expression of isoprene synthase or and one or more MVA pathway polypeptides and/or one or more DXP pathway polypeptides in anaerobes. Any of the nucleic acids described herein (e.g., a nucleic acid encoding isoprene synthase polypeptide, one or more MVA pathway polypeptides, or one or more DXP pathway polypeptides) may be operably linked to a promoter. Any of the promoters described herein may be used, such as promoter(s) from A. Woodii, including but not limited to the promoters Awo1181 and/or Awo1194, and promoters from Clostridium such as the pFdx promoter of C. sporogenes, which is described in the Examples of the present disclosure.

High expression levels in certain anaerobic cells may cause degradation of engineered polypeptide(s) including isoprene synthase. To improve isoprene production, an inducible expression system that allows both the timing and magnitude of expression of engineered polypeptide(s) to be controlled may be used. The tighter control may facilitate the expression of engineered polypeptide(s) at a concentration and period during the growth of the cells that is toxic to the cells, and results in the production of higher amounts of product such as isoprene.

A promoter used in any of the cells described herein may be an inducible promoter. A gluconate-inducible expression system may be used, for example, a gluconate-inducible expression system endogenous to C. ljungdahlii. ORFs clju19880 and clju30510 are predicted to code for transcription factors that repress the expression of genes involved in gluconate import and metabolism. In the presence of gluconate, gluconate binds to and represses these transcription factors, thus allowing expression of genes involved in gluconate import and metabolism. ORF clju11610 has been annotated as “gluconokinase” in the C. ljungdahlii genome. In Corynebacterium glutamicum, the gluconate kinase (alternate name for gluconokinase) promoter exhibits the strongest increase in expression in response to gluconate induction (Frunzke et al. 2008, Mol. Microbiol., 67(2):305-22). Thus, in some aspects, the promoter is gluconate-inducible promoter. In some aspects, the promoter is from C. acetobutylicum, C. ljungdahlii, C. aceticum, or A. woodi. In some aspects, the promoter is the promoter present in clju19880 ORF, clju 11610 ORF, or clju30510 ORF in an anaerobic cell (e.g., C. ljungdahlii). In some aspects, the promoter is a promoter present in gntR1. In some aspects, the promoter is a promoter present in gntR2. In some aspects, the promoter is gluconate kinase promoter. The promoter may also be a promoter that is induced when the cells are cultured in the presence of synthesis gas.

A promoter used in any of the cells described herein may be a constitutive promoter. Constitutive promoters do not require induction by artificial means (such as IPTG for the induction of the lac operon) and hence can result in considerable cost reduction for large scale fermentations. Constitutive promoters that function in anaerobes (e.g., C. acetobutylicum, C. aceticum and C. ljungdahlii) may be used. Promoters that have low expression may be desirable in certain embodiments. The ptb (phosphotransbutyrylase) promoter of C. acetobutylicum is strongly active during the exponential growth phase of C. acetobutylicum cultures. Promoters that may be used in the present invention may have less activity than the ptb (phosphotransbutyrylase) promoter. The spoIIE (Stage II sporulation protein E) promoter, also from C. acetobutylicum, has been shown to be transiently active in mid-stationary phase. The spoIIE (Stage II sporulation protein E) promoter may be used in the present invention. Thus, in some aspects, the promoter is spoIIE promoter (e.g., Clostridium acetobutylicum spoIIE promoter). In some aspects, the promoter has a strength that is at a level lower than ptb (e.g., the promoter has a reduced ability of driving expression compared to ptb such as Clostridium acetobutylicum ptb). In some aspects, the promoter has a strength that is at a level similar to spoIIE (e.g., the promoter has a similar ability of driving expression compared to spoIIE). In some aspects, the promoter is active post-exponential growth phase. In some aspects, the promoter is active during linear growth phase. In some aspects, the promoter is active in stationary phase. In some aspects, the promoter used in any of the cells described herein is only active in the presence of syngas. In some aspects, the promoter expresses the isoprene synthase at a low level. In some aspects, the promoter expresses the isoprene synthase at a level such that the isoprene synthase does not get cleaved by a protease or a lower percentage of the isoprene synthase gets cleaved by a protease. In some aspects, the promoter derives low level expression.

Any one of the promoters characterized or used in the Examples of the present disclosure may be used.

### Vectors

Suitable vectors may be used. For example, suitable vectors may be used to optimize the expression of isoprene synthase, and one or more MVA pathway polypeptides, and/or one or more DXP pathway polypeptides in anaerobes. In some aspects, the vector contains a selective marker. Examples of selectable markers include, but are not limited to, antibiotic resistance nucleic acids (e.g., kanamycin, ampicillin, carbenicillin, gentamicin, hygromycin, phleomycin, bleomycin, neomycin, or chloramphenicol) and/or nucleic acids that confer a metabolic advantage, such as a nutritional advantage on the host cell. In some aspects, an isoprene synthase, MVA pathway nucleic acid(s), and/or DXP pathway nucleic acid(s) integrate into the genome of cells without a selective marker.

In some aspects, the vector is a shuttle vector, which is capable of propagating in two or more different host species. Exemplary shuttle vectors are able to replicate in E. coli and/or Bacillus subtilis and in an obligate anaerobe, such as Clostridium. Upon insertion of an isoprene synthase, MVA pathway nucleic acid(s) (e.g., one or more nucleic acids encoding acetyl-CoA acetyltransferase, one or more nucleic acids encoding 3-hydroxy-3-methylglutaryl-CoA (HMG-CoA) reductase, one or more nucleic acids encoding HMG-CoA synthase, one or more nucleic acids encoding MVK, one or more nucleic acids encoding PMK, one or more nucleic acids encoding MVD, and/or one or more nucleic acids encoding IDI), and/or DXP pathway nucleic acid(s) (e.g., one or more nucleic acids encoding DXS) into the shuttle vector, the shuttle vector can be introduced into an E. coli host cell for amplification and selection of the vector. Such shuttle vector (e.g., a shuttle vector comprising one or more nucleic acids encoding an isoprene synthase polypeptide, MVA pathway nucleic acid(s) (one or more nucleic acids encoding acetyl-CoA acetyltransferase, one or more nucleic acids encoding 3-hydroxy-3-methylglutaryl-CoA (HMG-CoA) reductase, one or more nucleic acids encoding HMG-CoA synthase, one or more nucleic acids encoding MVK, one or more nucleic acids encoding PMK, one or more nucleic acids encoding MVD, and/or one or more nucleic acids encoding IDI), and/or DXP pathway nucleic acid(s) (e.g., one or more nucleic acids encoding DXS)) may also be introduced into a host cell comprising methyltransferase (e.g., an E. coli host cell expressing a methyltransferase) for the purpose of obtaining methylated vector. The vector can then be isolated and introduced into an obligate anaerobic cell for expression of the isoprene synthase, MVA pathway polypeptide, or a DXP pathway polypeptide. Any suitable shuttle vector or plasmid may be used, such as any of the shuttle plasmids described in the present disclosure or shuttle plasmids described in Heap et al. (Journal of Microbiological Methods 78 (2009) 79-85).

In some aspects, any of the cells described herein are introduced with one vector (e.g., shuttle plasmid DNA) harboring one or more nucleic acids encoding an isoprene synthase polypeptide, MVA pathway nucleic acid(s) (one or more nucleic acids encoding acetyl-CoA acetyltransferase, one or more nucleic acids encoding 3-hydroxy-3-methylglutaryl-CoA (HMG-CoA) reductase, one or more nucleic acids encoding HMG-CoA synthase, one or more nucleic acids encoding MVK, one or more nucleic acids encoding PMK, one or more nucleic acids encoding MVD, and/or one or more nucleic acids encoding IDI), and/or DXP pathway nucleic acid(s) (e.g., one or more nucleic acids encoding DXS). A dual plasmid system may also be used. Two different plasmids carrying one or more of the above-mentioned nucleic acids may be used. Each of the two different plasmids carries a different selection marker. In some aspects, the plasmid(s) are stably transformed in anaerobic cells.

Any one of the vectors characterized or used in the Examples of the present disclosure may be used.

Source Organisms

Isoprene synthase and/or MVA pathway nucleic acids (and their encoded polypeptides) and/or DXP pathway nucleic acids (and their encoded polypeptides) can be obtained from any organism that naturally contains isoprene synthase and/or MVA pathway nucleic acids and/or DXP pathway nucleic acids. As noted above, isoprene is formed naturally by a variety of organisms, such as bacteria, yeast, plants, and animals. Some organisms contain the MVA pathway for producing isoprene (FIG. 1). Isoprene synthase nucleic acids can be obtained, e.g., from any organism that contains an isoprene synthase. MVA pathway nucleic acids can be obtained, e.g., from any organism that contains the MVA pathway. DXP pathway nucleic acids can be obtained, e.g., from any organism that contains the DXP pathway.

Exemplary sources for isoprene synthases, MVA pathway polypeptides and/or DXP pathway polypeptides which can be used are also described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/078457, and WO2010/148256.

In some aspects, the source organism is a yeast, such as Saccharomyces sp., Schizosaccharomyces sp., Pichia sp., or Candida sp.

In some aspects, the source organism is a bacterium, such as strains of Bacillus such as B. lichenformis or B. subtilis, strains of Pantoea such as P. citrea, strains of Pseudomonas such as P. alcaligenes, strains of Streptomyces such as S. lividans or S. rubiginosus, strains of Escherichia such as E. coli, strains of Enterobacter, strains of Streptococcus, or strains of Archaea such as Methanosarcina mazei.

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In some aspects, the source organism is a gram-positive bacterium. Non-limiting examples include strains of Streptomyces (e.g., S. lividans, S. coelicolor, or S. griseus) and Bacillus. In some aspects, the source organism is a gram-negative bacterium, such as E. coli or Pseudomonas sp.

In some aspects, the source organism is a plant, such as a plant from the family Fabaceae, such as the Faboideae subfamily. In some aspects, the source organism is kudzu, poplar (such as Populus alba×tremula CAC35696), aspen (such as Populus tremuloides), or Quercus robur.

In some aspects, the source organism is an algae, such as a green algae, red algae, glaucophytes, chlorarachniophytes, euglenids, chromista, or dinoflagellates.

In some aspects, the source organism is a cyanobacteria, such as cyanobacteria classified into any of the following groups based on morphology: Chroococcales, Pleurocapsales, Oscillatoriales, Nostocales, or Stigonematales.

Transformation Methods

Nucleic acids encoding isoprene synthase and/or MVA pathway polypeptides and/or DXP pathway polypeptides can be inserted into an anaerobic microorganism using suitable techniques. Transformation techniques may be used according to methods described in, e.g., Leang et al. (Appl. Environ. Microbiol (2013) 79(4):1102), “Handbook on Clostridia” (P. Durre, ed., 2004) and Current Protocols in Molecular Biology (F. M. Ausubel et al. (eds) Chapter 9, 1987; Sambrook et al., Molecular Cloning: A Laboratory Manual, 2^{nd} ed., Cold Spring Harbor, 1989; and Campbell et al., Curr. Genet. 16:53-56, 1989. For obligate anaerobic host cells, such as Clostridium, electroporation, as described by Davis et al., (“Gene Cloning in Clostridia” in Durre, P., Ed. Handbook on Clostridia. Taylor & Francis, 2005). Other transformation techniques known to one skilled in the art can also be used. The introduced nucleic acids may be integrated into chromosomal DNA or genome or maintained as extrachromosomal replicating sequences. The introduced nucleic acids may be stably expressed in the cells. The introduced nucleic acids may be on a vector or vectors.

For example, strains of anaerobes may be transformed by one or more of the methods as described in the present disclosure. The methods include, but are not limited to: (i) electroporation, whereby cells are exposed to high intensity electrical fields which cause the cell membrane to become transiently porous, thus allowing the entry of DNA into the cell; (ii) conjugal transfer (or conjugation) of plasmid DNA from a donor organism such as E. coli; (iii) protoplast transformation, whereby the cell wall from the cell is stripped away enzymatically or chemically to form protoplasts (for example, when incubated with plasmid DNA, protoplasts will take up the plasmids into their cytoplasm); and/or (iv) Gene Gun (biolistic particle delivery system), whereby a small heavy metal particle is coated with plasmid DNA and subsequently propelled at high speed towards the anaerobic cells (for example, some particles penetrate the cells, thus delivering the plasmid DNA to the cells).

Growth Conditions

The anaerobic cells of any of the compositions or methods described herein are capable of replicating and/or producing isoprene or an industrial bio-product in a fermentation system that is substantially free of oxygen. In some embodiments, the fermentation system contains a carbohydrate as the energy and/or carbon source. In some embodiments, the fermentation system contains carbohydrate and hydrogen as an energy and/or carbon source. In some aspects, the fermentation system contains syngas as the carbon and/or energy source. In some aspects, the anaerobic cells are initially grown in a medium comprising a carbon source other than syngas and then switched to syngas as the carbon source.

The compositions and methods of the invention utilize substantially oxygen-free conditions. In one aspect, substantially oxygen-free conditions are conditions under which anaerobic organisms can grow and/or produce the desired products. The conditions can refer to the fermentation system (e.g., bioreactor) in addition to the culture medium. In other aspects, substantially oxygen-free conditions refers to fermentation system wherein there is less than about any of 5, 4, 3, 2, 1, 0.5, 0.2, or 0.1% by weight of oxygen. In some aspects, the fermentation system comprises less than about 0.01% by weight of oxygen. In some aspects, the fermentation system comprises less than about 0.001% by weight of oxygen.

In some aspects, the fermentation system comprises less than about 100 ppm of oxygen. In some aspects, fermentation system comprises less than about 90, 80, 70, 60, 50, 40, 30, 20, 10, 5, 2, or 1 ppm of oxygen. In some aspects, the amount of oxygen in the fermentation system is at a level low enough that an obligate anaerobe is able to reproduce and/or produce isoprene. In some aspects, the amount of oxygen in the fermentation system is at a level low enough that a facultative anaerobe favors anaerobic fermentation over aerobic respiration.

In some aspects, steps are taken to remove oxygen from the syngas or other culture medium. Oxygen can be removed by adding a catalyst and optionally adding hydrogen to the culture medium or syngas. In some aspects, the catalyst is copper.

Anaerobic cells may adapt to growth in various conditions and/or adapt to change of conditions (such as change to growth on syngas). For example, anaerobic cells may be adapted (e.g., rapidly adapted) for change of conditions (e.g., growth media) such as from heterotrophic growth on fructose-containing media to autotrophic growth on fructose-free media supplemented with syngas. In some aspects, cells such as Clostridium ljungdahlii may be adapted to change from one media to another media using methods described in examples herein. The transformation methods and growth conditions may be any of those described herein including those described in the Examples of the present disclosure.

### Transformation of Methylated or Unmethylated DNA

The cells described herein may be transformed with methylated DNA (e.g., methylated shuttle plasmid DNA) or unmethylated DNA (e.g., unmethylated shuttle plasmid DNA). In some aspects, the heterologous nucleic acids (e.g., a shuttle vector comprising one or more heterologous nucleic acids) have not been methylated when introduced to the cells. In some aspects, the heterologous nucleic acids (e.g., a shuttle vector comprising one or more heterologous nucleic acids) have been methylated when introduced to the cells. In some aspects, the shuttle plasmid DNA (e.g., methylated shuttle plasmid DNA) comprises one or more nucleic acids encoding an isoprene synthase polypeptide, MVA pathway nucleic acid(s) (one or more nucleic acids encoding acetyl-CoA acetyltransferase, one or more nucleic acids encoding 3-hydroxy-3-methylglutaryl-CoA (HMG-CoA) reductase, one or more nucleic acids encoding HMG-CoA synthase, one or more nucleic acids encoding MVK, one or more nucleic acids encoding PMK, one or more nucleic acids encoding MVD, and/or one or more nucleic acids encoding IDI), and/or DXP pathway nucleic acid(s) (e.g., one or more nucleic acids encoding DXS).

In some aspects, the cells are transformed with methylated DNA. DNA may be methylated by in vitro or in vivo methods known to one skilled in the art. For example, a DNA such as a plasmid DNA (e.g., shuttle plasmid DNA) may be methylated prior to transformation into anaerobes such as obligate anaerobes (e.g., clostridium) or acetobacteria strains to protect the plasmid DNA from degradation by restriction endonucleases in the host cells. Methylation can be performed in vivo, by transforming shuttle plasmids into a strain (e.g., E. coli) expressing a methyltransferase (e.g., a methyltransferase from Bacillus subtilis phage Φ3T). After isolation of the methylated DNA, the methylated DNA (e.g., shuttle plasmids) may be transformed into host anaerobic cells (e.g., A. woodii, C. aceticum, or C. ljungdahlii). Methods of DNA methylation are also provided as follows. DNA may be methylated in vivo in strains of E. coli expressing endogenous methyltransferases but not expressing a heterologous methyltransferase. DNA may be methylated in vivo in strains of E. coli expressing endogenous methyltransferases and also expressing a heterologous methyltransferase. DNA may also be methylated in vitro, using one or more purified methyltransferase enzymes available for purchase from commercial vendors (e.g. New England Biolabs).

In some aspects, the cells are transformed with unmethylated DNA (e.g., unmethylated plasmid DNA such as unmethylated shuttle vector DNA). The transformed unmethylated DNA (e.g., shuttle vector DNA) may not be modified and/or degraded by the restriction and modification (“RM”) system in the cells. See Dong H et al., PLoS ONE 2010, 5(2):e9038. In some aspects, the cells are deficient in at least one gene in restriction and modification (“RM”) system. In some aspects, the cells are deficient in a restriction endonuclease. In some aspects, the cells are deficient in a DNA methyltransferase. In some aspects, the cells express the isoprene synthase polypeptide at a detectable level from the transformed unmethylated DNA. In some aspects, the cells can be transformed with unmethylated DNA at an efficiency similar to that with methylated DNA. In some aspects, the cells are capable of expressing the isoprene synthase polypeptide from unmethylated DNA at an efficiency similar to that from methylated DNA.

Carbohydrates as a Carbon Source and/or Energy Source

Any of the cells described herein are capable of using carbohydrates as a source of energy and/or carbon. Carbohydrates are compounds that consist only of carbon, hydrogen, and oxygen atoms, in any ratio. In some embodiments, the carbohydrate comprises fructose. In some embodiments, the carbohydrate comprises glucose. In some embodiments, the carbohydrate can be used as carbon source for cells (e.g., for producing mevalonate, isoprene, or other industrial bio-product). In some embodiments, the carbohydrate can be used as energy source for cells (e.g., for producing mevalonate, isoprene, or other industrial bio-product).

In some embodiments, the carbohydrate (e.g., glucose or fructose) comprises about any of 100%, 99%, 98% 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the carbon source and/or energy source by weight. In some embodiments, the carbohydrate (e.g., glucose or fructose) comprises about any of 100%, 99%, 98% 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the carbon source and/or energy source by volume.

In some embodiments, the carbon and/or energy source comprises at least about 0.1% to about 40% carbohydrate (e.g., glucose or fructose). In some embodiments, the carbon and/or energy source comprises at least about 1% to about 30% carbohydrate. In some embodiments, the carbon and/or energy source comprises at least about 5% to about 27% carbohydrate. In some embodiments, the carbon and/or energy source comprises at least about 6% to about 26% carbohydrate. In some embodiments, the carbon and/or energy source comprises about 6% carbohydrate (e.g., fructose). In some embodiments, the carbon and/or energy source comprises about 26% carbohydrate (e.g., glucose).

Carbohydrates Combined with Hydrogen (H₂) and Carbon Dioxide (CO₂) as a Carbon Source and/or Energy Source

Any of the cells described herein are capable of using carbohydrates combined with hydrogen (H₂) and carbon dioxide (CO₂) as a source of energy and/or carbon. In some embodiments, the carbohydrate comprising fructose is combined with 4% H₂ and 5% CO₂. In some embodiments, the carbohydrate comprising glucose is combined with 4% H₂ and 5% CO₂. In some embodiments, the carbohydrate combined with H₂ and CO₂ can be used as carbon source for cells (e.g., for producing mevalonate, isoprene, or other industrial bio-product). In some embodiments, the carbohydrate combined with H₂ and CO₂ can be used as energy source for cells (e.g., for producing mevalonate, isoprene, or other industrial bio-product).

In some embodiments, the carbohydrate (e.g., glucose or fructose) comprises about any of 100%, 99%, 98% 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the carbon source and/or energy source by weight. In some embodiments, the carbohydrate (e.g., glucose or fructose) comprises about any of 100%, 99%, 98% 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the carbon source and/or energy source by volume.

The carbohydrate may be combined with any proportion of H₂ and CO₂. In some embodiments, the H₂ comprises about any of 100%, 99%, 98% 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the carbon source and/or energy source by volume. In some embodiments, the CO₂ comprises about any of 100%, 99%, 98% 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% of the carbon source and/or energy source by volume.

In some embodiments, the carbon and/or energy source comprises at least about 0.1% to about 10% H₂, at least about 0.1% to about 10% CO₂, and at least about 0.1% to about 40% carbohydrate (e.g., glucose or fructose). In some embodiments, the carbon and/or energy source comprises at least about 1% to about 8% H₂, at least about 1% to about 8% CO₂, and at least about 1% to about 30% carbohydrate. In some embodiments, the carbon and/or energy source comprises at least about 3% to about 6% H₂, at least about 3% to about 6% CO₂, and at least about 5% to about 27% carbohydrate. In some embodiments, the carbon and/or energy source comprises at least about 4% to about 5% H₂, at least about 4% to about 5% CO₂, and at least about 6% to about 26% carbohydrate. In some embodiments, the carbon and/or energy source comprises about 4% H₂, about 5% CO₂, and about 6% carbohydrate (e.g., fructose). In some preferred embodiments, the carbon and/or energy source comprises about 4% H₂, about 5% CO₂, and about 26% carbohydrate (e.g., glucose).

In some embodiments, the production of an industrial bio-product (e.g., mevalonate) from a combination of carbohydrate, H₂, and CO₂ is increased by at least about 1.1×, 2×, 3×, 4×, 5×, 6×, 7×, 8×, 9×, 10×, 11×, 12×, 13×, 14×, 15×, 16×, 17×, 18×, 19×, 20×, 25×, 30×, 35×, 40×, 45×, 50×, 55×, 60×, 65×, 70×, 75×, 80×, 85×, 90×, 95×, or 100×. In some embodiments, the production of an industrial bio-product (e.g., mevalonate) from a combination of carbohydrate, H₂, and CO₂ is increased by at least about 105×, 110×, 115×, 120×, 125×, 130×, 140×, 145×, 150×, 155×, 160×, 165×, 170×, 175×, 180×, 185×, 190×, 195×, 200×, 220×, 240×, 260×, 280×, 300×, 320×, 340×, 360×, 380×, 400×, 500×, 600×, 700×, 800×, or 1000×. In some embodiments, the production of an industrial bio-product (e.g., mevalonate) from a combination of carbohydrate, H₂, and CO₂ is increased by at least about 1.1× to about 200× as compared to the amount of product produced using a carbon source and/or energy source that does not comprise a combination of carbohydrate, H₂ and CO₂ (e.g., carbohydrate alone or syngas alone). In some embodiments, the production of an industrial bio-product (e.g., mevalonate) from a combination of carbohydrate, H₂, and CO₂ is increased by at least about 10× to about 180× as compared to the amount of product produced using a carbon source and/or energy source that does not comprise a combination of carbohydrate, H₂ and CO₂ (e.g., carbohydrate alone or syngas alone). In some embodiments, the production of an industrial bio-product (e.g., mevalonate) from a combination of carbohydrate, H₂, and CO₂ is increased by at least about 20× to about 160× as compared to the amount of product produced using a carbon source and/or energy source that does not comprise a combination of carbohydrate, H₂ and CO₂ (e.g., carbohydrate alone or syngas alone). In some embodiments, the production of an industrial bio-product (e.g., mevalonate) from a combination of carbohydrate, H₂, and CO₂ is increased by at least about 40× to about 140× as compared to the amount of product produced using a carbon source and/or energy source that does not comprise a combination of carbohydrate, H₂ and CO₂ (e.g., carbohydrate alone or syngas alone). In some embodiments, the production of an industrial bio-product (e.g., mevalonate) from a combination of carbohydrate, H₂, and CO₂ is increased by at least about 60× to about 120× as compared to the amount of product produced using a carbon source and/or energy source that does not comprise a combination of carbohydrate, H₂ and CO₂ (e.g., carbohydrate alone or syngas alone). In some embodiments, the production of an industrial bio-product (e.g., mevalonate) from a combination of carbohydrate, H₂, and CO₂ is increased by at least about 80× to about 100× as compared to the amount of product produced using a carbon source and/or energy source that does not comprise a combination of carbohydrate, H₂ and CO₂ (e.g., carbohydrate alone or syngas alone). In some embodiments, the production of an industrial bio-product (e.g., mevalonate) from a combination of carbohydrate, H₂, and CO₂ is increased by at least about 100× as compared to the amount of product produced using a carbon source and/or energy source that does not comprise a combination of carbohydrate, H₂ and CO₂ (e.g., carbohydrate alone or syngas alone). In some embodiments, the production of an industrial bio-product (e.g., mevalonate) from a combination of carbohydrate, H₂, and CO₂ is increased by at least about 125× as compared to the amount of product produced using a carbon source and/or energy source that does not comprise a combination of carbohydrate, H₂ and CO₂ (e.g., carbohydrate alone or syngas alone). In some embodiments, the production of an industrial bio-product (e.g., mevalonate) from a combination of carbohydrate, H₂, and CO₂ is increased by at least about 150× as compared to the amount of product produced using a carbon source and/or energy source that does not comprise a combination of carbohydrate, H₂ and CO₂ (e.g., carbohydrate alone or syngas alone). In some embodiments, the production of an industrial bio-product (e.g., mevalonate) from a combination of carbohydrate, H₂, and CO₂ is increased by at least about 200× as compared to the amount of product produced using a carbon source and/or energy source that does not comprise a combination of carbohydrate, H₂ and CO₂ (e.g., carbohydrate alone or syngas alone).

### Syngas

Any of the cells described herein are capable of using syngas as a source of energy and/or carbon. Syngas comprises CO and H₂. In some aspects, the syngas comprises CO, CO₂, and H₂. In some aspects, the syngas further comprises H₂O and/or N₂. For example, the syngas may comprise CO, H₂, and H₂O (e.g., CO, H₂, H₂O and N₂). The syngas may comprise CO, H₂, and N₂. The syngas may comprise CO, CO₂, H₂, and H₂O (e.g., CO, CO₂, H₂, H₂O and N₂). The syngas may comprise CO, CO₂, H₂, and N₂. The N₂ may be replaced with Ar as in examples herein. The CO and/or CO₂ in the synthesis gas may be used as carbon source for cells (e.g., for producing isoprene). The H₂ in the synthesis gas may be used as energy source for cells (e.g., for producing isoprene).

In some aspects, the molar ratio of hydrogen to carbon monoxide in the syngas is about any of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0, 5.0, or 10.0. In some aspects, the syngas comprises about any of 10, 20, 30, 40, 50, 60, 70, 80, or 90% by volume carbon monoxide. In some aspects, the syngas comprises about any of 10, 20, 30, 40, 50, 60, 70, 80, or 90% by volume hydrogen. In some aspects, the syngas comprises about any of 10, 20, 30, 40, 50, 60, 70, 80, or 90% by volume carbon dioxide. In some aspects, the syngas comprises about any of 10, 20, 30, 40, 50, 60, 70, 80, or 90% by volume water. In some aspects, the syngas comprises about any of 10, 20, 30, 40, 50, 60, 70, 80, or 90% by volume nitrogen.

The synthesis gas of the present invention may be derived from natural or synthetic sources. The source from which the syngas is derived is referred to as a “feedstock.” In some aspects, the syngas is derived from biomass (e.g., wood, switch grass, agriculture waste, municipal waste) or carbohydrates (e.g., sugars). In other aspects, the syngas is derived from coal, petroleum, kerogen, tar sands, oil shale, natural gas, or a mixture thereof. In other aspects, the syngas is derived from rubber, such as from rubber tires. In some aspects, the syngas is derived from a mixture (e.g., blend) of biomass and coal. In some aspects, the mixture has about or at least about any of 1%, 2%, 5%, 10%, 15%, 20%, 25%, 30%, 35%, 40%, 45%, 50%, 55%, 60%, 65%, 70%, 75%, 80%, 90%, 95%, or 99% biomass. In some aspects, the mixture has about or at least about any of 1%, 2%, 5%, 10%, 15%, 20%, 25%, 30%, 35%, 40%, 45%, 50%, 55%, 60%, 65%, 70%, 75%, 80%, 90%, 95%, or 99% coal. In some aspects, the ratio of biomass to coal in the mixture is about any of 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, or 95:5.

Syngas can be derived from a feedstock by a variety of processes, including methane reforming, coal liquefaction, co-firing, fermentative reactions, enzymatic reactions, and biomass gasification. Biomass gasification is accomplished by subjecting biomass to partial oxidation in a reactor at temperatures above about 700° C. in the presence of less than a stoichiometric amount of oxygen. The oxygen is introduced into the bioreactor in the form of air, pure oxygen, or steam. Gasification can occur in three main steps: 1) initial heating to dry out any moisture embedded in the biomass; 2) pyrolysis, in which the biomass is heated to 300-500° C. in the absence of oxidizing agents to yield gas, tars, oils and solid char residue; and 3) gasification of solid char, tars and gas to yield the primary components of syngas. Co-firing is accomplished by gasification of a coal/biomass mixture. The composition of the syngas, such as the identity and molar ratios of the components of the syngas, can vary depending on the feedstock from which it is derived and the method by which the feedstock is converted to syngas.

Synthesis gas can contain impurities, the nature and amount of which vary according to both the feedstock and the process used in production. Fermentations may be tolerant to some impurities, but there remains the need to remove from the syngas materials such as tars and particulates that might foul the fermentor and associated equipment. It is also advisable to remove compounds that might contaminate the isoprene product such as volatile organic compounds, acid gases, methane, benzene, toluene, ethylbenzene, xylenes, H₂S, COS, CS₂, HCl, O₃, organosulfur compounds, ammonia, nitrogen oxides, nitrogen-containing organic compounds, and heavy metal vapors. Removal of impurities from syngas can be achieved by one of several means, including gas scrubbing, treatment with solid-phase adsorbents, and purification using gas-permeable membranes.

Examples of other fermentation systems and culture conditions which can be use are described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/078457, and WO2010/148256.

In some aspects, the culture medium is prepared using anoxic techniques. In some aspects, the culture medium comprises one or more of NH₄Cl, NaCl, KCl, KH₂PO₄, MgSO₄.7H₂O, CaCl₂.2H₂O, NaHCO₃, yeast extract, cysteine hydrochloride, Na₂S.9H₂O, trace metals, and vitamins. In some aspects, the culture medium contains, per liter, about 1.0 g NH₄Cl, about 0.8 g NaCl, about 0.1 g KCl, about 0.1 g KH₂PO₄, about 0.2 g MgSO₄.7H₂O, about 0.02 g CaCl₂.2H₂O, about 1.0 g NaHCO₃, about 1.0 g yeast extract, about 0.2 g cysteine hydrochloride, about 0.2 g Na₂S.9H₂O, about 10 mL trace metal solution, and about 10 mL vitamin solution. In some aspects, the culture condition comprises mevalonate. The culture condition and culture medium may be according to any of conditions and medium described in the Examples of the present disclosure.

### Bioreactors

A variety of different types of reactors can be used for production of isoprene or other industrial bio-products. In some embodiments, a carbohydrate is used as energy and/or carbon source. In some embodiments, a carbohydrate and hydrogen are used as energy and/or carbon source. In some embodiments, synthesis gas is used as energy and/or carbon source. There are a large number of different types of fermentation processes that are used commercially. Bioreactors for use in the present invention should be amenable to anaerobic conditions. The bioreactor can be designed to optimize the retention time of the cells, the residence time of liquid, and the sparging rate of syngas.

In various aspects, the cells are grown using any known mode of fermentation, such as batch, fed-batch, continuous, or continuous with recycle processes. In some aspects, a batch method of fermentation is used. Classical batch fermentation is a closed system where the composition of the media is set at the beginning of the fermentation and is not subject to artificial alterations during the fermentation. Thus, at the beginning of the fermentation the cell medium is inoculated with the desired host cells and fermentation is permitted to occur adding nothing to the system. Typically, however, “batch” fermentation is batch with respect to the addition of carbon source and attempts are often made at controlling factors such as pH and oxygen concentration. In batch systems, the metabolite and biomass compositions of the system change constantly until the time the fermentation is stopped. Within batch cultures, cells moderate through a static lag phase to a high growth log phase and finally to a stationary phase where growth rate is diminished or halted. In some aspects, cells in log phase are responsible for the bulk of the isoprene production. In some aspects, cells in stationary phase produce isoprene.

In some aspects, a variation on the standard batch system is used, such as the Fed-Batch system. Fed-Batch fermentation processes comprise a typical batch system with the exception that the carbon source (e.g. syngas, glucose, fructose) is added in increments as the fermentation progresses. Fed-Batch systems are useful when catabolite repression is apt to inhibit the metabolism of the cells and where it is desirable to have limited amounts of carbon source in the cell medium. Fed-batch fermentations may be performed with the carbon source (e.g., syngas, glucose, fructose) in a limited or excess amount. Measurement of the actual carbon source concentration in Fed-Batch systems is difficult and is therefore estimated on the basis of the changes of measurable factors such as pH, dissolved oxygen, and the partial pressure of waste gases such as CO₂. Batch and Fed-Batch fermentations are common and well known in the art and examples may be found in Brock, Biotechnology: A Textbook of Industrial Microbiology, Second Edition (1989) Sinauer Associates, Inc.

In some aspects, continuous fermentation methods are used. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth.

Continuous fermentation allows for the modulation of one factor or any number of factors that affect cell growth or isoprene production. For example, one method maintains a limiting nutrient such as the carbon source or nitrogen level at a fixed rate and allows all other parameters to moderate. In other systems, a number of factors affecting growth can be altered continuously while the cell concentration (e.g., the concentration measured by media turbidity) is kept constant. Continuous systems strive to maintain steady state growth conditions. Thus, the cell loss due to media being drawn off is balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, Biotechnology: A Textbook of Industrial Microbiology, Second Edition (1989) Sinauer Associates, Inc., which is hereby incorporated by reference in its entirety, particularly with respect to cell culture and fermentation conditions.

A variation of the continuous fermentation method is the continuous with recycle method. This system is similar to the continuous bioreactor, with the difference being that cells removed with the liquid content are returned to the bioreactor by means of a cellmass separation device. Cross-filtration units, centrifuges, settling tanks, wood chips, hydrogels, and/or hollow fibers are used for cellmass separation or retention. This process is typically used to increase the productivity of the continuous bioreactor system, and may be particularly useful for anaerobes, which may grow more slowly and in lower concentrations than aerobes.

In one aspect, a membrane bioreactor can be used for the growth and/or fermentation of the anaerobic cells described herein, in particular, if the cells are expected to grow slowly. A membrane filter, such as a crossflow filter or a tangential flow filter, can be operated jointly with a liquid fermentation bioreactor that produces isoprene gas. Such a membrane bioreactor can enhance fermentative production of isoprene gas by combining fermentation with recycling of select broth components that would otherwise be discarded. The MBR filters fermentation broth and returns the non-permeating component (filter “retentate”) to the reactor, effectively increasing reactor concentration of cells, cell debris, and other broth solids, while maintaining specific productivity of the cells. This substantially improves titer, total production, and volumetric productivity of isoprene, leading to lower capital and operating costs.

The liquid filtrate (or permeate) is not returned to the reactor and thus provides a beneficial reduction in reactor volume, similar to collecting a broth draw-off. However, unlike a broth draw-off, the collected permeate is a clarified liquid that can be easily sterilized by filtration after storage in an ordinary vessel. Thus, the permeate can be readily reused as a nutrient and/or water recycle source. A permeate, which contains soluble spent medium, may be added to the same or another fermentation to enhance isoprene production.

Isoprene Production

Also provided herein are methods of producing isoprene comprising culturing anaerobic cells (e.g., obligate anaerobic cells or facultative anaerobic cells) comprising one or more heterologous nucleic acids encoding isoprene synthase polypeptide in a substantially oxygen-free culture condition under suitable conditions for the production of isoprene. In some embodiments, a carbohydrate is used as energy and/or carbon source. In some embodiments, a carbohydrate and hydrogen are used as energy and/or carbon source. In some embodiments, synthesis gas is used as energy and/or carbon source. In some embodiments, the synthesis gas (and/or carbohydrate and hydrogen) are used as energy and/or carbon source. Syngas may be a source of hydrogen. The isoprene is produced from any of the cells described herein and according to any of the methods described herein. Also provided herein are isoprene compositions produced by any of the methods provided herein.

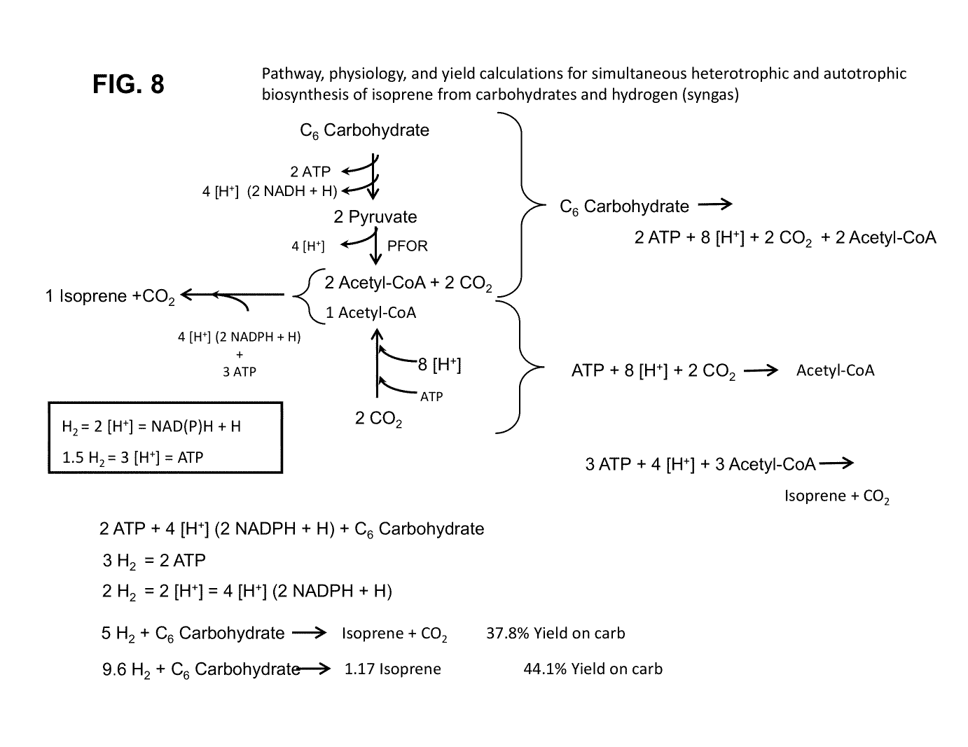

Any of the anaerobic cells may be used for the purpose of producing isoprene from carbohydrates. In other embodiments, the anaerobic cells may be used for the purpose of making isoprene from carbohydrate and hydrogen. In still other embodiments, the anaerobic cells may be used for production of isoprene from syngas. The strains described herein that are engineered to produce isoprene from syngas may be used to convert carbohydrates to isoprene with supplementation by hydrogen (or syngas) to increase the efficiency and yield of isoprene formation from carbohydrates. Simultaneous operation of autotrophic metabolism decreases the carbon footprint of the isoprene biosynthesis by allowing the capture of CO₂ that would have otherwise been released as an off-gas. This capture increases the efficiency and utilization of the carbon from biomass yielding more isoprene product per gram biomass consumed. The simultaneous utilization of carbohydrates and hydrogen is illustrated in FIG. 8. The calculations shown demonstrate how reducing power provided by hydrogen can supplement what can be metabolically derived from carbohydrates alone with respect to conservation of carbon. The use of C6 carbohydrates shown in FIG. 8 are for the purpose of demonstrating the yield calculations, other carbohydrates can also be metabolized by this process. Thus, also provided herein are anaerobic cells comprising one or more heterologous nucleic acids encoding isoprene synthase polypeptide in a culture condition (e.g., a substantially oxygen-free culture) comprising carbohydrate(s) and hydrogen. Also provided herein are methods of producing isoprene comprising culturing anaerobic cells comprising one or more heterologous nucleic acids encoding isoprene synthase polypeptide in a suitable condition (e.g., substantially oxygen-free culture condition) for producing isoprene, wherein the culture condition comprises carbohydrate(s) and hydrogen. The carbohydrate(s) may be used as carbon source and/or energy source for producing isoprene. Hydrogen may be used as energy source. In some aspects, the cells further comprise one or more nucleic acid encoding MVA pathway polypeptide(s) described herein (e.g., acetyl-CoA acetyltransferase, 3-hydroxy-3-methylglutaryl-CoA (HMG-CoA) reductase, HMG-CoA synthase, MVK, PMK, MVD, and/or IDI), and/or one or more nucleic acid encoding DXP pathway polypeptide(s) (e.g., DXS) described herein. In some aspects, the anaerobic cells may be any of the cells described herein. Any of the isoprene synthases or variants thereof described herein, any of the anaerobic strains described herein, any of the promoters described herein, and/or any of the vectors described herein may be used to produce isoprene using carbohydrate(s) and hydrogen.

In some aspects of any of the methods provided herein, isoprene synthase polypeptide is less susceptible to degradation (e.g., degradation by protease(s)) in the cells during culturing.

In some aspects, the isoprene synthase polypeptide is less susceptible to degradation in the cells when using inducible promoter or constitutive promoter (e.g., low expression constitutive promoter) for driving the expression of isoprene synthase polypeptide. In some aspects, the degradation of isoprene synthase polypeptide in the cells when using the inducible promoter or constitutive promoter (e.g., low expression constitutive promoter) is less compared to the degradation when using a constitutive promoter and/or high expression promoter (e.g., high expression constitutive promoter) for driving expression of the isoprene synthase polypeptide.

In some aspects, the isoprene synthase polypeptide is less susceptible to degradation in the cells when using the host anaerobic cells (e.g., cells that are deficient in protease(s)) in which the isoprene synthase polypeptide is not degraded or more resistant to degradation by protease(s). In some aspects, the degradation of isoprene synthase polypeptide in the cells when using such host anaerobic cells is less compared to the degradation of isoprene synthase polypeptide in the cells when not using such host anaerobic cells.

In some aspects, the isoprene synthase polypeptide is less susceptible to degradation in the cells when using isoprene synthase polypeptide (e.g., a variant) having more resistance to degradation by protease(s) in the cells. In some aspects, the isoprene synthase polypeptide (e.g., a variant) has mutation(s) in the wild-type or naturally occurring isoprene synthase, and wherein the isoprene synthase polypeptide having mutation(s) is more resistant to degradation by protease(s). In some aspects, the degradation of isoprene synthase polypeptide in the cells when using such isoprene synthase polypeptide is less compared to the degradation of isoprene synthase polypeptide in the cells when not using such isoprene synthase polypeptide. In some aspects, the degradation of isoprene synthase polypeptide in the cells when using such isoprene synthase polypeptide is less compared to the degradation of isoprene synthase polypeptide in the cells when using a wild-type or naturally occurring isoprene synthase.

In some aspects, the isoprene synthase polypeptide is less susceptible to degradation in the cells when using (a) inducible promoter or constitutive promoter (e.g., low expression constitutive promoter) for driving the expression of isoprene synthase polypeptide, (b) using the host anaerobic cells (e.g., cells that are deficient in protease(s)) in which the isoprene synthase polypeptide is not degraded or more resistant to degradation by protease(s), and/or (c) using isoprene synthase polypeptide (e.g., a variant) having more resistance to degradation by protease(s) in the cells. In some aspects, the degradation when using (a), (b), and/or (c) is less compared to the degradation when not using (a), (b), and/or (c).

In some aspects of the invention, any of the anaerobic cells described herein are cultured in a fermentation system using syngas under conditions permitting the production of isoprene by the cells. In some aspects, the amount of isoprene produced is measured at the peak absolute productivity time point. In some aspects, the peak absolute productivity for the cells is about any of the amounts of isoprene disclosed herein. In some aspects, the amount of isoprene produced is measured at the peak specific productivity time point. In some aspects, the peak specific productivity for the cells is about any of the amounts of isoprene per cell disclosed herein. In some aspects, the cumulative, total amount of isoprene produced is measured. In some aspects, the cumulative total productivity for the cells is about any of the amounts of isoprene disclosed herein.

In some aspects, any of the cells described herein (for examples the cells in culture) produce isoprene at greater than about any of or about any of 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, or more nmole of isoprene/gram of cells for the wet weight of the cells/hour (nmole/g_{wcm}/hr). In some aspects, the amount of isoprene is between about 2 to about 5,000 nmole/g_{wcm}/hr, such as between about 2 to about 100 nmole/g_{wcm}/hr, about 100 to about 500 nmole/g_{wcm}/hr, about 150 to about 500 nmole/g_{wcm}/hr, about 500 to about 1,000 nmole/g_{wcm}/hr, about 1,000 to about 2,000 nmole/g_{wcm}/hr, or about 2,000 to about 5,000 nmole/g_{wcm}/hr. In some aspects, the amount of isoprene is between about 20 to about 5,000 nmole/g_{wcm}/hr, about 100 to about 5,000 nmole/g_{wcm}/hr, about 200 to about 2,000 nmole/g_{wcm}/hr, about 200 to about 1,000 nmole/g_{wcm}/hr, about 300 to about 1,000 nmole/g_{wcm}/hr, or about 400 to about 1,000 nmole/g_{wcm}/hr.

In some aspects, the cells in culture produce isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 100,000, or more ng of isoprene/gram of cells for the wet weight of the cells/hr (ng/g_{wcm}/h). In some aspects, the amount of isoprene is between about 2 to about 5,000 ng/g_{wcm}/h, such as between about 2 to about 100 ng/g_{wcm}/h, about 100 to about 500 ng/g_{wcm}/h, about 500 to about 1,000 ng/g_{wcm}/h, about 1,000 to about 2,000 ng/g_{wcm}/h, or about 2,000 to about 5,000 ng/g_{wcm}/h. In some aspects, the amount of isoprene is between about 20 to about 5,000 ng/g_{wcm}/h, about 100 to about 5,000 ng/g_{wcm}/h, about 200 to about 2,000 ng/g_{wcm}/h, about 200 to about 1,000 ng/g_{wcm}/h, about 300 to about 1,000 ng/g_{wcm}/h, or about 400 to about 1,000 ng/g_{wcm}/h.

In some aspects, the cells in culture produce a cumulative titer (total amount) of isoprene at greater than about any of or about any of 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 50,000, 100,000, or more mg of isoprene/L of broth (mg/L_{broth}, wherein the volume of broth includes the volume of the cells and the cell medium). In some aspects, the amount of isoprene is between about 2 to about 5,000 mg/L_{broth}, such as between about 2 to about 100 mg/L_{broth}, about 100 to about 500 mg/L_{broth}, about 500 to about 1,000 mg/L_{broth}, about 1,000 to about 2,000 mg/L_{broth}, or about 2,000 to about 5,000 mg/L_{broth}. In some aspects, the amount of isoprene is between about 20 to about 5,000 mg/L_{broth}, about 100 to about 5,000 mg/L_{broth}, about 200 to about 2,000 mg/L_{broth}, about 200 to about 1,000 mg/L_{broth}, about 300 to about 1,000 mg/L_{broth}, or about 400 to about 1,000 mg/L_{broth}.

In some aspects, the isoprene produced by the cells in culture comprises at least about 1, 2, 5, 10, 15, 20, or 25% by volume of the fermentation offgas. In some aspects, the isoprene comprises between about 1 to about 25% by volume of the offgas, such as between about 5 to about 15%, about 15 to about 25%, about 10 to about 20%, or about 1 to about 10%.

Provided herein are anaerobic cells having enhanced isoprene production. The production of isoprene by the cells may be enhanced by the expression of one or more heterologous nucleic acids encoding the isoprene synthase polypeptide. The production of isoprene may be enhanced by about 10% to about 1,000,000 folds (e.g., about 50% to about 1,000,000 folds, about 1 to about 500,000 folds, about 1 to about 50,000 folds, about 1 to about 5,000 folds, about 1 to about 1,000 folds, about 1 to about 500 folds, about 1 to about 100 folds, about 1 to about 50 folds, about 5 to about 100,000 folds, about 5 to about 10,000 folds, about 5 to about 1,000 folds, about 5 to about 500 folds, about 5 to about 100 folds, about 10 to about 50,000 folds, about 50 to about 10,000 folds, about 100 to about 5,000 folds, about 200 to about 1,000 folds, about 50 to about 500 folds, or about 50 to about 200 folds) compared to the production of isoprene by the cells without the expression of one or more heterologous nucleic acids encoding an isoprene synthase polypeptide.

The production of isoprene by the cells according to any of the methods described herein may be enhanced (e.g., enhanced by the expression of one or more heterologous nucleic acids encoding the isoprene synthase polypeptide). The production of isoprene may be enhanced by about 10% to about 1,000,000 folds (e.g., about 50% to about 1,000,000 folds, about 1 to about 500,000 folds, about 1 to about 50,000 folds, about 1 to about 5,000 folds, about 1 to about 1,000 folds, about 1 to about 500 folds, about 1 to about 100 folds, about 1 to about 50 folds, about 5 to about 100,000 folds, about 5 to about 10,000 folds, about 5 to about 1,000 folds, about 5 to about 500 folds, about 5 to about 100 folds, about 10 to about 50,000 folds, about 50 to about 10,000 folds, about 100 to about 5,000 folds, about 200 to about 1,000 folds, about 50 to about 500 folds, or about 50 to about 200 folds) compared to the production of isoprene by the naturally-occurring cells (e.g., the cells without the expression of one or more heterologous nucleic acids encoding an isoprene synthase polypeptide). The production of isoprene may also enhanced by at least about any of 10%, 20%, 30%, 40%, 50%, 60%, 70%, 80%, 90%, 1 fold, 2 folds, 5 folds, 10 folds, 20 folds, 50 folds, 100 folds, 200 folds, 500 folds, 1000 folds, 2000 folds, 5000 folds, 10,000 folds, 20,000 folds, 50,000 folds, 100,000 folds, 200,000 folds, 500,000 folds, or 1,000,000 folds.

The isoprene can be further oligomerized to achieve fuel products or fuel compositions as exemplified in International Patent Application Publication No. WO 2010/148144. In some aspects, the system and compositions for producing a polymer of isoprene by polymerizing isoprene derived from renewable resources further comprises a catalyst for polymerizing isoprene. In some aspects, the system and compositions further comprises a polymerization initiator. The polymerization reaction can also be initiated using a vast array of different polymerization initiators or catalyst systems. The initiator or catalyst system used will be dependent upon the desired characteristics of the isoprene containing polymer being synthesized. For instance, in cases where cis-1,4-polyisoprene rubber is being made a Ziegler Natta catalyst system which is comprised of titanium tetrachloride and triethyl aluminum can be utilized. In synthesizing other types of isoprene containing polymers other types of initiator systems may be needed. For instance, isoprene containing polymers can be made using agree radical initiator, a redox initiator, an anionic initiator, or a cationic initiator. The preferred initiation or catalyst system will depend upon the polymer microstructure, molecular weight, molecular weight distribution, and chain branching desired. The preferred initiators will also depend upon whether the isoprene is being homopolymerized or copolymerized with additional monomers. In the case of copolymers the initiator used will also depend upon whether it is desirable for the polymer being made to have a random, non-random, or tapered distribution of repeat units that are derived of the particular monomers. For instance, anionic initiators or controlled free radical initiators are typically used in synthesizing block copolymers having isoprene blocks.

It is important for the initiator or catalyst system employed to be compatible with the type of polymerization system used. For instance, in emulsion polymerizations free radical initiators are typically utilized. In solution polymerizations anionic initiators, such as alkyl lithium compounds, are typically employed to initiate the polymerization. An advantage of free radical polymerization is that reactions can typically be carried out under less rigorous conditions than ionic polymerizations. Free radical initiation systems also exhibit a greater tolerance of trace impurities.

Recombinant Cells (Such as Bacterial Cells) Capable of Increased Production of Isoprenoid Precursors and/or Isoprenoids

Isoprenoids can be produced in many organisms from the synthesis of the isoprenoid precursor molecules which are the end products of the MVA pathway. As stated above, isoprenoids represent an important class of compounds and include, for example, food and feed supplements, flavor and odor compounds, and anticancer, antimalarial, antifungal, and antibacterial compounds.

As a class of molecules, isoprenoids are classified based on the number of isoprene units comprised in the compound. Monoterpenes comprise ten carbons or two isoprene units, sesquiterpenes comprise 15 carbons or three isoprene units, diterpenes comprise 20 carbons or four isoprene units, sesterterpenes comprise 25 carbons or five isoprene units, and so forth. Steroids (generally comprising about 27 carbons) are the products of cleaved or rearranged isoprenoids.

Isoprenoids can be produced from the isoprenoid precursor molecules IPP and DMAPP. These diverse compounds are derived from these rather simple universal precursors and are synthesized by groups of conserved polyprenyl pyrophosphate synthases (Hsieh et al., Plant Physiol. 2011 March; 155(3): 1079-90). The various chain lengths of these linear prenyl pyrophosphates, reflecting their distinctive physiological functions, in general are determined by the highly developed active sites of polyprenyl pyrophosphate synthases via condensation reactions of allylic substrates (dimethylallyl diphosphate (C₅-DMAPP), geranyl pyrophosphate (C₁₀-GPP), farnesyl pyrophosphate (C₁₅-FPP), geranylgeranyl pyrophosphate (C₂₀-GGPP)) with corresponding number of isopentenyl pyrophosphates (C₅-IPP) (Hsieh et al., Plant Physiol. 2011 March; 155(3):1079-90).

Production of isoprenoid precursors and/or isoprenoid can be made by using any of the recombinant host cells described here where one or more of the enzymatic pathways have been manipulated such that enzyme activity is modulated to increase carbon flow towards isoprenoid production. In addition, these cells can express one or more copies of a heterologous nucleic acid encoding an upper MVA pathway polypeptide for increased production of mevalonate, isoprene, isoprenoid precursors and/or isoprenoids. In other aspects, these cells can express one or more copies of a heterologous nucleic acid encoding an mvaE and an mvaS polypeptide (such as, but not limited to, mvaE and mvaS polypeptides from L. grayi, E. faecium, E. gallinarum, E. casseliflavus, and/or E. faecalis) for increased production of mevalonate, isoprene, isoprenoid precursors and/or isoprenoids. Any of the recombinant host cells that have been engineered for increased carbon flux to mevalonate expressing one or more copies of a heterologous nucleic acid encoding an upper MVA pathway polypeptide (e.g., an mvaE and/or an mvaS polypeptide such as, but not limited to, mvaE and mvaS polypeptides from L. grayi, E. faecium, E. gallinarum, E. casseliflavus, and/or E. faecalis) capable of increased production of mevalonate or isoprene described above can also be capable of increased production of isoprenoid precursors and/or isoprenoids. In some aspects, these cells further comprise one or more heterologous nucleic acids encoding polypeptides of the lower MVA pathway, IDI, and/or the DXP pathway, as described above, and a heterologous nucleic acid encoding a polyprenyl pyrophosphate synthase polypeptide. Without being bound to theory, it is thought that increasing the cellular production of mevalonate in cells (such as bacterial cells) by any of the compositions and methods described above will similarly result in the production of higher amounts of isoprenoid precursor molecules and/or isoprenoids. Increasing the molar yield of mevalonate production from glucose translates into higher molar yields of isoprenoid precursor molecules and/or isoprenoids, including isoprene, produced from glucose when combined with appropriate enzymatic activity levels of mevalonate kinase, phosphomevalonate kinase, diphosphomevalonate decarboxylase, isopentenyl diphosphate isomerase and other appropriate enzymes for isoprene and isoprenoid production.

As further described in greater detail in the Examples, isoprenoid precursors, such as mevalonate, can be made using different types of anaerobic microorganisms, such as C. ljungdahlii and C. acetobutylicum, using different types of carbon and/or energy sources, such as carbohydrates (e.g., fructose and glucose) in optional combination with hydrogen and/or carbon dioxide.

Types of Isoprenoids

The cells (such as bacterial cells) of the present invention that have been engineered for increased carbon flux to mevalonate are capable of increased production of isoprenoids and the isoprenoid precursor molecules mevalonate (MVA), DMAPP, and IPP. Examples of isoprenoids include, without limitation, hemiterpenoids, monoterpenoids, sesquiterpenoids, diterpenoids, sesterterpenoids, triterpenoids, tetraterpenoids, and higher polyterpenoids. In some aspects, the hemiterpenoid is prenol (i.e., 3-methyl-2-buten-1-ol), isoprenol (i.e., 3-methyl-3-buten-1-ol), 2-methyl-3-buten-2-ol, or isovaleric acid. In some aspects, the monoterpenoid can be, without limitation, geranyl pyrophosphate, eucalyptol, limonene, or pinene. In some aspects, the sesquiterpenoid is farnesyl pyrophosphate, artemisinin, or bisabolol. In some aspects, the diterpenoid can be, without limitation, geranylgeranyl pyrophosphate, retinol, retinal, phytol, taxol, forskolin, or aphidicolin. In some aspects, the triterpenoid can be, without limitation, squalene or lanosterol. The isoprenoid can also be selected from the group consisting of abietadiene, amorphadiene, carene, α-famesene, β-farnesene, farnesol, geraniol, geranylgeraniol, linalool, limonene, myrcene, nerolidol, ocimene, patchoulol, β-pinene, sabinene, γ-terpinene, terpinene and valencene.

In some aspects, the tetraterpenoid is lycopene or carotene (a carotenoid). As used herein, the term “carotenoid” refers to a group of naturally-occurring organic pigments produced in the chloroplasts and chromoplasts of plants, of some other photosynthetic organisms, such as algae, in some types of fungus, and in some bacteria. Carotenoids include the oxygen-containing xanthophylls and the non-oxygen-containing carotenes. In some aspects, the carotenoids are selected from the group consisting of xanthophylls and carotenes. In some aspects, the xanthophyll is lutein or zeaxanthin. In some aspects, the carotenoid is α-carotene, β-carotene, γ-carotene, β-cryptoxanthin or lycopene.

### Other Products

In some aspects of the invention, any of the methods described herein may be used to produce products other than isoprene. Such products may be excreted, secreted, or intracellular products. Any one of the methods described herein may be used to produce isoprene and/or one or more of the other industrial bio-products. Any one of the compositions and methods described herein may be used to produce isoprene and/or one or more other industrial bio-products that are derived from acetyl-CoA. The industrial bio-products described herein may be, for example, ethanol, propanediol (e.g., 1,2-propanediol, 1,3-propanediol), hydrogen, acetate, or microbial fuels. Exemplary microbial fuels are fermentative alcohols (e.g., ethanol or butanol), non-fermentative alcohols (e.g., isobutanol, methyl butanol, 1-propanol, 1-butanol, methyl pentanol, or 1-hexanol), fatty alcohols, fatty acid esters, isoprenoid alcohols, alkenes, and alkanes. The products described herein may also be a terpenoid, isoprenoid (e.g., farnesene), or carotenoid or other C5, C10, C15, C20, C25, C30, C35, or C40 product. The compounds that may be derived from acetyl-CoA (e.g., ethanol, isoprenoids, and fatty acids) are well-known in the art, including, for example, those described in WO 2013/3007786 and US 2012/0288891, the contents of which are expressly incorporated by reference in their entirety with respect to the polypeptides involved in pathways of producing ethanol, isoprenoids, and fatty acids.

In some aspects, the terpenoids are selected from the group consisting of hemiterpenoids, monoterpenoids, sesquiterpenoids, diterpenoids, sesterterpenoids, triterpenoids, tetraterpenoids, and higher polyterpenoids. In some aspects, the hemiterpenoid is prenol, isoprenol, or isovaleric acid. In some aspects, the monoterpenoid is geranyl pyrophosphate, eucalyptol, limonene, or pinene. In some aspects, the sesquiterpenoid is farnesyl pyrophosphate, artemisinin, or bisabolol. In some aspects, the diterpenoid is geranylgeranyl pyrophosphate, retinol, retinal, phytol, taxol, forskolin, or aphidicolin. In some aspects, the triterpenoid is squalene or lanosterol. In some aspects, the tetraterpenoid is lycopene or carotene. In some aspects, the carotenoids are selected from the group consisting of xanthophylls and carotenes. In some aspects, the xanthophyll is lutein or zeaxanthin. In some aspects, the carotene is α-carotene, β-carotene, γ-carotene, β-cryptoxanthin or lycopene.

The products described herein may be derived from Acetyl-CoA produced via syngas fermentation. In some aspects, the products described herein may be derived from Acetyl-CoA produced via carbohydrate fermentation. In other aspects, the products described herein may be derived from Acetyl-CoA and produced via fermentation of a combination of carbohydrate, hydrogen, and carbon dioxide. In some aspects, the cell is grown under conditions suitable for the production of the product(s) other than isoprene.

The products described herein may be naturally produced by the cell. In some aspects, the cells naturally produce one or more products including excreted, secreted, or intracellular products. In some aspects, the cells naturally produce ethanol, propanediol, hydrogen, or acetate. In some aspects, production of a naturally occurring product is increased relative to wild-type cells. Any method known in the art to increase production of a metabolic cellular product may be used to increase the production of a naturally occurring product. In some aspects, the nucleic acid encoding all or a part of the pathway for production of a product described herein is operably linked to a promoter such as a strong promoter. In some aspects, the nucleic acid encoding all or a part of the pathway for production of a product described herein is operably linked to a constitutive promoter. In some aspects, the cell is engineered to comprise additional copies of an endogenous nucleic acid encoding a polypeptide for the production of a product described herein. In some aspects, the product described herein is not naturally produced by the cell. In some aspects, the cell comprises one or more heterologous nucleic acids encoding one or more polypeptides for the production of a product described herein.

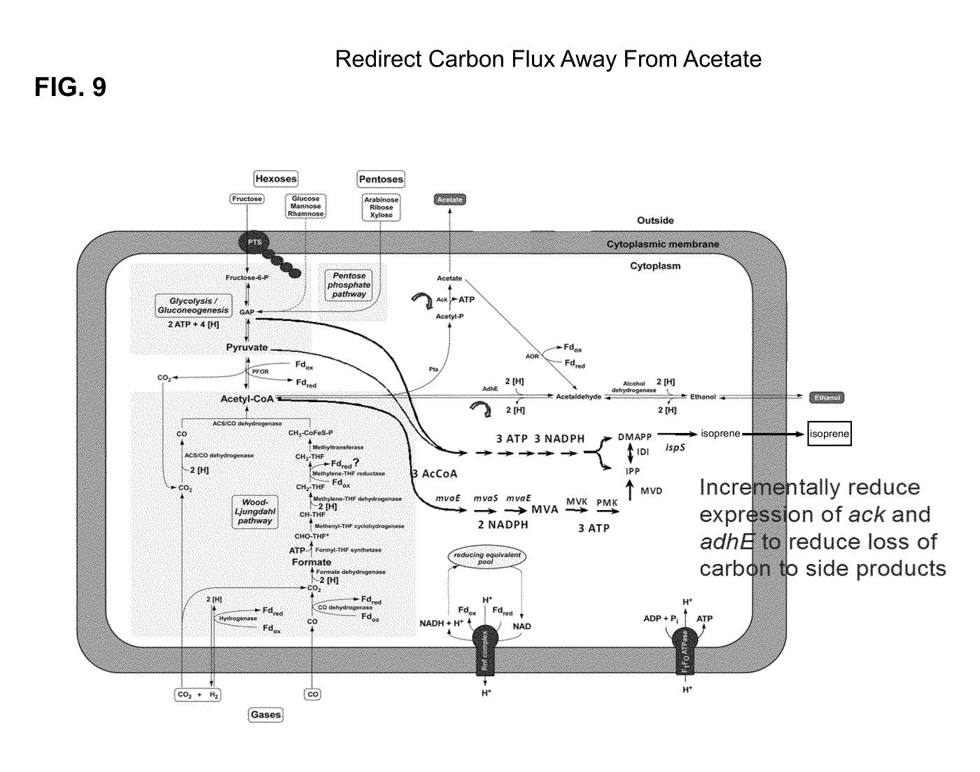

Under normal growth conditions, acetogens produce acetate and ethanol. Acetate is produced in a 2-step reaction in which acetyl-CoA is firstly converted to acetyl-phosphate by phosphotransacetylase (pta), then acetyl-phosphate is dephosphorylated by acetate kinase (ack) to form acetate. Ethanol is formed by a two step process in which acetyl-CoA is converted to acetaldehyde and then to ethanol by the multifunctional enzyme alcohol dehydrogenase (adhE). The production of acetate and ethanol may not be desirable in isoprene-producing cells, as it fluxes carbon away from isoprene and ultimately results in decreased yield of isoprene. Thus, some or all of the genes coding for phosphotransacetylase (pta), acetate kinase (ack), and alcohol dehydrogenase (adhE) may be disrupted or the expressions thereof are reduced in anaerobic cells for the purpose of redirecting carbon flux away from acetate and/or ethanol and increasing the production of isoprene. FIG. 9 shows a schematic representation of redirecting carbon flux away from acetate by reducing expression of ack and adhE to reduce loss of carbon to side products. The arrows next to Ack or AdhE used in the production of acetate and ethanol, respectively, indicate a reduction of activity or enzyme expression for pathways leading to fermentation products such as acetate, ethanol, or any other alcohol, or carbon containing end product. The purpose is to maximize carbon channeling to isoprene via genetic manipulation.

In some aspects, the cells are deficient in at least one polypeptide involved in production of acetate, ethanol, succinate, and/or glycerol. In some aspects, one or more pathways for production of a metabolite other than isoprene (e.g., lactate, acetate, ethanol (or other alcohol(s)), succinate, or glycerol) are blocked, for example, the production of a metabolite other than isoprene may be reduced by at least about any of 10%, 20%, 30%, 40%, 50%, 60%, 70%, 80%, 90%, or 95%. In some aspects, one or more of the pathways for production of lactate, acetate, ethanol, succinate, or glycerol is blocked, for example, the production for lactate, acetate, ethanol, succinate, and/or glycerol is reduced by at least about any of 10%, 20%, 30%, 40%, 50%, 60%, 70%, 80%, 90%, or 95%. In some aspects, the cells are deficient in at least one polypeptide in pathways(s) of producing acetate, ethanol, succinate, and/or glycerol. Polypeptides in pathways(s) of producing acetate, ethanol, succinate, and/or glycerol may have reduced activities or the expressions thereof are reduced. Nucleic acids encoding polypeptides in pathways(s) of producing acetate, ethanol, succinate, and/or glycerol may be disrupted. The polypeptides involved in various pathways (e.g., pathways for producing ethanol and/or acetate) are known to one skilled in the art, including, for example, those described in Misoph et al. 1996, J of Bacteriology, 178(11):3140-45, the contents of which are expressly incorporated by reference in its entirety with respect to the polypeptides involved in pathways of producing succinate, acetate, lactate, and/or ethanol.

In some aspects, the cells are deficient in pta. In some aspects, the cells are deficient in ack. In some aspects, the cells are deficient in adhE. In some aspects, the cells are deficient in pta, ack, and/or adhE. In some aspects, the expressions of phosphotransacetylase, acetate kinase, and/or alcohol dehydrogenase are reduced. In some aspects, the activities of phosphotransacetylase, acetate kinase, and/or alcohol dehydrogenase are reduced. In some aspects, the cells are deficient in polypeptide(s) having similar activities as phosphotransacetylase, acetate kinase, and/or alcohol dehydrogenase. The expression of pta, ack, adhE, and/or polypeptide(s) having similar activities as phosphotransacetylase, acetate kinase, and/or alcohol dehydrogenase may be reduced by any of the methods known to one skilled in the art, for example, the expression may be reduced by antisense RNA(s) (e.g., antisense RNA driven by any of the promoters described herein such as any of the inducible promoters). In some aspects, the antisense RNA(s) are operably linked to a suitable promoter such as any of the promoters described herein including inducible promoters.

In some aspects, production of acetate may be decreased during growth under conditions where synthesis gas is used as an energy and/or carbon source and carbohydrate is not added. Growth of Clostridium ljungdahlii on syngas is found to result in a two to five-fold decrease in acetate.

In some aspects, isoprene and product(s) other than isoprene are both recovered from the gas phase. In some aspects, isoprene is recovered from the gas phase (e.g. from the fermentation of gas), and the other product(s) are recovered from the liquid phase (e.g. from the cell broth).

In some embodiments, isoprene and other products such as industrial enzymes are produced. In other embodiments, the industrial enzyme is produced without the isoprene. Accordingly, in some embodiments, increased production of excreted, secreted and intracellular products such as isoprene and/or industrial enzymes are provided. Anaerobes as described herein can be used to produce industrial enzymes, which include, but are not limited to, hemicellulases, cellulases, peroxidases, proteases, metalloproteases, xylanases, lipases, phospholipases, esterases, perhydrolasess, cutinases, pectinases, pectate lyases, mannanases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. Exemplary protocols that can be used to make these industrial enzymes are disclosed in U.S. Appl. Pub. Nos. 2009/0311764, 2009/0275080, 2009/0252828, 2009/0226569, 2007/0259397, 2011/0027830, 2010/0015686, 2009/0253173, 2010/0055752, 2010/0196537, 2010/0021587, 2010/0221775, 2010/0304468, 2004/0014185, and U.S. Pat. Nos. 7,629,451; 7,604,974; 7,541,026; and 7,527,959, each of which is expressly incorporated in its entirety, particularly for materials, methods (including protocols) of production, recovery, and/or purification as well as the characteristics of the enzymes themselves.

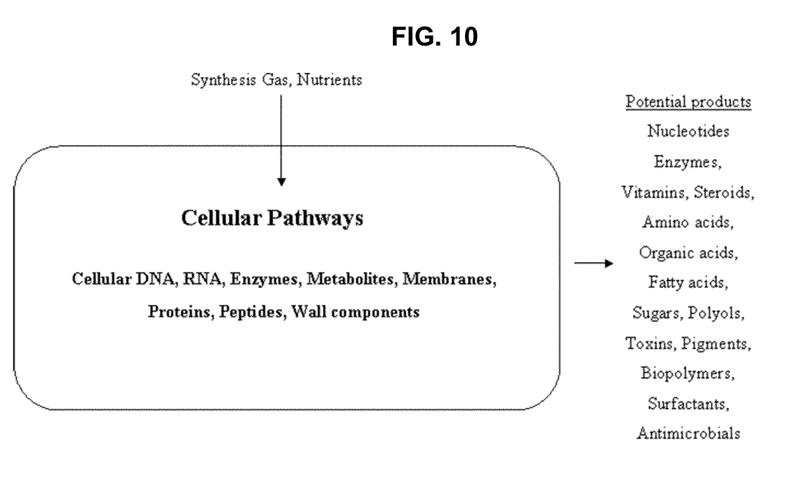

These obligate anaerobes can also be used to make nutraceuticals (such as vitamins, amino acids, nucleotides, sugars, etc., see, e.g., U.S. Pat. No. 7,622,290 which is expressly incorporated in its entirety, particularly for materials, methods (including protocols) of production, recovery, and/or purification as well as the characteristics of the nutraceuticals themselves), surfactants, antimicrobials (see, e.g., U.S. Appl Pub. No. 2009/0275103, which is expressly incorporated in its entirety, particularly for materials, methods (including protocols) of production, recovery, and/or purification as well as the characteristics of the antimicrobials themselves), biopolymers, organic acids (acetic acid, butyric acid, propionic acid, succinic acid, etc), bioplastic monomers (1,3-propanediol, lactic acid). Many of these compounds are synthesized from engineered pathway utilizing the building block of AcCoA via syngas fermentation. In some embodiments, a carbohydrate is used as energy and/or carbon source for the synthesis of these compounds. In other embodiments, a carbohydrate and hydrogen are used as energy and/or carbon source for the synthesis of these compounds. Pathways for production of these products are illustrated in FIG. 10.

Recovery Methods

In some aspects, any of the methods described herein further include recovering the isoprene. For example, the isoprene produced using the compositions and methods of the invention can be recovered using standard techniques. such as gas stripping, membrane enhanced separation, fractionation, adsorption/desorption, pervaporation, thermal or vacuum desorption of isoprene from a solid phase, or extraction of isoprene immobilized or absorbed to a solid phase with a solvent (see, for example, U.S. Pat. Nos. 4,703,007 and 4,570,029). In one aspect, the isoprene is recovered by absorption stripping (see, for example, International Patent Application No. PCT/US2010/060552 (WO 2011/075534)). In particular aspects, extractive distillation with an alcohol (such as ethanol, methanol, propanol, or a combination thereof) is used to recover the isoprene. In some aspects, the recovery of isoprene involves the isolation of isoprene in a liquid form (such as a neat solution of isoprene or a solution of isoprene in a solvent). Gas stripping involves the removal of isoprene vapor from the fermentation off-gas stream in a continuous manner. Such removal can be achieved in several different ways including, but not limited to, adsorption to a solid phase, partition into a liquid phase, or direct condensation (such as condensation due to exposure to a condensation coil or do to an increase in pressure). In some aspects, membrane enrichment of a dilute isoprene vapor stream above the dew point of the vapor resulting in the condensation of liquid isoprene. In some aspects, the isoprene is compressed and condensed.

The recovery of isoprene may involve one step or multiple steps. In some aspects, the removal of isoprene vapor from the fermentation off-gas and the conversion of isoprene to a liquid phase are performed simultaneously. For example, isoprene can be directly condensed from the off-gas stream to form a liquid. In some aspects, the removal of isoprene vapor from the fermentation off-gas and the conversion of isoprene to a liquid phase are performed sequentially. For example, isoprene may be adsorbed to a solid phase and then extracted from the solid phase with a solvent.

Isoprene compositions recovered from fermentations in anaerobic organisms may contain impurities. The identities and levels of impurities in an isoprene composition can be analyzed by standard methods, such as GC/MS, GC/FID, and ¹H NMR. An impurity can be of microbial origin, or it can be a contaminant in the synthesis gas feed or other fermentation raw materials.

In some aspects, the isoprene composition recovered from fermentation in an anaerobic organism comprises one or more of the following impurities: hydrogen sulfide, carbonyl sulfide, carbon disulfide, ethanol, acetone, methanol, acetaldehyde, methacrolein, methyl vinyl ketone, 2-methyl-2-vinyloxirane, cis- and trans-3-methyl-1,3-pentadiene, a C5 prenyl alcohol (such as 3-methyl-3-buten-1-ol or 3-methyl-2-buten-1-ol), 2-heptanone, 6-methyl-5-hepten-2-one, 2,4,5-trimethylpyridine, 2,3,5-trimethylpyrazine, citronellal, methanethiol, ethanethiol, methyl acetate, 1-propanol, diacetyl, 2-butanone, 2-methyl-3-buten-2-ol, ethyl acetate, 2-methyl-1-propanol, 3-methyl-1-butanal, 3-methyl-2-butanone, 1-butanol, 2-pentanone, 3-methyl-1-butanol, ethyl isobutyrate, 3-methyl-2-butenal, butyl acetate, 3-methylbutyl acetate, 3-methyl-3-buten-1-yl acetate, 3-methyl-2-buten-1-yl acetate, (E)-3,7-dimethyl-1,3,6-octatriene, (Z)-3,7-dimethyl-1,3,6-octatriene, (E,E)-3,7,11-trimethyl-1,3,6,10-dodecatetraene and (E)-7,11-dimethyl-3-methylene-1,6,10-dodecatriene, 3-hexen-1-ol, 3-hexen-1-yl acetate, limonene, geraniol (trans-3,7-dimethyl-2,6-octadien-1-ol), citronellol (3,7-dimethyl-6-octen-1-ol), (E)-3-methyl-1,3-pentadiene, (Z)-3-methyl-1,3-pentadiene, thiol(s), mono and disulfide(s), or gas(es) such as CS₂ and COS. The isoprene composition recovered from syngas fermentation in an anaerobic organism may comprise one or more of the components described in Rimbault A et al. 1986, J of Chromatography, 375:11-25, the contents of which are expressly incorporated herein by reference in its entirety with respect to various components in gases of Clostridium cultures.

In some aspects, any of the methods described herein further include purifying the isoprene. For example, the isoprene produced using the compositions and methods of the invention can be purified using standard techniques. Purification refers to a process through which isoprene is separated from one or more components that are present when the isoprene is produced. In some aspects, the isoprene is obtained as a substantially pure liquid. Examples of purification methods include (i) distillation from a solution in a liquid extractant and (ii) chromatography. As used herein, “purified isoprene” means isoprene that has been separated from one or more components that are present when the isoprene is produced. In some aspects, the isoprene is at least about 20%, by weight, free from other components that are present when the isoprene is produced. In various aspects, the isoprene is at least or about 25%, 30%, 40%, 50%, 60%, 70%, 75%, 80%, 90%, 95%, or 99%, by weight, pure. Purity can be assayed by any appropriate method, e.g., by column chromatography, HPLC analysis, or GC-MS analysis.

In some aspects, at least a portion of the gas phase remaining after one or more recovery steps for the removal of isoprene is recycled by introducing the gas phase into a cell culture system (such as a fermentor) for the production of isoprene.

In some embodiments, recovery of industrial enzymes can use any method known to one of skill in the art and/or any of the exemplary protocols that are disclosed in U.S. Appl. Pub. Nos. 2009/0311764, 2009/0275080, 2009/0252828, 2009/0226569, 2007/0259397 and U.S. Pat. Nos. 7,629,451; 7,604,974; 7,541,026; and 7,527,959 and for neutraceuticals (see, e.g., U.S. Pat. No. 7,622,290), and for antimicrobials (see, e.g., U.S. Appl Pub. No. 2009/0275103).

### Exemplary Embodiments

The invention provides for compositions of obligate anaerobic organisms (e.g., microorganisms or cells) which have been engineered to produce isoprene and/or other industrial bio-products using carbohydrate or carbohydrate combined with hydrogen and carbon dioxide as carbon and/or energy sources. Methods of making and using such organisms for the production of isoprene and/or other industrial bioproducts are also provided.

Accordingly, in some embodiments, the invention provides obligate anaerobic cells capable of producing isoprene, said cells comprising one or more heterologous nucleic acids encoding an isoprene synthase polypeptide in operable combination with a promoter, wherein the culturing of said cells under substantially oxygen-free culture conditions comprising a carbohydrate carbon source provides for the production of isoprene.

In any of the embodiments described herein, the cells are selected from the group consisting of Clostridium ljungdahlii, Clostridium aceticum, Clostridium acetobutylicum, Moorella thermoacetica, Clostridium autoethanogenum, Eurobacterium limosum, Clostridium carboxydivorans, Peptostreptococcus productus, Rhodospirillum rubrum, Desulfitobacterium hafniense, Aecetoanaerobium notera, Butyribacterium methylotrophicum, Thermoanaerobacter kivui, Eubacterium limosum, Peptostreptococcus productus, and Acetobacterium woodi.

In any of the embodiments described herein, the cells are Clostridium cells. In any of the embodiments described herein, the cells are selected from the group consisting of Clostridium ljungdahlii, Clostridium aceticum, Clostridium acetobutylicum, Clostridium carboxidivorans, and Clostridium autoethanogenum. In any of the embodiments described herein, said promoter is an inducible promoter or a constitutive promoter.

In any of the embodiments described herein, said isoprene synthase polypeptide is a plant isoprene synthase polypeptide or a variant thereof. In any of the embodiments described herein, the plant isoprene synthase polypeptide is an isoprene synthase from Pueraria or a variant thereof. In any of the embodiments described herein, the plant isoprene synthase polypeptide is an isoprene synthase from Populus or a variant thereof. In any of the embodiments described herein, the plant isoprene synthase polypeptide is an isoprene synthase from a hybrid Populus alba×Populus tremula or a variant thereof. In any of the embodiments described herein, the plant isoprene synthase polypeptide is a poplar isoprene synthase polypeptide or a variant thereof. In any of the embodiments described herein, the plant isoprene synthase polypeptide is a kudzu isoprene synthase polypeptide or a variant thereof. In any of the embodiments described herein, the plant isoprene synthase polypeptide is an isoprene synthase from Pueraria montana, Pueraria lobata, Populus tremuloides, Populus alba, Populus nigra, or Populus trichocarpa or a variant thereof. In any of the embodiments described herein, the plant isoprene synthase polypeptide is an isoprene synthase from Populus alba or a variant thereof. In any of the embodiments described herein, the isoprene synthase polypeptide is a variant of a naturally occurring isoprene synthase. In any of the embodiments described herein, the isoprene synthase polypeptide is a variant of a naturally occurring isoprene synthase and has improved activity compared to a naturally occurring isoprene synthase.

In any of the embodiments described herein, the cells are deficient in protease such that the isoprene synthase polypeptide is not degraded or more resistant to degradation compared to cells that are not deficient in the protease.

In any of the embodiments described herein, the cells further comprise one or more heterologous nucleic acids encoding one or more mevalonate (MVA) pathway polypeptide(s). In any of the embodiments described herein, said one or more heterologous nucleic acids encoding one or more mevalonate (MVA) pathway polypeptides is a heterologous nucleic acid encoding an upper mevalonate (MVA) pathway polypeptide and/or a lower MVA pathway polypeptide. In any of the embodiments described herein, the upper MVA pathway polypeptide is selected from the group consisting of: (i) acetoacetyl-Coenzyme A synthase (thiolase) polypeptide; (ii) 3-hydroxy-3-methylglutaryl-Coenzyme A synthase polypeptide; and (iii) 3-hydroxy-3-methylglutaryl-Coenzyme A reductase polypeptide. In any of the embodiments described herein, the lower MVA pathway polypeptide is selected from the group consisting of: (i) mevalonate kinase (MVK); (ii) phosphomevalonate kinase (PMK); (iii) diphosphomevalonate decarboxylase (MVD); and (iv) isopentenyl diphosphate isomerase (IDI). In any of the embodiments described herein, the upper MVA pathway polypeptides are encoded nucleic acids encoding an mvaE polypeptide and an mvaS polypeptide. In any of the embodiments described herein, the IDI polypeptide is a yeast IDI polypeptide. In any of the embodiments described herein, the cells further comprise one or more nucleic acids encoding DXP pathway polypeptide(s). In any of the embodiments described herein, the DXP pathway polypeptide is DXS.

In any of the embodiments described herein, at least one pathway for production of a metabolite other than isoprene is blocked. In any of the embodiments described herein, one or more of the pathways for production of lactate, acetate, ethanol, succinate, or glycerol is blocked.

In other aspects, the invention features obligate anaerobic cells capable of producing isoprenoid precursors, said cells comprising one or more heterologous nucleic acids encoding one or more mevalonate (MVA) pathway polypeptides in operable combination with a promoter, wherein the culturing of said cells under substantially oxygen-free culture conditions comprising a carbohydrate carbon source provides for the production of isoprenoid precursors.

In any of the embodiments described herein, the cells are selected from the group consisting of Clostridium ljungdahlii, Clostridium aceticum, Clostridium acetobutylicum, Moorella thermoacetica, Clostridium autoethanogenum, Eurobacterium limosum, Clostridium carboxydivorans, Peptostreptococcus productus, Rhodospirillum rubrum, Desulfitobacterium hafniense, Aecetoanaerobium notera, Butyribacterium methylotrophicum, Thermoanaerobacter kivui, Eubacterium limosum, Peptostreptococcus productus, and Acetobacterium woodi. In any of the embodiments described herein, the cells are Clostridium cells.

In any of the embodiments described herein, the cells are selected from the group consisting of Clostridium ljungdahlii, Clostridium aceticum, Clostridium acetobutylicum, Clostridium carboxidivorans, and Clostridium autoethanogenum. In any of the embodiments described herein, said promoter is an inducible promoter or constitutive promoter.

In any of the embodiments described herein, said one or more heterologous nucleic acids encoding one or more mevalonate (MVA) pathway polypeptides is a heterologous nucleic acid encoding an upper mevalonate (MVA) pathway polypeptide and/or a lower MVA pathway polypeptide. In any of the embodiments described herein, the upper MVA pathway polypeptide is selected from the group consisting of: (i) acetoacetyl-Coenzyme A synthase (thiolase) polypeptide; (ii) 3-hydroxy-3-methylglutaryl-Coenzyme A synthase polypeptide; and (iii) 3-hydroxy-3-methylglutaryl-Coenzyme A reductase polypeptide. In any of the embodiments described herein, the lower MVA pathway polypeptide is selected from the group consisting of: (i) mevalonate kinase (MVK); (ii) phosphomevalonate kinase (PMK); (iii) diphosphomevalonate decarboxylase (MVD); and (iv) isopentenyl diphosphate isomerase (IDI). In any of the embodiments described herein, the upper MVA pathway polypeptides are encoded nucleic acids encoding an mvaE polypeptide and an mvaS polypeptide. In any of the embodiments described herein, said isoprenoid precursor is selected from the groups consisting of MVA, IPP, and DMAPP.

In another aspect, the invention features obligate anaerobic cells capable of producing isoprenoids, said cells comprising: (a) one or more heterologous nucleic acids encoding a polyprenyl pyrophosphate synthase polypeptide in operable combination with a promoter; and (b) one or more heterologous nucleic acids encoding one or more mevalonate (MVA) pathway polypeptides in operable combination with a promoter, wherein the culturing of said cells under substantially oxygen-free culture conditions comprising a carbohydrate carbon source provides for the production of isoprenoids.

In any of the embodiments described herein, the cells are selected from the group consisting of Clostridium ljungdahlii, Clostridium aceticum, Clostridium acetobutylicum, Moorella thermoacetica, Clostridium autoethanogenum, Eurobacterium limosum, Clostridium carboxydivorans, Peptostreptococcus productus, Rhodospirillum rubrum, Desulfitobacterium hafniense, Aecetoanaerobium notera, Butyribacterium methylotrophicum, Thermoanaerobacter kivui, Eubacterium limosum, Peptostreptococcus productus, and Acetobacterium woodi. In any of the embodiments described herein, the cells are Clostridium cells. In any of the embodiments described herein, the cells are selected from the group consisting of Clostridium ljungdahlii, Clostridium aceticum, Clostridium acetobutylicum, Clostridium carboxidivorans, and Clostridium autoethanogenum. In any of the embodiments described herein, said promoter is an inducible promoter or a constitutive promoter.

In any of the embodiments described herein, said one or more heterologous nucleic acids encoding one or more mevalonate (MVA) pathway polypeptides is a heterologous nucleic acid encoding an upper mevalonate (MVA) pathway polypeptide and/or a lower MVA pathway polypeptide. In any of the embodiments described herein, the upper MVA pathway polypeptide is selected from the group consisting of: (i) acetoacetyl-Coenzyme A synthase (thiolase) polypeptide; (ii) 3-hydroxy-3-methylglutaryl-Coenzyme A synthase polypeptide; and (iii) 3-hydroxy-3-methylglutaryl-Coenzyme A reductase polypeptide. In any of the embodiments described herein, the lower MVA pathway polypeptide is selected from the group consisting of: (i) mevalonate kinase (MVK); (ii) phosphomevalonate kinase (PMK); (iii) diphosphomevalonate decarboxylase (MVD); and (iv) isopentenyl diphosphate isomerase (IDI). In any of the embodiments described herein, the upper MVA pathway polypeptides are encoded nucleic acids encoding an mvaE polypeptide and an mvaS polypeptide.

In any of the embodiments described herein, the isoprenoid is selected from group consisting of monoterpenes, diterpenes, triterpenes, tetraterpenes, sesquiterpene, and polyterpene. In any of the embodiments described herein, the isoprenoid is a sesquiterpene. In any of the embodiments described herein, the isoprenoid is selected from the group consisting of abietadiene, amorphadiene, carene, α-farnesene, β-farnesene, farnesol, geraniol, geranylgeraniol, linalool, limonene, myrcene, nerolidol, ocimene, patchoulol, β-pinene, sabinene, γ-terpinene, terpinene and valencene.

In other aspects, the invention features obligate anaerobic cells capable of producing acetyl-CoA derived products, said cells comprising one or more heterologous nucleic acids encoding a polypeptide involved in the conversion of acetyl-CoA into a acetyl-CoA derived product in operable combination with a promoter, wherein the culturing of said cells under substantially oxygen-free culture conditions comprising a carbohydrate carbon source provides for the production of said acetyl-CoA derived product.

In any of the embodiments described herein, the acetyl-CoA derived product is selected from the group consisting of 2-keto acids, malonyl-CoA, acetoacetyl-CoA and/or ethanol. In any of the embodiments described herein, the cells further comprise: (a) one or more heterologous nucleic acids encoding a one or more polypeptides capable of converting a 2-keto acid into a non-fermentative alcohol; (b) one or more heterologous nucleic acids encoding one or more polypeptides capable of converting malonyl-CoA into a fatty acid-derived hydrocarbon; or (c) one or more heterologous nucleic acids encoding one or more polypeptides capable of converting acetoacetyl-CoA into a fermentative alcohol. In any of the embodiments described herein, said non-fermentative alcohol is selected from the group consisting of 1-propanol, 1-butanol, isobutanol, 2-methyl-1-butanol, 3-methyl-1-butanol, 3-methyl-1-pentanol, 4-methyl-1-pentanol and 1-hexanol. In any of the embodiments described herein, said fatty acid-derived hydrocarbon is selected from the group consisting of fatty alcohols, fatty esters, olefins, and alkanes. In any of the embodiments described herein, said fermentative alcohol is butanol.

In other aspects, the invention features a method for producing isoprene comprising the steps of: (a) culturing obligate anaerobic cells comprising one or more heterologous nucleic acids encoding isoprene synthase polypeptide in substantially oxygen-free culture conditions comprising a carbohydrate carbon source; and (b) producing said isoprene.

In other aspects, the invention features a method for producing isoprene comprising the steps of: (a) culturing obligate anaerobic cells comprising one or more heterologous nucleic acids encoding isoprene synthase polypeptide and/or one or more mevalonate pathway polypeptides in substantially oxygen-free culture conditions comprising a carbohydrate carbon source; and (b) producing said isoprene.

In other aspects, the invention features a method for producing isoprenoid precursors comprising the steps of: (a) culturing obligate anaerobic cells comprising one or more heterologous nucleic acids encoding one or more mevalonate (MVA) pathway polypeptides in operable combination with a promoter under substantially oxygen-free culture conditions comprising a carbohydrate carbon source; and (b) producing said isoprenoid precursors.

In other aspects, the invention features a method for producing an acetyl-CoA derived product comprising the steps of: (a) culturing obligate anaerobic cells comprising one or more heterologous nucleic acids encoding a polypeptide involved in the conversion of acetyl-CoA into an acetyl-CoA derived product in operable combination with a promoter in substantially oxygen-free culture conditions comprising a carbohydrate carbon source; and (b) producing a fermentative alcohol, fatty acid-derived hydrocarbon, or a fermentative alcohol product.

In any of the embodiments of the methods described herein, the method further comprises recovering the isoprene. In any of the embodiments described herein, the isoprene is recovered by absorption stripping. In any of the embodiments of the methods described herein, the method further comprises recovering the isoprenoid. In any of the embodiments described herein, the isoprenoid is recovered from the liquid phase. In any of the embodiments of the methods described herein, the method further comprises recovering the fermentative alcohol, fatty acid-derived hydrocarbon, or fermentative alcohol product.

In some embodiments, the invention provides obligate anaerobic cells capable of increased production of isoprene, said cells comprising one or more heterologous nucleic acids encoding an isoprene synthase polypeptide in operable combination with a promoter, wherein culturing of said cells under substantially oxygen-free culture conditions comprising carbohydrate and hydrogen as carbon and/or energy sources provides for increased production of isoprene as compared to said cells being cultured in the presence of carbohydrate alone.

In any of the embodiments described herein, the cells are selected from the group consisting of Clostridium ljungdahlii, Clostridium aceticum, Clostridium acetobutylicum, Moorella thermoacetica, Clostridium autoethanogenum, Eurobacterium limosum, Clostridium carboxydivorans, Peptostreptococcus productus, Rhodospirillum rubrum, Desulfitobacterium hafniense, Aecetoanaerobium notera, Butyribacterium methylotrophicum, Thermoanaerobacter kivui, Eubacterium limosum, Peptostreptococcus productus, and Acetobacterium woodi.

In any of the embodiments described herein, the cells are Clostridium cells. In any of the embodiments described herein, the cells are selected from the group consisting of Clostridium ljungdahlii, Clostridium aceticum, Clostridium acetobutylicum, Clostridium carboxidivorans, and Clostridium autoethanogenum. In any of the embodiments described herein, said promoter is an inducible promoter or a constitutive promoter.

In any of the embodiments described herein, said isoprene synthase polypeptide is a plant isoprene synthase polypeptide or a variant thereof. In any of the embodiments described herein, the plant isoprene synthase polypeptide is an isoprene synthase from Pueraria or a variant thereof. In any of the embodiments described herein, the plant isoprene synthase polypeptide is an isoprene synthase from Populus or a variant thereof. In any of the embodiments described herein, the plant isoprene synthase polypeptide is an isoprene synthase from a hybrid Populus alba×Populus tremula or a variant thereof. In any of the embodiments described herein, the plant isoprene synthase polypeptide is a poplar isoprene synthase polypeptide or a variant thereof. In any of the embodiments described herein, the plant isoprene synthase polypeptide is a kudzu isoprene synthase polypeptide or a variant thereof. In any of the embodiments described herein, the plant isoprene synthase polypeptide is an isoprene synthase from Pueraria montana, Pueraria lobata, Populus tremuloides, Populus alba, Populus nigra, or Populus trichocarpa or a variant thereof. In any of the embodiments described herein, the plant isoprene synthase polypeptide is an isoprene synthase from Populus alba or a variant thereof. In any of the embodiments described herein, the isoprene synthase polypeptide is a variant of a naturally occurring isoprene synthase. In some embodiments, the isoprene synthase polypeptide is a variant of a naturally occurring isoprene synthase and has improved activity compared to a naturally occurring isoprene synthase.

In any of the embodiments described herein, the cells are deficient in protease such that the isoprene synthase polypeptide is not degraded or more resistant to degradation compared to cells that are not deficient in the protease.

In any of the embodiments described herein, the cells further comprise one or more heterologous nucleic acids encoding one or more mevalonate (MVA) pathway polypeptide(s). In any of the embodiments described herein, said one or more heterologous nucleic acids encoding one or more mevalonate (MVA) pathway polypeptides is a heterologous nucleic acid encoding an upper mevalonate (MVA) pathway polypeptide and/or a lower MVA pathway polypeptide. In any of the embodiments described herein, the upper MVA pathway polypeptide is selected from the group consisting of: (i) acetoacetyl-Coenzyme A synthase (thiolase) polypeptide; (ii) 3-hydroxy-3-methylglutaryl-Coenzyme A synthase polypeptide; and (iii) 3-hydroxy-3-methylglutaryl-Coenzyme A reductase polypeptide. In any of the embodiments described herein, the lower MVA pathway polypeptide is selected from the group consisting of: (i) mevalonate kinase (MVK); (ii) phosphomevalonate kinase (PMK); (iii) diphosphomevalonate decarboxylase (MVD); and (iv) isopentenyl diphosphate isomerase (IDI). In any of the embodiments described herein, the upper MVA pathway polypeptides are encoded nucleic acids encoding an mvaE polypeptide and an mvaS polypeptide. In any of the embodiments described herein, the IDI polypeptide is a yeast IDI polypeptide. In any of the embodiments described herein, the cells further comprise one or more nucleic acids encoding DXP pathway polypeptide(s). In any of the embodiments described herein, the DXP pathway polypeptide is DXS.

In any of the embodiments described herein, at least one pathway for production of a metabolite other than isoprene is blocked. In any of the embodiments described herein, one or more of the pathways for production of lactate, acetate, ethanol, succinate, or glycerol is blocked.

In other aspects, the invention features obligate anaerobic cells capable of increased production of isoprenoid precursors, said cells comprising one or more heterologous nucleic acids encoding one or more mevalonate (MVA) pathway polypeptides in operable combination with a promoter, wherein culturing said cells under substantially oxygen-free culture conditions comprising carbohydrate and hydrogen as carbon and/or energy sources provides for increased production of isoprenoid precursors as compared to said cells cultured in the presence of carbohydrate alone.

In any of the embodiments described herein, the cells are selected from the group consisting of Clostridium ljungdahlii, Clostridium aceticum, Clostridium acetobutylicum, Moorella thermoacetica, Clostridium autoethanogenum, Eurobacterium limosum, Clostridium carboxydivorans, Peptostreptococcus productus, Rhodospirillum rubrum, Desulfitobacterium hafniense, Aecetoanaerobium notera, Butyribacterium methylotrophicum, Thermoanaerobacter kivui, Eubacterium limosum, Peptostreptococcus productus, and Acetobacterium woodi. In any of the embodiments described herein, the cells are Clostridium cells.

In any of the embodiments described herein, the cells are selected from the group consisting of Clostridium ljungdahlii, Clostridium aceticum, Clostridium acetobutylicum, Clostridium carboxidivorans, and Clostridium autoethanogenum. In any of the embodiments described herein, said promoter is an inducible promoter or constitutive promoter.

In any of the embodiments described herein, said one or more heterologous nucleic acids encoding one or more mevalonate (MVA) pathway polypeptides is a heterologous nucleic acid encoding an upper mevalonate (MVA) pathway polypeptide and/or a lower MVA pathway polypeptide. In any of the embodiments described herein, the upper MVA pathway polypeptide is selected from the group consisting of: (i) acetoacetyl-Coenzyme A synthase (thiolase) polypeptide; (ii) 3-hydroxy-3-methylglutaryl-Coenzyme A synthase polypeptide; and (iii) 3-hydroxy-3-methylglutaryl-Coenzyme A reductase polypeptide. In any of the embodiments described herein, the lower MVA pathway polypeptide is selected from the group consisting of: (i) mevalonate kinase (MVK); (ii) phosphomevalonate kinase (PMK); (iii) diphosphomevalonate decarboxylase (MVD); and (iv) isopentenyl diphosphate isomerase (IDI). In any of the embodiments described herein, the upper MVA pathway polypeptides are encoded nucleic acids encoding an mvaE polypeptide and an mvaS polypeptide. In any of the embodiments described herein, said isoprenoid precursor is selected from the groups consisting of MVA, IPP, and DMAPP.

In other aspects, the invention features obligate anaerobic cells capable of increased production of isoprenoids, said cells comprising: (a) one or more heterologous nucleic acids encoding a polyprenyl pyrophosphate synthase polypeptide in operable combination with a promoter; and (b) one or more heterologous nucleic acids encoding one or more mevalonate (MVA) pathway polypeptides in operable combination with a promoter, wherein the culturing of said cells under substantially oxygen-free culture conditions comprising carbohydrate and hydrogen as carbon and/or energy sources provides for increased production of isoprenoids as compared to said cells cultured in the presence of carbohydrate alone.

In any of the embodiments described herein, the cells are selected from the group consisting of Clostridium ljungdahlii, Clostridium aceticum, Clostridium acetobutylicum, Moorella thermoacetica, Clostridium autoethanogenum, Eurobacterium limosum, Clostridium carboxydivorans, Peptostreptococcus productus, Rhodospirillum rubrum, Desulfitobacterium hafniense, Aecetoanaerobium notera, Butyribacterium methylotrophicum, Thermoanaerobacter kivui, Eubacterium limosum, Peptostreptococcus productus, and Acetobacterium woodi. In any of the embodiments described herein, the cells are Clostridium cells. In any of the embodiments described herein, the cells are selected from the group consisting of Clostridium ljungdahlii, Clostridium aceticum, Clostridium acetobutylicum, Clostridium carboxidivorans, and Clostridium autoethanogenum. In any of the embodiments described herein, said promoter is an inducible promoter or a constitutive promoter.

In any of the embodiments described herein, said one or more heterologous nucleic acids encoding one or more mevalonate (MVA) pathway polypeptides is a heterologous nucleic acid encoding an upper mevalonate (MVA) pathway polypeptide and/or a lower MVA pathway polypeptide. In any of the embodiments described herein, the upper MVA pathway polypeptide is selected from the group consisting of: (i) acetoacetyl-Coenzyme A synthase (thiolase) polypeptide; (ii) 3-hydroxy-3-methylglutaryl-Coenzyme A synthase polypeptide; and (iii) 3-hydroxy-3-methylglutaryl-Coenzyme A reductase polypeptide. In any of the embodiments described herein, the lower MVA pathway polypeptide is selected from the group consisting of: (i) mevalonate kinase (MVK); (ii) phosphomevalonate kinase (PMK); (iii) diphosphomevalonate decarboxylase (MVD); and (iv) isopentenyl diphosphate isomerase (IDI). In any of the embodiments described herein, the upper MVA pathway polypeptides are encoded nucleic acids encoding an mvaE polypeptide and an mvaS polypeptide.

In any of the embodiments described herein, the isoprenoid is selected from group consisting of monoterpenes, diterpenes, triterpenes, tetraterpenes, sesquiterpene, and polyterpene. In any of the embodiments described herein, the isoprenoid is a sesquiterpene. In any of the embodiments described herein, the isoprenoid is selected from the group consisting of abietadiene, amorphadiene, carene, α-farnesene, β-farnesene, farnesol, geraniol, geranylgeraniol, linalool, limonene, myrcene, nerolidol, ocimene, patchoulol, β-pinene, sabinene, γ-terpinene, terpinene and valencene.

In other aspects, the invention features obligate anaerobic cells capable of increased production of acetyl-CoA derived products, said cells comprising one or more heterologous nucleic acids encoding a polypeptide involved in the conversion of acetyl-CoA into a acetyl-CoA derived product in operable combination with a promoter, wherein the culturing of said cells under substantially oxygen-free culture conditions comprising carbohydrate and hydrogen as carbon and/or energy sources provides for increased production of said acetyl-CoA derived product as compared to said cells cultured in the presence of carbohydrate alone.

In any of the embodiments described herein, the acetyl-CoA derived product is selected from the group consisting of 2-keto acids, malonyl-CoA, acetoacetyl-CoA and/or ethanol. In any of the embodiments described herein, the cells further comprise: (a) one or more heterologous nucleic acids encoding one or more polypeptides capable of converting a 2-keto acid into a non-fermentative alcohol; (b) one or more heterologous nucleic acids encoding one or more polypeptides capable of converting malonyl-CoA into a fatty acid-derived hydrocarbon; or (c) one or more heterologous nucleic acids encoding one or more polypeptides capable of converting acetoacetyl-CoA into a fermentative alcohol. In any of the embodiments described herein, said non-fermentative alcohol is selected from the group consisting of 1-propanol, 1-butanol, isobutanol, 2-methyl-1-butanol, 3-methyl-1-butanol, 3-methyl-1-pentanol, 4-methyl-1-pentanol and 1-hexanol. In any of the embodiments described herein, said fatty acid-derived hydrocarbon is selected from the group consisting of fatty alcohols, fatty esters, olefins, and alkanes. In any of the embodiments described herein, said fermentative alcohol is butanol.

In other aspects, the invention features a method for increased production of isoprene comprising the steps of: (a) culturing obligate anaerobic cells comprising one or more heterologous nucleic acids encoding isoprene synthase polypeptide in substantially oxygen-free culture conditions comprising carbohydrate and hydrogen as carbon and/or energy sources; and (b) producing said isoprene, wherein said method provides for increased production of isoprene as compared to culturing said cells in the presence of carbohydrate alone.

In other aspects, the invention features a method for increased production of isoprene comprising the steps of: (a) culturing obligate anaerobic cells comprising one or more heterologous nucleic acids encoding isoprene synthase polypeptide and/or one or more mevalonate pathway polypeptides in substantially oxygen-free culture conditions comprising carbohydrate and hydrogen as carbon and/or energy sources; and (b) producing said isoprene, wherein said method provides for increased production of isoprene as compared to culturing said cells in the presence of carbohydrate alone.

In other aspects, the invention features a method for increased production of isoprenoid precursors comprising the steps of: (a) culturing obligate anaerobic cells comprising one or more heterologous nucleic acids encoding one or more mevalonate (MVA) pathway polypeptides in operable combination with a promoter under substantially oxygen-free culture conditions comprising carbohydrate and hydrogen as carbon and/or energy sources; and (b) producing said isoprenoid precursors, wherein said method provides for increased production of isoprenoid precursors as compared to culturing said cells in the presence of carbohydrate alone.

In other aspects, the invention features a method for increased production of an isoprenoid comprising the steps of: (a) culturing obligate anaerobic cells comprising one or more heterologous nucleic acids encoding one or more mevalonate (MVA) pathway polypeptides in operable combination with a promoter under substantially oxygen-free culture conditions comprising carbohydrate and hydrogen as carbon and/or energy sources; and (b) producing said isoprenoid, wherein said method provides for increased production of isoprenoid as compared to culturing said cells in the presence of carbohydrate alone.

In other aspects, the invention features a method for increased production of acetyl-CoA derived products comprising the steps of: (a) culturing obligate anaerobic cells comprising one or more heterologous nucleic acids encoding a polypeptide involved in the conversion of acetyl-CoA into an acetyl-CoA derived product in operable combination with a promoter in substantially oxygen-free culture conditions comprising carbohydrate and hydrogen as carbon and/or energy sources; and (b) producing a fermentative alcohol, fatty acid-derived hydrocarbon, or a fermentative alcohol product, wherein said method provides for increased production of acetyl-CoA derived products as compared to culturing said cells in the presence of carbohydrate alone.

In any of the embodiments of the methods described herein, the method further comprises recovering the isoprene. In any of the embodiments described herein, the isoprene is recovered by absorption stripping. In any of the embodiments of the methods described herein, the method further comprises recovering the isoprenoid. In any of the embodiments described herein, the isoprenoid is recovered from the liquid phase. In any of the embodiments of the methods described herein, the method further comprises recovering the fermentative alcohol, fatty acid-derived hydrocarbon, or fermentative alcohol product.

The following examples have been provided for illustrative purposes only and are not intended to limit the invention.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “kb” means kilobase(s), “bp” means base pairs, “nt” means nucleotide(s), “hr” or “h” means hour(s), “min” means minute(s), “sec” means second(s), “d” means day(s), “L” means liter(s), “ml” or “mL” means milliliter(s), “μL” means microliter(s), “μg” means microgram(s), “g” means gram(s), “mg” means milligram(s), “mM” means millimolar, “μM” means micromolar, “M” means molar, “wt” means weight, “f.” means formula, “V” means volts, “Ω” means resistance in ohms, “μF” means capacitance in microfarads, “WT” means wild type, “OD₆₀₀” means optical density at 600 nm.

### General Methods

Expression and Purification of Recombinant MvaE and MvaS from E. Faecalis

E. coli cells expressing his tagged E. faecalis MvaS polypeptide (SEQ ID NO:3) were grown in Luria-Bertani broth supplemented with 50 mg/L carbenicillin and 30 mg/L chloramphenicol. They were induced overnight with the addition of 0.2 mM IPTG at an OD600 of ˜0.4-0.6. E. coli cells expressing his tagged E. faecalis MvaE polypeptide (SEQ ID NO:4) were grown in Terrific broth supplemented with 50 mg/L kanamycin and were induced for 6 hours with the addition of 0.2 mM IPTG at OD(600) equal to 1.0. All cells were harvested by centrifugation at 10,000×g for 10 minutes and resuspended in 0.05 M sodium phosphate, 0.3 M sodium chloride, 0.02 M imidazole (pH 8.0) buffer containing lysozyme and DNaseI. Resuspended cells were lysed by repeated passes through a French Pressure cell at 20,000 psi. Cell lysates were clarified by ultracentrifugation at 229,000×g for one hour. The supernatants were loaded onto a HiTrap IMAC HP column charged with nickel sulfate and equilibrated with 0.05 M sodium phosphate, 0.3 M sodium chloride, 0.02 M imidazole (pH 8.0). Enzymes were isolated using a linear gradient from 0.02 to 0.5 M of imidazole. Fractions containing the protein of interest were identified using SDS-PAGE (Invitrogen), pooled and desalted into 0.05 M HEPES, 0.05 M sodium chloride (pH 7.4) with 1 mM DTT using a Hi Prep 26/10 desalting column. Both MvaS and MvaE were further purified over an anion exchange HiTrap Q HP column. The column was washed with 0.05 M Tris, 0.05 M sodium chloride (pH 7.6) with 1 mM DTT and eluted with a 0.05-1.0 M sodium chloride gradient. E. faecalis MvaE was further purified using Superdex 200 10/300GL column which was equilibrated and eluted with 20 mM HEPES, 150 mM sodium chloride pH (7.4). The purity of both enzymes was greater than 95% as judged by SDS-PAGE and coomassie staining. The proteins were optically quantitated at 280 nm using the following conversion factors: 0.898 OD/mg/mL for E. faecalis MvaS and 0.49 OD/mg/mL for E. faecalis MvaE. These values were obtained using the ExPASy ProtParam tool.

Expression and Purification of Recombinant IDI from S. Cerevisiae and IspS from P. Alba

E. coli cells expressing his tagged IDI and his tagged IspS (SEQ ID NO:5 and 6, respectively) were grown in Luria-Bertani broth supplemented with 50 μg/mL carbenicillin and 30 μg/mL chrolamphenicol. Cells harboring IDI were induced overnight with the addition of 0.2 mM IPTG at an OD600 of ˜0.4-0.6, whereas cells harboring the ispS expression plasmid were induced with 0.4 mM IPTG at an OD of 0.4 for 6 hours. Cells were harvested by centrifugation at 10,000×g for 10 minutes and resuspended in 0.05 M sodium phosphate, 0.3 M sodium chloride, 0.02 M imidazole (pH 8.0) buffer containing lysozyme and DNaseI. Resuspended cells were lysed by repeated passes through a French Pressure cell at 20,000 psi. Cell lysates were clarified by ultracentrifugation at 229,000×g for one hour. The supernatants were loaded onto a HiTrap IMAC HP column charged with nickel sulfate and equilibrated with 0.05 M sodium phosphate, 0.3 M sodium chloride, 0.02 M imidazole (pH 8.0). Enzymes were isolated using a linear gradient from 0.02 to 0.5 M of imidazole. Fractions containing the protein of interest were identified using SDS-PAGE (Invitrogen), pooled and desalted into 0.05 M HEPES, 0.05 M sodium chloride (pH 7.4) with 1 mM DTT using a Hi Prep 26/10 desalting column. The purity of isolated enzymes was greater than 95% as judged by SDS-PAGE and coomassie staining. The proteins were optically quantitated at 280 nm using a conversion factor of 1.333 OD/mg/mL for IDI and 1.35 OD/mg/mL for ispS(these values were obtained using the ExPASy ProtParam tool).

### Polyclonal Antibody Production

One milligram of each purified protein was sent to ProSci™ (Poway, Calif.) for polyclonal antibody production in rabbits. Second antisera bleeds were screened via western blots and used for protein detection.

### Example 1

### Production of Isoprene from Syngas Derived from Various Sources without Oxygen

The yield of isoprene from syngas depends upon the composition of the syngas. The generalized stoichiometric equation for the conversion of syngas to isoprene is shown in Equation 7.

(5-n)CO₂+nCO+(14-n)H₂+(n-10)H₂O→1Isoprene+(n-5)CO₂+(10-n)H₂O Equation 7

The moles of CO₂, CO, H₂, and H₂O in the syngas and the resulting stoichiometric molar yields of isoprene, CO₂, and H₂O according to Equation 7 are shown in Table 6 for n of 0 through 14. The same values are depicted graphically in FIG. 2. Negative values generated from the equation are treated as zero. Values of zero indicate that a component of the equation is absent from the syngas or is not produced as a product of the reaction. For example, in some aspects the syngas lacks one or more of CO₂, CO, H₂, and H₂O. In some aspects, CO₂ or H₂O are not produced by the reaction.

TABLE 6

| Moles | Moles | Moles | Moles | | Moles | Moles | Moles |
|---|---|---|---|---|---|---|---|
| of CO₂ | of CO | of H₂ | of H₂O | | of C₅H₈ | of CO₂ | of H₂O |
| react- | react- | react- | react- | | pro- | pro- | pro- |
| ing | ing | ing | ing | — | duced | duced | duced |
| 0 | 14 | 0 | 4 | | 1 | 9 | 0 |
| 0 | 13 | 1 | 3 | | 1 | 8 | 0 |
| 0 | 12 | 2 | 2 | | 1 | 7 | 0 |
| 0 | 11 | 3 | 1 | | 1 | 6 | 0 |
| 0 | 10 | 4 | 0 | | 1 | 5 | 0 |
| 0 | 9 | 5 | 0 | | 1 | 4 | 1 |
| 0 | 8 | 6 | 0 | | 1 | 3 | 2 |
| 0 | 7 | 7 | 0 | | 1 | 2 | 3 |
| 0 | 6 | 8 | 0 | | 1 | 1 | 4 |
| 0 | 5 | 9 | 0 | | 1 | 0 | 5 |
| 1 | 4 | 10 | 0 | | 1 | 0 | 6 |
| 2 | 3 | 11 | 0 | | 1 | 0 | 7 |
| 3 | 2 | 12 | 0 | | 1 | 0 | 8 |
| 4 | 1 | 13 | 0 | | 1 | 0 | 9 |
| 5 | 0 | 14 | 0 | | 1 | 0 | 10 |

I. Production of Isoprene from Syngas Derived from Biomass

Isoprene is produced under anaerobic conditions from syngas derived from biomass. The biomass is consistent with reported molecular compositions of syngas produced from biomass. The approximate stoichiometric composition of biomass is CH_{1.4}O_{0.6}. (see “Reed, T. The Fuel Composition-Conversion Diagram. The Biomass Energy Foundation)

Conversion of biomass to syngas proceeds according to Equation 8.

CH_{1.4}O_{0.6}+0.4H₂O→CO+1.1H₂ Equation 8

Accordingly, the maximum theoretical isoprene mass yield from biomass (not including water)=44.4%.

The production of isoprene from syngas produced from biomass results in a greater theoretical yield than production of isoprene from carbohydrates that are metabolized through glycolytic pathways by microorganisms. The maximum yield of isoprene from carbohydrates is 32.4%.

II. Production of Isoprene from Syngas Derived from Coal

Isoprene is produced under anaerobic conditions from syngas derived from coal. The coal is consistent with reported molecular compositions of syngas produced from coal. The approximate stoichiometric composition of coal is CH. (see “Reed, T. The Fuel Composition-Conversion Diagram. The Biomass Energy Foundation)

Conversion of coal to syngas proceeds according to Equation 9.

CH+H₂O→CO+1.5H₂ Equation 9

Accordingly, the maximum theoretical isoprene mass yield from coal (not including water)=93.6%.

The production of isoprene from syngas produced from coal results in a greater theoretical yield than production of isoprene from carbohydrates that are metabolized through glycolytic pathways by microorganisms. The maximum yield of isoprene from carbohydrates is 32.4%.

III. Production of Isoprene from Syngas Derived from Rubber Tires

Isoprene is produced under anaerobic conditions from syngas derived from rubber tires. The rubber tires are consistent with reported molecular compositions of syngas produced from rubber tires. The approximate stoichiometric composition of rubber tires is CH_{1.6}. (see “Reed, T. The Fuel Composition-Conversion Diagram. The Biomass Energy Foundation)

Conversion of rubber tires to syngas proceeds according to Equation 10.

CH_{1.6}+H₂O→CO+1.8H₂ Equation 10

Accordingly, the maximum theoretical isoprene mass yield from rubber tires (not including water)=100%.

The production of isoprene from syngas produced from rubber tires results in a greater theoretical yield than production of isoprene from carbohydrates that are metabolized through glycolytic pathways by microorganisms. The maximum yield of isoprene from carbohydrates is 32.4%.

IV. Production of Isoprene from Syngas Derived from Municipal Solid Waste

Isoprene is produced under anaerobic conditions from syngas derived from municipal solid waste. The municipal solid waste is consistent with reported molecular compositions of syngas produced from municipal solid waste. The approximate stoichiometric composition of municipal solid waste is CH_{2.3}O_{0.6}. (see “Reed, T. The Fuel Composition-Conversion Diagram. The Biomass Energy Foundation)

Conversion of municipal solid waste to syngas proceeds according to Equation 11.

CH_{2.3}O_{0.6}+0.4H₂O→CO+1.55H₂ Equation 11

Accordingly, the maximum theoretical isoprene mass yield from municipal solid waste (not including water)=51.9%.

The production of isoprene from syngas produced from municipal solid waste results in a greater theoretical yield than production of isoprene from carbohydrates that are metabolized through glycolytic pathways by microorganisms. The maximum yield of isoprene from carbohydrates is 32.4%.

### Example 2

### Production of Fuels from Isoprene Generated from Syngas

Isoprene derived from synthesis gas is converted to compounds with value as fuels. For example isoprene is hydrotreated with hydrogen in the presence of a catalyst to give monounsaturated isoamylenes and/or isopentane. These compounds can be blended directly into gasoline, or further processed into higher hydrocarbons in the C6 to C20 range by means of chemical catalysis methods known to those skilled in the art such as olefin dimerization and isoparaffin alkylation.

Alternately, isoprene is first oligomerized to C10 dimers and/or C15 trimers, both cyclic and linear using metal-based-catalysts, for example those based upon nickel, chromium, iron, ruthenium and palladium metals (other exemplary catalysts can be found in U.S. Provisional Patent Application Ser. No. 61/187,944, filed on Jun. 17, 2009) followed by hydrotreating to saturate double bonds. Isoprene is also dimerized to C10 compounds when heated to 150 to 250° C. in the presence of an antioxidant (see for example U.S. Pat. No. 4,973,787). The resulting C10 and C15 compounds are useful in gasoline and diesel blends.

### Example 3

### Production of Microbial Fuels from Syngas

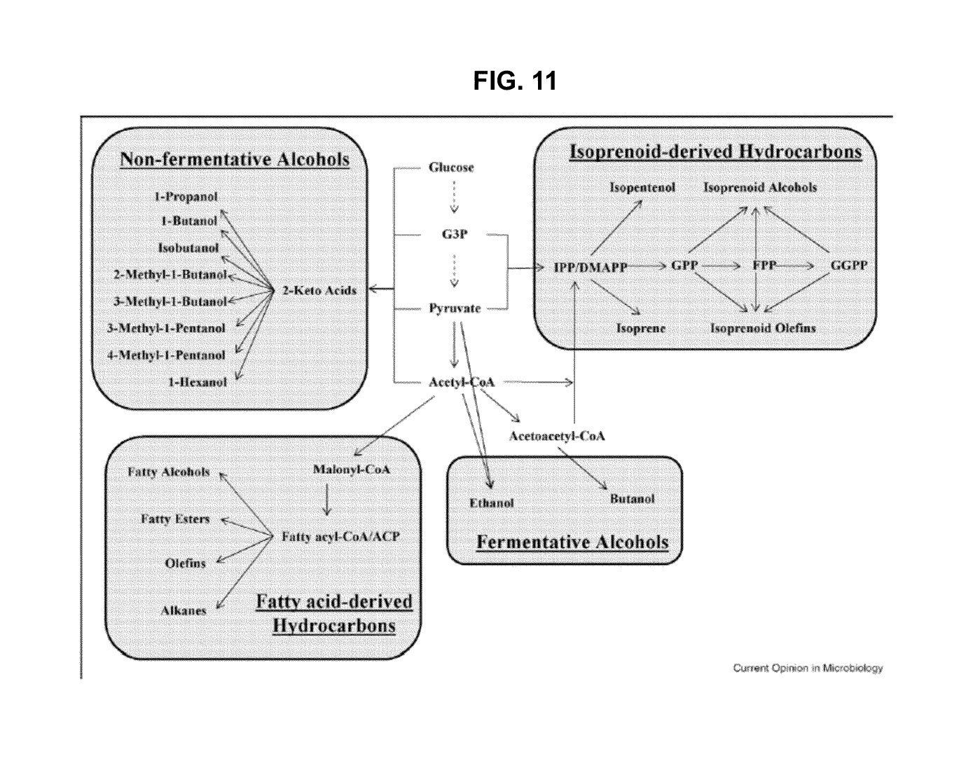

This example demonstrates increased production of fermentative alcohols, e.g., ethanol, butanol; non-fermentative alcohols, e.g., isobutanol, methyl butanol; fatty alcohols, esters; isoprenoid alcohols, alkenes. Most of these compounds are synthesized from engineered pathway utilizing building block AcCoA via syngas fermentation. Pathways for production of these products are illustrated in FIG. 11.

Volatile fermentation products can be produced and recovered from synthesis gas fermentations, in addition to isoprene. Such compounds can be recovered from the fermentation off-gas stream provided that their volatility is high enough to prevent accumulation to toxic levels in the fermentor. Examples include, but are not limited to methanol, acetone, acetaldehyde, diacetyl, methyl acetate, ethyl acetate, diethyl ether and C2 to C4 hydrocarbons.

### Example 4

### Production of Isoprene from Syngas Produced by Water Reforming Reactions

Syngas is produced from a variety of feedstocks by water reforming reactions. The molar ratio of carbon monoxide to hydrogen in the syngas depends on the feedstock used. Feedstock compositions of sugar, biomass, coal rubber tires, and municipal solid waste and the resulting syngas compositions after water reforming are shown in Table 7. Anaerobic cells containing a heterologous nucleic acid encoding isoprene synthase are cultured in the presence of each of the syngas compositions shown in Table 7. The cells produce isoprene in mass yields up to the maximum mass yields provided in Table 7.

TABLE 7

| | | | Maximum |
|---|---|---|---|
| Feedstock | C, H, and O | CO and H₂ | Isoprene |
| Compositions | Compositions | Compositions | Yield |
| Sugar | C—H₂—O + 0 H₂O | CO + 1 H₂ | 32.4% |
| Biomass | C—H_{1.4}—O_{0.6} + | CO + 1.1 H₂ | 44.4% |
| | 0.4 H₂O | | |
| Coal | C—H + H₂O | CO + 1.5 H₂ | 93.6% |
| Rubber Tires | C—H_{1.6} + H₂O | CO + 1.8 H₂ | 100.2% |
| Municipal Solid | C—H_{2.3}—O_{0.6} + | CO + 1.55 H₂ | 51.9% |
| Waste | 0.4 H₂O | | |

### Example 5

### Production of Isoprene from Syngas Produced by Oxygen Reforming Reactions

Syngas is produced from a variety of feedstocks by oxygen reforming reactions. The molar ratio of carbon monoxide to hydrogen in the syngas depends on the feedstock used. Feedstock compositions of sugar, biomass, coal rubber tires, and municipal solid waste and the resulting syngas compositions after oxygen reforming are shown in Table 8. Anaerobic cells containing a heterologous nucleic acid encoding isoprene synthase are cultured in the presence of each of the syngas compositions shown in Table 8. The cells produce isoprene in mass yields up to the maximum mass yields provided in Table 8.

TABLE 8

| | | | Maximum |
|---|---|---|---|
| Feedstock | C, H, and O | CO and H₂ | Isoprene |
| Compositions | Compositions | Compositions | Yield |
| Sugar | C—H₂—O + 0.1 O₂ | CO + 0.8 H₂ | 29.2% |
| Biomass | C—H_{1.4}—O_{0.6} + 0.2 O₂ | CO + 0.7 H₂ | 36.0% |
| Coal | C—H + 0.5 O₂ | CO + 0.5 H₂ | 56.1% |
| Rubber Tires | C—H_{1.6} + 0.5 O₂ | CO + 0.8 H₂ | 64.4% |
| Municipal Solid | C—H_{2.3}—O_{0.6} + 0.2 O₂ | CO + 1.15 H₂ | 43.8% |
| Waste | | | |

### Example 6

### Production of Isoprene from Syngas Produced by Oxygen and Water Reforming Reactions

Syngas is produced from a variety of feedstocks by oxygen and water reforming reactions. The molar ratio of carbon monoxide to hydrogen in the syngas depends on the feedstock used. Feedstock compositions of sugar, biomass, coal rubber tires, and municipal solid waste and the resulting syngas compositions after oxygen and water reforming are shown in Table 9. Anaerobic cells containing a heterologous nucleic acid encoding isoprene synthase are cultured in the presence of each of the syngas compositions shown in Table 9. The cells produce isoprene in mass yields up to the maximum mass yields provided in Table 9.

TABLE 9

| | | | Maximum |
|---|---|---|---|
| Feedstock | | CO and H₂ | Isoprene |
| Compositions | C, H, and O Compositions | Compositions | Yield |
| Sugar | C—H₂—O + 0 H₂O + 0.1 O₂ | CO + 0.8 H₂ | 29.2% |
| Biomass | C—H_{1.4}—O_{0.6} + 0.2 H₂O + | CO + 0.9 H₂ | 40.2% |
| | 0.1 O₂ | | |
| Coal | C—H + 0.5 H₂O + 0.25 O₂ | CO + 1 H₂ | 74.9% |
| Rubber Tires | C—H_{1.6} + 0.5 H₂O + 0.25 O₂ | CO + 1.3 H₂ | 82.3% |
| Municipal | C—H_{2.3}—O_{0.6} + 0.2 H₂O + | CO + 1.35 H₂ | 47.8% |
| Solid | 0.1 O₂ | | |
| Waste | | | |

### Example 7

### Removal of Impurities from Synthesis Gas Feeds

Synthesis gas contains numerous impurities, the nature and amount of which vary according to both the feedstock and the process used in production. In general, fermentations are more tolerant to many of these impurities than some of the catalysts used in GTL (gas to liquid) technologies, such as those based upon Fischer-Tropsch chemistry. There remains the need to remove from the syngas materials that might foul the fermentor and associated equipment such as tars and particulates. It is also advisable to removal of compounds that might contaminate the isoprene product such as volatile organic compounds, methane, benzene, toluene, ethylbenzene, xylene, H₂S, COS, CS₂, HCl, O₃, organosulfur compounds and metals.

Removal of impurities from syngas is achieved by one of several means including gas scrubbing, treatment with solid-phase adsorbents and purification using gas-permeable membranes.

### Example 8

### Analysis of Impurities in Isoprene Recovered from Syngas Fermentations

Isoprene recovered from synthesis gas fermentations is analyzed by GC/MS, GC/FID and ¹H NMR to determine the identity and levels of impurities. Impurities are characterized as to whether they are of microbial origin, or as contaminants in the synthesis gas feed or other fermentation raw materials. Impurities include, but are not limited to hydrogen sulfide, carbonyl sulfide, carbon disulfide, ethanol, acetone, methanol, acetaldehyde, methacrolein, methyl vinyl ketone, 2-methyl-2-vinyloxirane, cis- and trans-3-methyl-1,3-pentadiene, a C5 prenyl alcohol (such as 3-methyl-3-buten-1-ol or 3-methyl-2-buten-1-ol), 2-heptanone, 6-methyl-5-hepten-2-one, 2,4,5-trimethylpyridine, 2,3,5-trimethylpyrazine, citronellal, methanethiol, ethanethiol, methyl acetate, 1-propanol, diacetyl, 2-butanone, 2-methyl-3-buten-2-ol, ethyl acetate, 2-methyl-1-propanol, 3-methyl-1-butanal, 3-methyl-2-butanone, 1-butanol, 2-pentanone, 3-methyl-1-butanol, ethyl isobutyrate, 3-methyl-2-butenal, butyl acetate, 3-methylbutyl acetate, 3-methyl-3-buten-1-yl acetate, 3-methyl-2-buten-1-yl acetate, (E)-3,7-dimethyl-1,3,6-octatriene, (Z)-3,7-dimethyl-1,3,6-octatriene, (E,E)-3,7,11-trimethyl-1,3,6,10-dodecatetraene and (E)-7,11-dimethyl-3-methylene-1,6,10-dodecatriene, 3-hexen-1-ol, 3-hexen-1-yl acetate, limonene, geraniol (trans-3,7-dimethyl-2,6-octadien-1-ol), citronellol (3,7-dimethyl-6-octen-1-ol), (E)-3-methyl-1,3-pentadiene and (Z)-3-methyl-1,3-pentadiene, thiol(s), mono and/or disulfide(s), and/or gas(es) such as CS₂ and/or COS.

### Example 9

### Expression of MvaE and MvaS Polypeptides in Clostridium ljungdahlii Grown on Fructose

FIG. 4 shows a schematic representation of an obligate anaerobe engineered with mvaE and mvaS to express the upper MVA pathway.

### Preparation of Electrocompetent C. Ljungdahlii

Five mL of MES-F medium (see Tables 10 and 11) was inoculated with 0.1 mL of a frozen stock of wild type (WT) Clostridium ljungdahlii (ATCC 55383) cells frozen in 1:1 mixture of MES-F medium+50% glycerol in a Coy (Grass Lake, Mich.) Type B anaerobic chamber (atmosphere 2% H₂, 5% CO₂, 91% N₂) and cultured on an incubator shaker at 37° C. The cells were repassaged after 24 h on fresh medium and then repassaged again after another 24 h. Two bottles containing 100 mL each of MES-F medium containing 40 mM DL-threonine were inoculated with the repassaged cells at a concentration of 0.004 OD₆₀₀ and allowed to grow up until the cell density reached 0.1 OD₆₀₀. The 200 mL of cells were harvested by centrifuging in a RC5B preparative centrifuge, (Sorvall Instruments, Wilmington, Del.) for 10 min at 5000 rpm outside the chamber in four 50 mL deoxygenated sealed centrifuge tubes (stored in anaerobic chamber) using a HS-4 Sorvall Instruments (Wilmington, Del.) swinging bucket rotor at <10° C. The tubes were returned to the anaerobic chamber, the supernatant poured off and the pellets resuspended in two 50 mL volumes of ice-cold deoxygenated SMP buffer (270 mM sucrose, 7 mM Na(H)₂PO₄, 1 mM MgSO₄, pH5.9). The suspension was again centrifuged outside the chamber in two 50 mL deoxygenated sealed tubes under the same conditions in the precooled rotor. The pellets were again washed with ice-cold SMP buffer in two 50 mL tubes. The pellets were then combined and resuspended one last time in 50 mL ice cold deoxygenated SMP buffer. After centrifuging again as above, the pellet was resuspended in the anaerobic chamber in a small volume of ice-cold SMP buffer (typically ˜400 μL) to which was added a 20% volume of 60% DMSO 40% SMP buffer. The suspension on ice was aliquoted out in 25 μL volumes into individual cryotubes previously incubated in the anaerobic chamber and quick frozen using liquid N₂. The frozen cells were stored in a liquid N₂ dewar.

TABLE 10
MES-F Growth Medium Composition and Final Concentration

| | | | stock | vol. | | lx MES-F |
|---|---|---|---|---|---|---|
| Media | | stock | molarity | stock or | | final |
| Component | f. wt | g/L | (M) | g/liter | | (mM) |
| NH4Cl | 53.4 g | 100 | 1.87 | 10 | ml | 18.7 |
| KH2PO4 | 136.09 | 100 | 0.73 | 2 | ml | 1.46 |
| MgSO4.7H20 | 246.47 | 100 | 0.406 | 2 | ml | 0.811 |
| KCl | 74.55 | 100 | 1.34 | 1 | ml | 1.34 |
| CaCl2.2H2O | 147.01 | 20 | 0.136 | 1 | ml | 0.136 |
| Sodium | 136.08 | 166 | 1.22 | 2.5 | ml | 3.05 |
| Acetate | | | | | | |
| Cysteine | 175.6 | | | 0.879 | g | 5.01 |
| HCl | | | | | | |
| Wolfe's | | | | 10 | ml | |
| vitamin | | | | | | |
| solution | | | | | | |
| Ljungdahlii | | | | 10 | ml | |
| trace metals | | | | | | |
| Resazurin | 229.19 | 1 | 0.00436 | 1 | ml | 4.36 × 10⁻³ |
| Yeast | | | | 2 | g | |
| Extract | | | | | | |
| MES | 195.2 | | | 20 | g | 102.45 |
| Fructose | 180.16 | | | 10 | g | 55.5 |

TABLE 11
Components of Trace Metals Mix
Used in C. ljungdahlii MES-F Growth Media

| Component | Amount | |
|---|---|---|
| Nitrilotriacetic acid | 2.0 | g |
| MnSO₄ · H₂O | 1.0 | g |
| Fe(SO₄)₂(NH₄)₂ · 6H₂O | 0.8 | g |
| CoCl₂ · 6H₂O | 0.2 | g |
| ZnSO₄ · 7H₂O | 0.2 | mg |
| CuCl₂ · 2H₂O | 20.0 | mg |
| NiCl₂ · 6H₂O | 20.0 | mg |
| Na₂MoO₄ · 2H₂O | 20.0 | mg |
| Na₂SeO₄ | 20.0 | mg |
| Na₂WO₄ | 20.0 | mg |
| Distilled water | 1.0 | L |

When making the trace metals mix for the MES-F growth media, nitrilotriacetic acid was added to water and adjusted to pH 6.0 with potassium hydroxide (KOH), then the remainder of the trace metals was added to complete the trace metals solution.

### Plasmid Construction of pJF 100

Heap et al. (Journal of Microbiological Methods 78 (2009) p79-85) describe construction of a series of modular E. coli-Clostridia shuttle vectors, the pMTL80000 series. The vectors carry one of four Gram positive replicons, a p15A or ColE 1 origin of replication in E. coli, a multiple cloning site with flanking transcriptional terminators and an antibiotic resistant marker: catP, ermB, aad9 or tetA. Some of the vectors also carry a C. sporogenes ferredoxin promoter (Pfdx) and ribosome binding site (RBS) or a C. acetobutylicum thiolase promoter and RBS for gene expression.

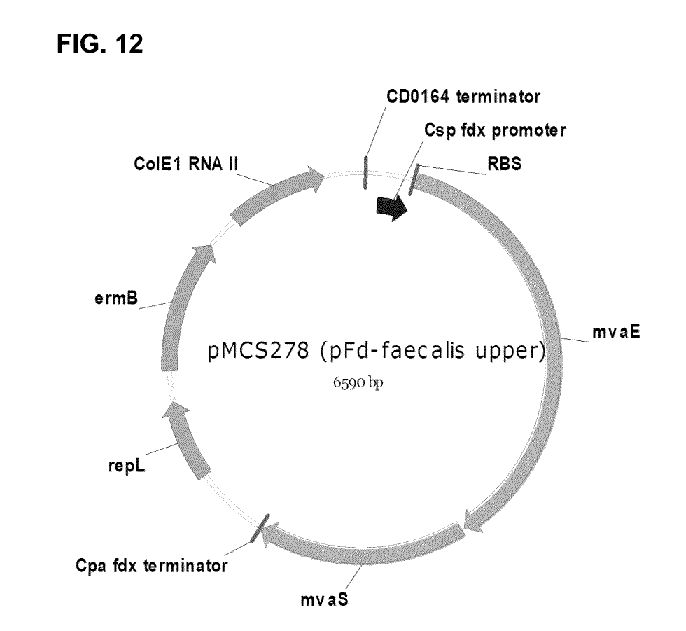

The mvaE coding region (SEQ ID NO:7), encoding acetyl-CoA acetyltransferase or 3-hydroxy-3-methylglutaryl-CoA (HMG-CoA) reductase SEQ ID NO:8), and mvaS coding region (SEQ ID NO:9), encoding HMG-CoA synthase (SEQ ID NO:10), both from Enterococcus faecalis were cloned as an operon under the control of the Pfdx promoter, with the C. sporogenes ferredoxin terminator (Cpa fdx terminator), in a modular vector with a pIM 13 Gram positive replicon, the ColE1 origin of replication for E. coli, and the ermB marker creating pMCS278 (SEQ ID NO:11; plasmid map in FIG. 12 (repL is the pIM13 replicon).

The vector pMTL83151, renamed pMCS201 (SEQ ID NO:12), carries the pCB102 Gram positive origin of replication (SEQ ID NO: 1; isolated from a plasmid of C. butyricum) the catP marker, and the ColE1 E. coli origin of replication. Transformation of C. ljungdahlii was successful under the conditions described here using either pCB102 or pBP1. The origin of replication in pBP1 (SEQ ID NO:62) was isolated from a plasmid of C. botulinum.

The vector pMCS278 was digested with restriction enzymes PmeI and AscI (New England Biolabs, Inc). The 4.8 kb vector fragment was gel purified with the QIAquick Gel Extraction Kit (Qiagen Inc). The digest removes the ermB marker and Gram positive replicon pIM13 from the vector. Plasmid pMCS201 was digested with PmeI, AscI and ApaI (New England Biolabs, Inc) and the 2.4 kb insert containing the catP marker and pCB 102 replicon was gel purified with the QIAquick Gel Extraction Kit (Qiagen Inc). The vector and insert were ligated with T4 DNA ligase (New England Biolabs, Inc) according to manufacturer's protocol at room temperature overnight. The ligation was transformed into chemically competent E. coli Top10 cells (Invitrogen) following manufacturer's instructions. After outgrowth in SOC (Invitrogen, Carsbad, Calif., Part No. 46-0700), aliquots of the transformation mix were plated onto Luria Broth (LB) plates with 15 μg/mL of chloramphenicol. Plates were incubated overnight at 30° C.

Transformants were screened by colony PCR with HotStarTaq Master Mix (Qiagen) with primers Upper F4 (SEQ ID NO:13) and Upper R2 (SEQ ID NO:14) for an approximate 640 bp PCR product. PCR amplification conditions were: 94° C., 15 min; 94° C. (0.5 min)-55° C. (0.5 min)-72° C. (1 min) for 30 cycles, and 72° C. for 10 min. Several colonies that amplified the correct sized product were sequenced by Templiphi (GE Health Care Life Sciences) using primers UpperF1 (SEQ ID NO:15), UpperR6 (SEQ ID NO:16), UpperF4 (SEQ ID NO:13), UpperR2 (SEQ ID NO:14), and CatP-F (SEQ ID NO:17).

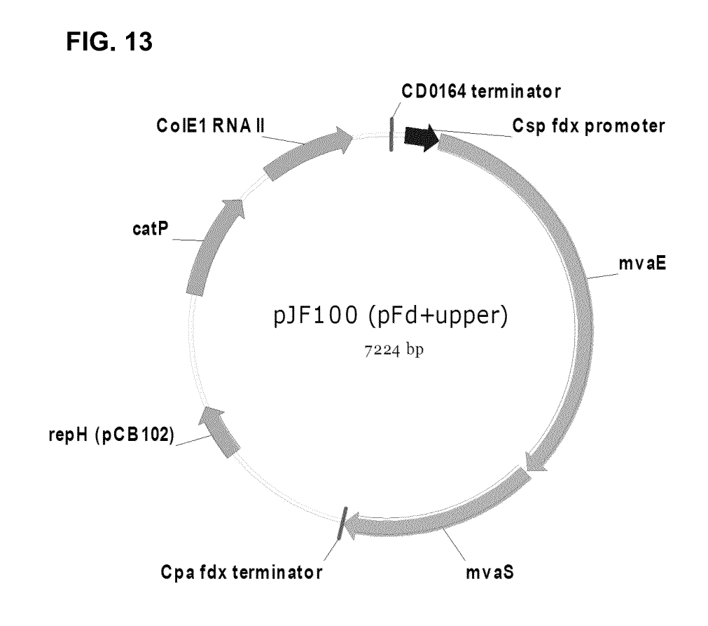

After confirmation of the correct sequence, a transformant was grown at 30° C. in LB medium containing 15 μg/mL of chloramphenicol with shaking at 220 rpm. After overnight growth the culture was centrifuged at 5,000 g for 10 minutes and the supernatant decanted. Plasmid DNA was isolated from the pelleted culture using a QIAprep Spin Miniprep Kit (Qiagen) following the manufacturer's instructions. The resulting plasmid was named pJF 100 (SEQ ID NO:18). The plasmid map of pJF100 is shown in FIG. 13.

### Electroporation of Plasmids

A cryotube containing frozen electrocompetent C. ljungdahlii cells was thawed in the anaerobic chamber by placing on water ice. Two micrograms of plasmid DNA at ˜1 μg/μL were added and mixed with the cells. The suspension was then placed in a prechilled (on ice) 1 mm gap electroporation cuvette (Gene Pulser cuvette, Bio-Rad, Hercules, Calif.), previously incubated in the anaerobic chamber, and electroporated (Gene Pulser Xcell, Bio-Rad, Hercules, Calif.) at 625 V, with a resistance of 600Ω and a capacitance of 25 μF. Five hundred microliters of pre-chilled MES-F medium, previously deoxygenated by incubation in the anaerobic chamber, were added to the cuvette and the contents transferred to a vial containing 10 mL MES-F. The vial was sealed and incubated overnight on an incubator shaker at 37° C. at 110 rpm (Incu-Shaker Mini Shaking Incubator; Chemglass Life Sciences, Vineland, N.J.). The cell suspension was spun down in the anaerobic chamber using a VWR Clinical 200 Centrifuge (VWR International, Inc, West Chester, Pa.) at 6000 rpm for 15 min. The pellet was resuspended in 200 μl of MES-F medium. One hundred microliters were then distributed using a spreader across a petri plate (1.5% agar) containing enriched MES-F medium (MES-F supplemented with 10 g/L proteose peptone, 10 g/L beef extract) containing 5 μg/mL thiamphenicol. The plates were transferred to an anaerobic jar (Oxoid HP0011A, Oxoid Limited, Basinstoke, U.K.) which was then sealed inside the anaerobic chamber. The Oxoid jar (containing palladium catalyst) was removed from the chamber and placed in an incubator at 37° C. Colonies observed after 3 days were restreaked in the anaerobic chamber on petri plates with MES-F medium both containing 5 μg/mL thiamphenicol. These were then returned to the Oxoid jar and again incubated at 37° C. outside the chamber.

Plasmid Recovery from Clostridium ljungdahlii

C. ljungdahlii transformants were streaked onto fresh MES-F plates containing 5 μg/mL thiamphenicol. Plates were placed in a sealed Oxoid jar (Oxoid HP0011A, Oxoid Limited, Basinstoke, U.K.) and incubated at 37° C. After incubation, transformants were screened by colony PCR with HotStarTaq Master Mix (Qiagen) with primers Upper F4 (SEQ ID NO:13) and Upper R2 (SEQ ID NO:14) for an approximate 640 bp PCR product. PCR amplification conditions were: 94° C., 15 min; 94° C. (0.5 min)-55° C. (0.5 min)-72° C. (1 min) for 30 cycles, and a final extension of 72° C. for 10 min.

Colonies producing the expected PCR product were inoculated into 10 mL of MES-F medium in a serum vial. The vial was incubated at 37° C. with shaking at 110 rpm in an Incu-Shaker Mini Shaking Incubator (Chemglass Life Sciences. Vineland N.J.). After overnight incubation, the optical cell density was measured in an Eppendorf Biophotometer Plus (Eppendorf North America, Hauppauge, N.Y.). After the cells reached an OD₆₀₀ of approximately 0.2, the cells were harvested by centrifugation at 6000 rpm for 15 minutes in a VWR Clinical 200 Centrifuge (VWR International, Inc, West Chester, Pa.) and the supernatant poured off. To each pellet 250 μl of Buffer P1 with RNAse A solution (Qiagen) with 50 mg/mL lysozyme (L6876, Sigma Life Science) was added and the resuspended cells were transferred to a 1.5 ml, Eppendorf Flex-Tube (Eppendorf North America, Hauppauge, N.Y.). All steps were performed in the anaerobic chamber until after the pellet was resuspended in the P1 lysis buffer. Once resuspended, the cells were removed from the anaerobic chamber and incubated at 37° C. for 1 hour. After the incubation, the lysed cells were processed for plasmid DNA using QIAprep Spin Miniprep Kit (Qiagen) following the Kit instructions. E. coli chemically competent Top10 cells (Invitrogen) were transformed with the resulting isolated DNA according to manufacturer's instructions. After outgrowth in SOC, cells were plated onto Luria Broth (LB) plates containing 15 μg/mL of chloramphenicol. Plates were incubated overnight at 30° C.

E. coli Top10 transformants were screened by colony PCR with HotStarTaq Master Mix (Qiagen) with primers Upper F4 and Upper R2 for an approximate 640 bp PCR product. PCR amplification conditions were: 94° C., 15 min; 94° C. (0.5 min)-55° C. (0.5 min)-72° C. (1 min) for 30 cycles, and 72° C. for 10 min.

Colonies producing the expected size PCR product were inoculated into Luria Broth containing 15 μ/mL chloramphenicol and incubated overnight with shaking at 220 rpm at 30°C. Plasmid was isolated from overnight cultures with QIAprep Spin Miniprep Kit (Qiagen) following the Kit instructions. Plasmid DNA was sequenced with an ABI DNA Sequencer 3730xl and BigDye Terminator Cycle Sequencing chemistry using the primers UpperR6 (SEQ ID NO:16), UpperF1 (SEQ ID NO:15), UpperF2 (SEQ ID NO:19), UpperF3 (SEQ ID NO:20), UpperF4 (SEQ ID NO:13), UpperF5 (SEQ ID NO:21), and UpperF6 (SEQ ID NO:22). The expected sequence was obtained.

### Western Blotting to Detect Expression of MvaE and MvaS

C. ljungdahlii WT (ATCC 55383) and pJF100 transformants #2, #4, #6, and #8 were grown in 160 mL sealed bottles on MES-F medium+5 μg/mL thiamphenicol. All were inoculated from frozen stocks and incubated at 37° C. in an incubator shaker in the anaerobic chamber. At harvest, the cell densities (OD₆₀₀) were 0.864, 0.912, 0.634, 0.854 and 0.882, respectively. The cell suspensions were poured into 50 mL conical centrifuge tubes, which were sealed, removed from the anaerobic chamber and spun at 5000 rpm for 10 min at 5° C. in a HS-4 Sorvall rotor. The tubes were returned to the anaerobic chamber, the supernatants poured off and the pellets resuspended in 20 mL each of PBS (Phosphate buffered saline: 137 mM NaCl, 2.7 mM KCl, 10 mM Na₂HPO₄, 2.0 mM KH₂PO₄, pH 7.4). The tubes were again removed from the anaerobic chamber and spun as before in the HS-4 rotor. The tubes were returned to the anaerobic chamber and the supernatants poured off. The tubes were removed from the anaerobic chamber, frozen in liquid N₂ and stored in a −80° C. freezer.

In preparation for Western blotting, the pellets were thawed and resuspended in 1.5 mL PBS containing 100 μg/mL PMSF. The cell suspensions were passaged three times through a French pressure cell at 20,000 psi at ˜5° C. A portion of the lysates were used immediately for the Western blotting and the remainder aliquotted out, quick frozen in liquid N₂ and stored in a −80° C. freezer. Fifteen μL of lysate was mixed with 5 μL of LDS sample buffer (Invitrogen, Carlsbad Calif., Cat. No. NP0008) for each lane of each of three gels: two Western blots and one Simple Blue SafeStain-stained gel.

Expression of MvaE and MvaS in C. ljungdahlii was compared to that in E. coli TV3007 (also called MD12-778) transformed with the same pJF100 plasmid. E. coli TV3007 was previously shown to express the component genes of the mevalonate pathway and was used as a control. TV3007 was derived from E. coli strain MD-780 by curing of plasmid pRed-Et. as described in US Patent Application Publications 2013/0273625 and US 2013/0089906, which are incorporated herein by reference.

### Preparation of Electrocompetent TV3007 Cells

Low salt LB broth was inoculated from a seed culture grown up in Low salt LB broth. After 2-3 h growth at 37° C. (OD₆₀₀=0.5-0.6), the culture was transferred to a 50 mL conical tube and centrifuged for 15 min at 5000 rpm (Sorvall HS-4) at 4° C. The supernatant was discarded and the pellet resuspended in 25 mL ice-cold, sterile water. The suspension was again spun down as above and the supernatant discarded. The pellet was resuspended in 10 mL ice-cold sterile water and again spun down as above. The supernatant was again discarded and the pellet was resuspended in 1 mL ice-cold sterile water. After spinning down as above the pellet was suspended in 100 μL of ice-cold water and used immediately for electroporation.

One hundred μL electrocompetent TV3007 cells were mixed with 1 μL miniprep pJF100 plasmid DNA (10-100 ng DNA/μL) and loaded into a 1 mm gap electroporation cuvette (BioRad, Hercules, Calif., Cat No. 165-2089) pre-chilled on ice. The cell suspension was electroporated using a BioRad Bio-Rad MicroPulser set at 25 μF, 1.85 kV, 200 ohms and transformed with plasmids pMCS201, pJF100#2, #6 and #8 isolated from the C. ljungdahlii transformants. After electroporation, 250 μL of SOC solution (Invitrogen Part No. 46-0700) was added and the suspension agitated in an Eppendorf tube for 60 min at 250 rpm, 37° C. One hundred μL were then spread on petri plates containing low salt LB medium (Teknova, Cat. No. L8600) plus 5 μg/mL chloramphenicol. The plates were allowed to grow up overnight at 30° C.

The resulting transformants were cultivated overnight in low salt LB medium (Teknova, Cat. No. L8600)+5 μg/mL chloramphenicol and harvested at an OD₆₀₀ of 3.0. One mL of culture was spun down in an Eppendorf centrifuge (Eppendorf, Hamburg, Germany, Centrifuge 5424) at 14,000 rpm for 15 min and resuspended in 0.5 mL of water. Fifteen μL of cell suspension was mixed with 5 μL of LDS sample buffer (Invitrogen, Carlsbad Calif., Cat. No. NP0008) for each lane of each of three gels (NuPage 12% Bis-Tris Gel, Invitrogen Cat No. NP0034, Carlsbad, Calif.)—two Western blots and one Simple Blue SafeStain-stained gel and heated at 100° C. for 10 min.

### Gel Electrophoresis, Staining and Blotting

The samples were loaded onto three 10-well Nupage 12% Bis-Tris Gels (Invitrogen Cat. No. NP0034 in the presence of MES SDS Running Buffer (Invitrogen Cat. No. NP0002) and run for ˜50 min at constant voltage (200 V). After electrophoresis, the gel to be stained was placed in 100 ml DI water (Thermo Scientific) and shaken for 5 min. The water was discarded and the wash was repeated once. The gel was then stained with 20 mL Simple Blue SafetStain with gentle shaking for 1 h. The stain buffer was discarded and replaced with 20 mL DI water to destain for 1-3 h.

The protein bands on the unstained gels were transferred to a nitrocellulose membrane using an iBlot Gel Transfer Device (Invitrogen Cat. No. IB1001) according to the manufacturer's instructions. The membrane was then placed in Blocking solution and incubated for 30 min with gentle shaking. The membrane was then washed 2× for 5 min with 20 mL DI water. The membrane was then incubated with 15 mL of primary antibody solution for 1 h and then decanted. The membrane was then washed 3× for 5 min each with 20 mL 16-fold diluted Wash buffer. The membrane was then incubated with 15 mL of secondary antibody solution for 30 min which was then decanted. The membrane was then washed 3× for 5 min each with 20 mL 16-fold diluted Wash buffer (Western Breeze, Invitrogen, Carlsbad, Calif.). The membrane was then rinsed 2× for 2 min with 20 mL DI water. The membrane was then blotted between paper towels.

The primary antibodies used for each of the two gels were Rabbit polyclonal antibodies made by ProSci™ (Poway, Calif.) as described in General Methods: anti MvaE (E. faecalis) and Rabbit anti MvaS (E. faecalis). The primary antibody solution was a 1:1000 dilution of antiserum in Blocking solution (Western Breeze, Invitrogen, Carlsbad, Calif.). The secondary antibody solution was 2 μg/mL Alexa Fluor 488 goat anti-rabbit IgG (H+L) (Part No A11008, Life technologies, Carlsbad, Calif.) in Blocking solution. The fluorescent bands were detected in a ProteinSimple instrument (Fluor Chem M) according to the manufacturer's instructions.

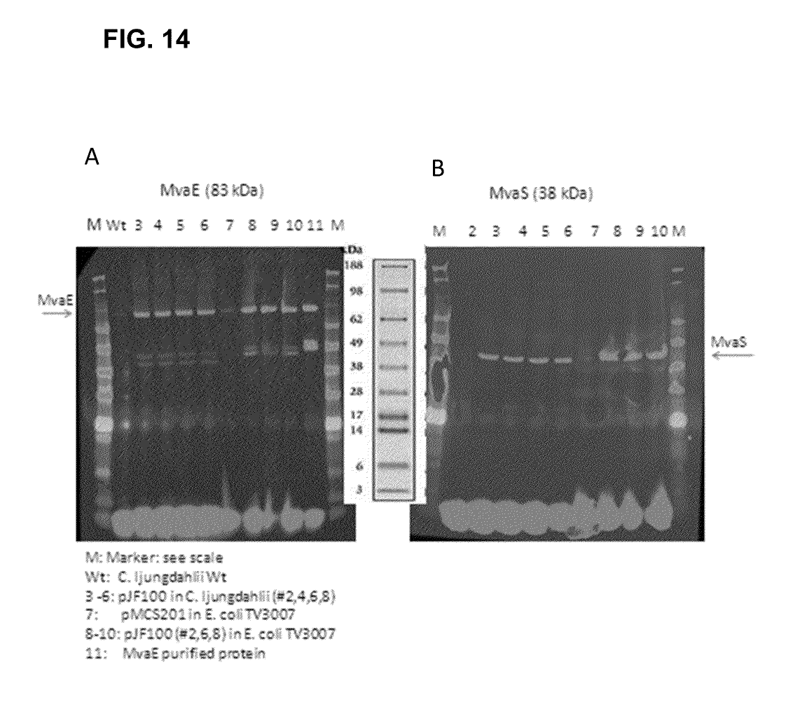

The Western Blots in FIG. 14 showed that the pJF 100 plasmid was functional in both E. coli TV3007 and in C. ljungdahlii and that mvaE and mvaS coding regions were expressed behind the C. sporogenes ferredoxin promoter. MvaE was partially proteolyzed in both C. ljungdahlii and E. coli TV3007, as indicated by doublet bands running between the 38 and 49 kDa markers. The parent band co-migrated with the MvaE marker at 83 kda. MvaS was stably expressed with a molecular weight of 38 kDa.

### Example 10

### Production of Mevalonate in Clostridium ljungdahlii Grown on Fructose

Growth of WT C. ljungdahlii, and pMCS201 and pJF100 C. ljungdahlii Transformants

Crimp-capped culture vials (total internal volume 14.5 mL) containing 10 mL of MES-F medium were inoculated from MES-F agar plates of WT C. ljungdahlii. The same crimp-capped culture vials with 10 mL of MES-F medium+5 μg/mL thiamphenicol were inoculated with cells of pMCS201- and pJF100-transformed C. ljungdahlii from enriched MES-F plates+5 μg/mL thiamphenicol. The culture vials were sealed with septa and crimp caps inside the anaerobic chamber. Crimp-capped bottles (160 mL total internal volume) containing 10 mL of the same medium as above were similarly inoculated with WT, pMCS201- and pJF100-transformed C. ljungdahlii. The septa of the 160 mL bottles were pierced with a 22-gauge sterile needle and capped by a sterile Super Acrodisc 13 (0.2 μm, Gelman Sciences, Ann Arbor, Mich., product no. 4602). All vials and bottles were placed on an incubator shaker at 37° C. and 110 rpm (Incu-Shaker Mini Shaking Incubator; Chemglass Life Sciences, Vineland, N.J.) inside the anaerobic chamber. The cultures were allowed to grow until they reached an OD₆₀₀ of 1-2. The OD₆₀₀s at harvesting for these cultures were 1.572 and 2.04 for WT sealed vial and equilibrated bottle, respectively; 1.80 and 1.904 for pMCS201 transformant sealed vial and equilibrated bottle, respectively; and 1.136 and 2.064 for pJF100 transformant sealed vial and equilibrated bottle, respectively. At this point 300 μL of culture was placed in 1.5 mL Eppendorf Flex-Tubes (Eppendorf North America, Hauppauge, N.Y.) to which were added 54 μl each of 10% H₂SO₄ according to the method of Keasling et al. (described in U.S. Pat. No. 7,183,089, p. 55) for HPLC determination of mevalonate. The acidification converts mevalonate to mevalonolactone. The samples were mixed and incubated for ≧45 min at 4° C. at which point they were loaded into HPLC vials with 200 μL inserts for injection into the HPLC.

### HPLC

A series 1100/1200 Agilent HPLC (Agilent Technologies, Wilmington, Del.) was used to analyze for mevalonolactone, which is produced spontaneously upon acidification of an aqueous solution of mevalonic acid. A Bio-Rad Aminex HPC-87H Ion Exclusion Column (300 mm×7.8 mm) fitted with a Micro-Guard Cation H Cartridge guard column (Cat. No. 125-0129, Bio-Rad, Hercules, Calif.) was equilibrated with a 0.01 NH₂SO₄ mobile phase at 60° C. at a flow rate of 0.6 mL/min. Sample volumes injected were 10 μL. Mevalonolactone was detected using an Agilent Refractive Index Detector (RID; G1362A) thermostatted at 55° C., in the elution profile between 18 and 21 min. Standard solutions of mevalonolactone in water at 0.25, 1.0, and 4.0 mg/mL were used as concentration standards to calibrate the measurements.

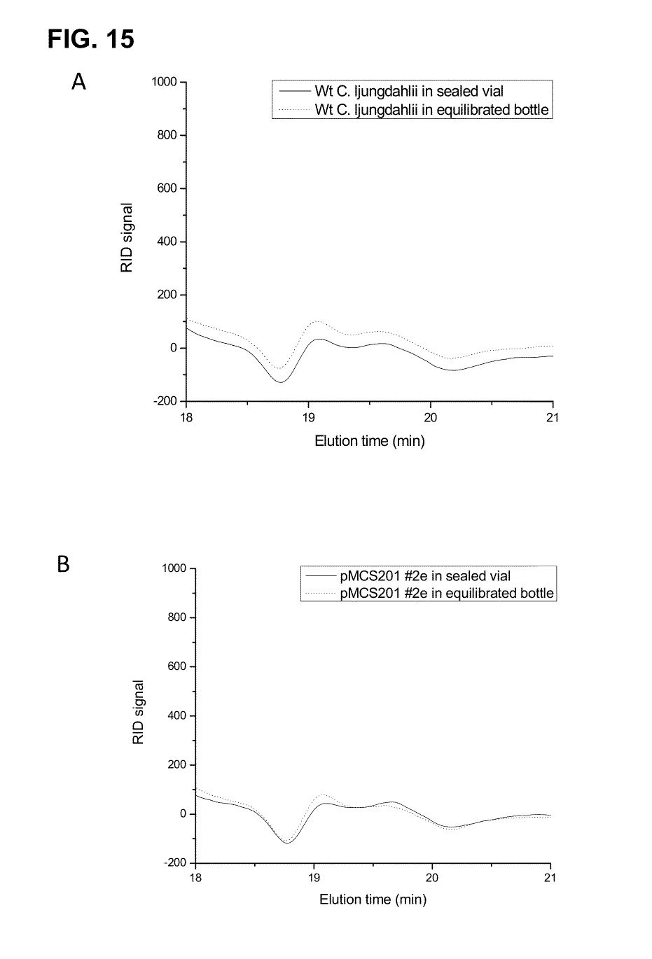

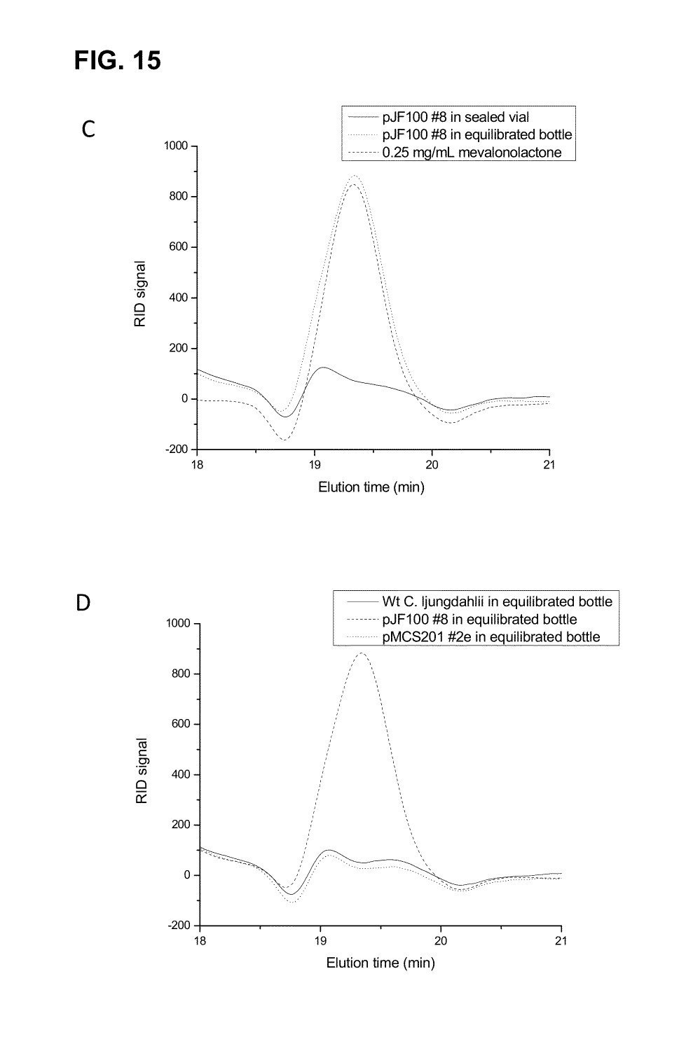

A comparison of the WT and the pMCS201 transformed C. ljungdahlii under sealed vial and chamber-equilibrated bottle growth conditions showed that there was little difference in the RID pattern for the two growth conditions for these two strains (FIGS. 15A and B), with no clear peak in the position of the mevalonate standard. In contrast, a comparison of the sealed vial and chamber-equilibrated bottle conditions for the pJF100 transformant showed that there was a large stimulation of mevalonate production (FIG. 15C) in the chamber-equilibrated bottle. FIG. 15C shows as well a comparison of the pJF100 transformant produced mevalonate (converted to mevalonolactone) with that of the 0.25 mg/mL mevalonolactone standard. After correction for dilution, the HPLC comparison indicated that, under the chamber-equilibrated conditions, there was approximately 0.3 mg/mL mevalonate in the culture medium for the pJF100 C. ljungdahlii transformant. FIG. 15D shows a comparison of the chamber-equilibrated conditions for all three strains with a mevalonate peak only in the case of the PJF 100 transformant.

Mevalonate Production from Fructose Under Chamber and N₂ Gas

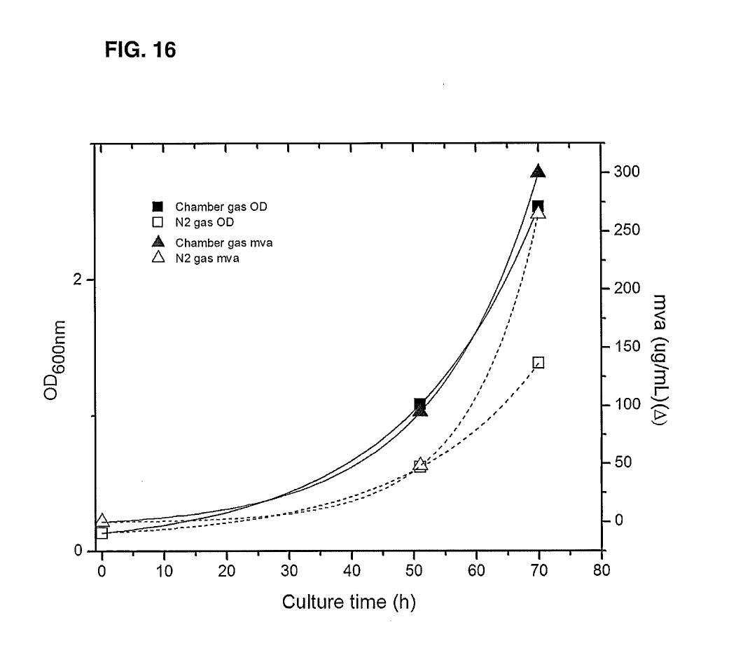

The importance of the composition of the headspace gas in determining the growth rate and mevalonate production was evaluated by placing 10 mL of pJF100 cell suspension in MES-F medium in 160 mL bottles filled with either N₂ gas or chamber gas (2% H₂, 5% CO₂ and 93% N₂). The results are shown in FIG. 16. The latter gas composition produced an approximate 2-fold more rapid growth rate compared to a headspace composed of N₂ alone. After 51 h of growth the cell density and the mevalonate concentration (analyzed by HPLC as described above) were two-fold higher in the case of the chamber gas atmosphere. After 70 h, the cell density was still nearly two-fold higher for the chamber gas atmosphere, but the mevalonate concentrations were nearly equivalent, indicating that even without the benefit of mixotrophic metabolism, the pJF 100 is capable of enhanced production of mevalonate from fructose alone when cultivated at high ratios of headspace to liquid culture.

### Liquid Chromatography/Mass Spectrometry (LC/MS) Analysis:

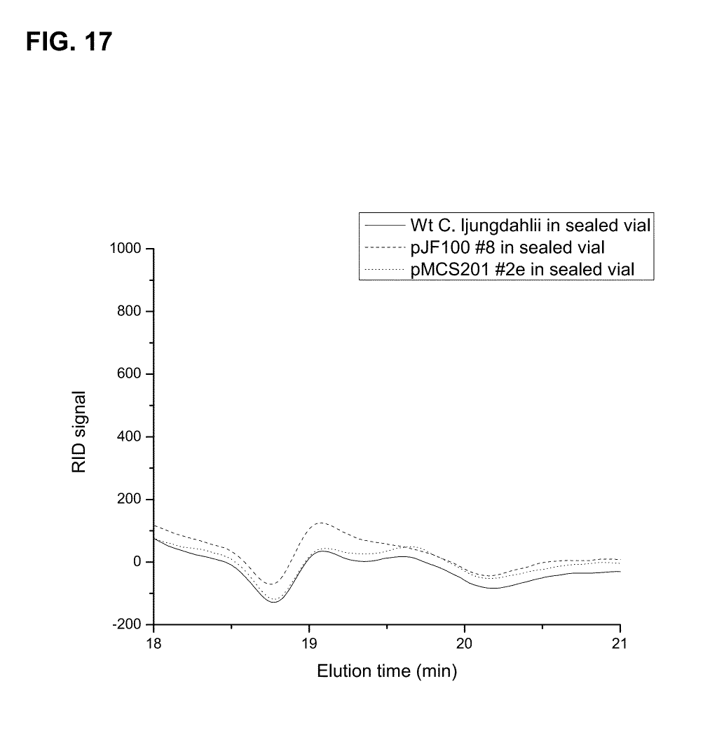

A comparison of the sealed vial conditions for the three strains (FIG. 17) showed that there was a slightly greater amplitude of the RID signal in the region where mevalonolactone elutes for the pJF100 C. ljungdahlii transformant, indicating that there might be a small amount of mevalonate produced by these cells even under sealed vial conditions. The resolution of the HPLC is insufficient to quantify the amount of mevalonate being produced under these conditions. Consequently, LC/MS, which is far more sensitive than HPLC, was used to determine mevalonate concentrations in the culture under sealed vial conditions.

pMCS201 and pJF100 transformed C. ljungdahlii strains #2, #6 and #8 were grown in MES-F medium. Plasmid was isolated from these strains and back transformed into TV3007. After growing up the TV3007 transformants in TM3 medium, the culture broth was analyzed for mevalonolactone as described above. The pJF100-containing back transformants all produced mevalonate at concentrations of approximately 1 mg/mL. There was no detectable mevalonate in the pMCS201 back transformant.

To analyze mevalonic acid by LC/MS, frozen cultures of Clostridium ljungdahlii that had been grown in sealed vials as described above were thawed at +4° C. and aliquoted into 0.5 mL portions. The samples were centrifuged, the supernatant was put aside, and the pellet was mixed with 100 μL of methanol and then centrifuged again to precipitate cell debris. Supernatants from both centrifugations were combined and buffered with 10 μL of 1M ammonium acetate (pH=7.0). The resulting sample volume was assumed to be 600 μL. Mass spectrometric analysis of the samples was performed using a TSQ Quantim triple quadrupole instrument (Thermo Scientific). System control, data acquisition, and data analysis were done with XCalibur and LCQuan software (Thermo Scientific). 10 μL samples were applied to a C18 Synergi MAX-RP HPLC column (150×2 mm, 5 μM, 80 A, Phenomenex) equipped with the manufacturer-recommended guard cartridge. The column was eluted with a gradient of 15 mM acetic acid+10 mM tributylamine in MilliQ-grade water (solvent A) and LC/MS-grade methanol (solvent B).

The 14 min gradient was as follows: t=0 min, 1% B; t=1 min, 2% B; t=8 min, 25% B; t=9 min, 70% B; t=11 min, 70% B; t=12 min, 1% B; 1=13 min, 1% B; flow rate 0.4 mL/min, column temperature 40° C. Mass detection was carried out using electrospray ionization in the negative mode at ESI spray voltage of 3.0-3.5 kV and ion transfer tube temperature of 350° C. Mevalonic acid was quantified based on intensities of the m/z=147 peak and the 147→59 SRM transition (collision energy 15 V, argon was used as the collision gas at 1.7 mTorr). Concentration of mevalonic acid in the samples was determined based on a calibration curve obtained by injecting a set of mevalonic acid standards dissolved in 20% methanol/50 mM ammonium acetate buffer (pH=7) at concentrations ranging from 0.5 to 50 μg/mL.

LC/MS showed (Table 1) that the pJF100 harboring strain of C. ljungdahlii produced on the order of 8.5 μg/mL to 54.7 μg/mL mevalonate, while the wild type C. ljungdahlii and pMCS201 transformant broths contained on the order of 0.1 μg/mL mevalonate. The broth from the cultures of the WT and pMCS201 strains were not expected to contain any mevalonate. As shown in Table 12, the MES-F culture broth alone showed a 0.1 μg/mL signal derived from the yeast extract included in the culture broth. Consequently, MvaE and MvaS, expressed in the pJF100 transformed C. ljungdahlii strains, catalyzed the production of mevalonate from acetyl-CoA, with the carbon derived from fructose under these growth conditions.

TABLE 12
Concentration of mevalonic acid in C. ljungdahlii samples
grown in sealed vials determined by LC/MS.

| | MVA, μg/mL | |
|---|---|---|
| Sample | m/z = 147 | m/z = 147-> 59 |
| C. ljungdahlii pJF100 #6 | 54.7 | 50.1 |
| C. ljungdahlii pJF100 #8 | 11.0 | 8.5 |
| C. ljungdahlii pMCS201 | 0.1 | 0.1 |
| C. ljungdahlii WT | 0.1 | 0.1 |
| MES-F medium, vial 1 | 0.1 | 0.1 |

### Example 11

### Expression of IspS and IDI in C. Ljungdahlii Crown on Fructose

Construction of Plasmid pJF200

A vector (pJF200) for expression of IspS (isoprene synthase) and Idi (isopentenyl diphosphate isomerase) using the Acetobacter woodii promoter, Awo1181gi, with a pCB102 Gram positive replicon, was constructed as follows: The vector pMTL83151, renamed pMCS201 (SEQ ID NO:12), carries the pCB102 Gram positive origin of replication, the catP marker, and the ColE1 E. coli origin of replication).

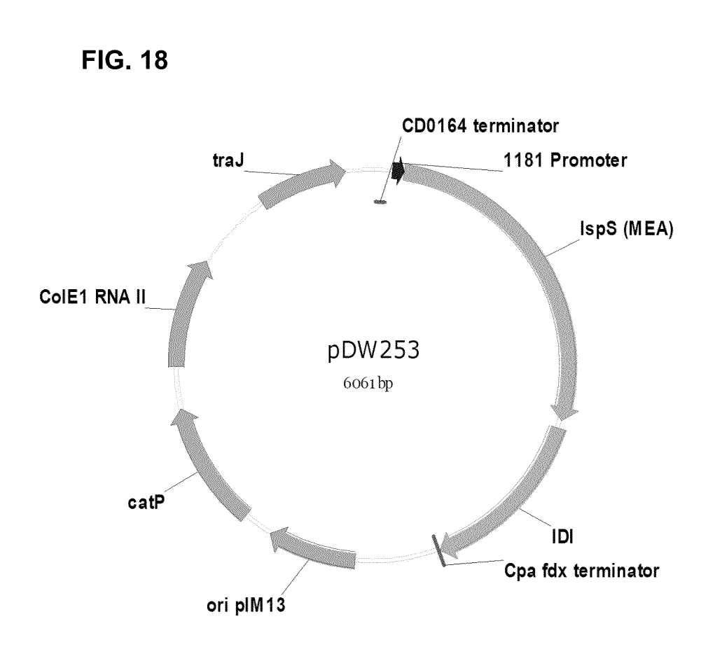

The vector pMCS337 (also called pDW253; SEQ ID NO:23; plasmid map FIG. 18) carries coding regions for ispS and idi in an operon under the control of the Awo1181gi promoter in a modular vector with a pIM13 Gram positive replicon, ColE1, E. coli replicon, and catP marker. The 1181 promoter of Acetobacter woodii present in this vector was amplified using the oligonucleotides Gi 1181 PaHgS For (SEQ ID NO:24) and Gi 1181 PaHgS Rev (SEQ ID NO:25) using the PfuUltra II DNA polymerase (Agilent Technologies) to generate a double-stranded product. The ispS coding region was derived from P. alba by truncating the 5′ end as described in U.S. Pat. No. 8,173,410, which is incorporated herein by reference, resulting in expression of the protein of SEQ ID NO:26. The truncated ispS coding region was codon optimized for expression in C. acetobutylicum (SEQ ID NO:27). The idi coding region from S. cerevisiae was codon optimized for expression in acetogens (SEQ ID NO:28; encoded amino acid sequence of SEQ ID NO:29).

The plasmid pMCS337 (pDW253) was digested with restriction enzymes PmeI and AscI (New England Biolabs, Inc, Ipswich, Mass.). The 4.4 kb vector fragment was gel purified with the QIAquick Gel Extraction Kit (Qiagen Inc, Valencia, Calif.). The digest removes the catP marker and Gram positive replicon pIM13 from the vector. The plasmid pMCS201 was digested with PmeI, AscI and ApaI (New England Biolabs, Inc, Ipswich, Mass.) and the 2.4 kb insert containing the catP marker and pCB102 replicon, was gel purified with the QIAquick Gel Extraction Kit (Qiagen Inc, Valencia, Calif.). The vector and insert were ligated with T4 DNA ligase (New England Biolabs, Inc, Ipswich, Mass.) according to manufacturer's protocol at room temperature overnight. The ligation was transformed into chemically competent E. coli Top10 cells (Invitrogen Co. Grand Island, N.Y.) following manufacturer's instructions. After outgrowth in SOC, aliquots of the transformation mix were plated onto Luria Broth (LB) plates with 15 μg/mL of chloramphenicol. Plates were incubated overnight at 30° C.

Transformants were screened by colony PCR with HotStarTaq Master Mix (Qiagen Inc, Valencia, Calif.) with primers pMCS337IspSFOR (SEQ ID NO:30), and pMCS337IspSFREV (SEQ ID NO:31) (all oligonucleotides were synthesized by Integrated DNA Technologies, Inc, Coralville, Iowa) for an approximate 1.6 kb PCR product. PCR amplification conditions were: 94° C., 15 min; 94° C. (0.5 min)-50° C. (0.5 min)-72° C. (1.5 min) for 30 cycles, and 72° C. for 10 min. Several colonies that amplified with the correct sized product were sequenced by Templiphi (GE Health Care Life Sciences, Pittsburgh, Pa.) using primers: pMCS337IspSFOR, pMCS337IspSFREV, pMCS337IDIFor (SEQ ID NO:32), PCB102F (SEQ ID NO:33), and PCB102R (SEQ ID NO:34).

After confirmation of the correct sequence, a transformant was grown at 30° C. in LB medium containing 15 μg/mL of chloramphenicol with shaking at 220 rpm. After overnight growth, the culture was centrifuged at 5,000 g for 10 minutes and the supernatant decanted. Plasmid DNA was isolated from the pelleted culture using a QIAprep Spin Miniprep Kit (Qiagen Inc, Valencia, Calif.) following manufacturer's instructions. The final elution was performed in water for use in electroporation. The resulting plasmid was named pJF200 (SEQ ID NO:35).

C. ljungdahlii was transformed by electroporation with pJF200 as previously described for pJF 100. The transformants obtained, carrying pJF200, showed no evidence, however, of IspS or Idi expression by Western blot. Plasmid isolated from these strains, used to back transform E. coli TV3007, did, however, express IspS and Idi (by Western blot) indicating that in the proper host the pJF200 plasmid was functional for expression. We conclude that while active in E. coli TV3007, the Acetobacter woodii promoter Awo1181gi was not active in C. ljungdahlii under the growth conditions used. Consequently, this promoter was replaced with the C. sporogenes Pfdx promoter that was used for MvaE and MvaS expression in Example 1.

### Construction of Plasmid pJF 100 Fdii

The ispS (Populus alba, truncated and codon optimized) and idi (S. cerevisiae, codon optimized) coding regions (both described in Example 3 above) were placed under the control of the Pfdx promoter by replacing the mvaE and mvaS coding regions in vector pJF100 with these coding regions. Primers for PCR amplification were designed using the NEBuilder for Gibson Assembly primer design tool (New England Biolabs, Inc., Ipswich, Mass.). All oligonucleotides were synthesized by Integrated DNA Technologies (Coralville, Iowa). An ˜3.5-kb DNA vector fragment which contains the Pfdx promoter was PCR amplified using pJF100 as template and primers vec100_fwd (SEQ ID NO:36) and vec100_rev (SEQ ID NO:37) using Q5® High-Fidelity 2× Master Mix (New England Biolabs, Inc., Ipswich, Mass.) under the following thermocycler conditions: initial denaturation for 30 sec at 98° C., followed by 30 cycles at 98° C. for 10 sec, 66° C. for 20 sec, and 72° C. for 1 min 45 sec, followed by final extension at 72° C. for 2 min. The resulting PCR reaction was digested with Dpn I (New England Biolabs, Inc., Ipswich, Mass.) to remove methylated template DNA for 30 min at 37° C. and heat inactivated at 80° C. for 20 min. The ˜3.5-kb PCR product was gel purified using a Zymoclean™ Gel DNA Recovery Kit (Zymo Research Corporation, Irvine, Calif.) per manufacturer's instructions.

An ˜2.7-kb DNA fragment containing coding regions for IspS and IDI was PCR amplified using pJF200 as template and primers II_insert200_fwd (SEQ ID NO:38) and II_insert200_rev (Seq ID No:39) using Q5® High-Fidelity 2× Master Mix (New England Biolabs, Inc., Ipswich, Mass.) under the following thermocycler conditions: initial denaturation for 30 sec at 98° C., followed by 30 cycles of 98° C. for 10 sec, 70° C. for 20 sec, and 72° C. for 1 min 20 sec, followed by final extension at 72° C. for 2 min. The resulting PCR reaction was digested with Dpn I (New England Biolabs, Inc., Ipswich, Mass.) to remove methylated template DNA for 30 min at 37° C. and heat inactivated at 80° C. for 20 min. The ˜2.7-kb PCR product was gel purified using a Zymoclean™ Gel DNA Recovery Kit (Zymo Research Corporation, Irvine, Calif.) per manufacturer's instructions.

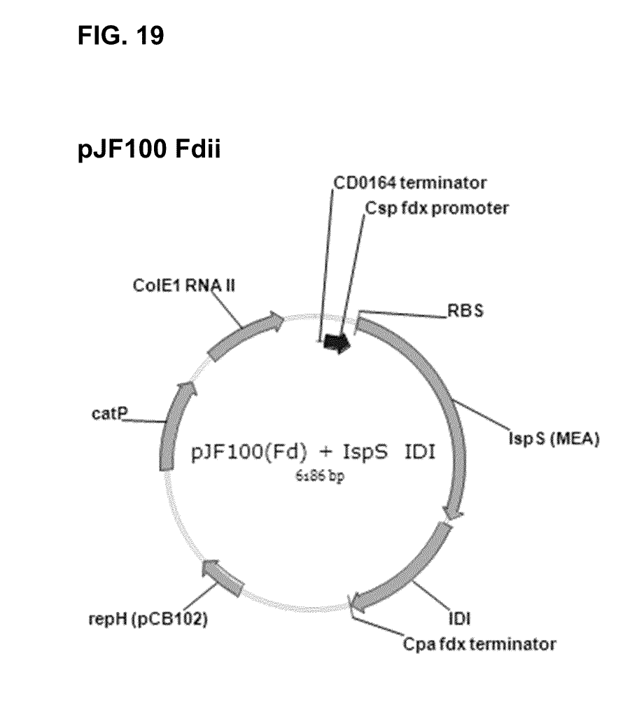

The ˜2.7-kb ispS/idi insert fragment and the ˜3.5-kb vector fragment were combined, using 100 ng vector DNA and at least 2-fold excess insert, and assembled using a Gibson Assembly® Cloning Kit (New England Biolabs, Inc., Ipswich, Mass.), following manufacturer's instructions. After incubation for 15 min at 50° C., a chilled aliquot of the resulting reaction was used to transform High Efficiency NEB 5-alpha Competent E. coli (New England Biolabs, Inc., Ipswich, Mass.) following manufacturer's instructions. After 2 hour outgrowth in SOC at 30° C., aliquots of the transformation mix were spread onto Luria Broth (LB) plates containing 5 μg/mL of chloramphenicol. Plates were incubated overnight at 30° C. Transformants were screened through colony PCR screening using HotStarTaq Master Mix Kit (Qiagen, Inc., Valencia, Calif.) with primers Fdx For1 (SEQ ID NO:40) and Isp seq R2 (SEQ ID NO:47), following manufacturer's instructions. Plasmid DNA was prepared from cell pellets using a QIAprep Spin Miniprep Kit (Qiagen, Inc., Valencia, Calif.) and sequenced using primers UP mcs Mint (SEQ ID NO:41), Isp seq F1 (SEQ ID NO:42), Isp seq F2 (SEQ ID NO:43), Isp seq F3 (SEQ ID NO:44), Isp seq F4 (SEQ ID NO:45), Isp seq R1 (SEQ ID NO:46), Isp seq R2 (SEQ ID NO:47), Isp seq R3 (SEQ ID NO:48), Id seq F1 (SEQ ID NO:49), Id seq F2 (SEQ ID NO:50), Id seq F3 (SEQ ID NO:51), Id seq R1 (SEQ ID NO:52), Id seq R2 (SEQ ID NO:53), and repH seq F1 (SEQ ID NO:54). The resulting ˜6.2-kb plasmid was named pJF100 Fdii (FIG. 19; SEQ ID NO:55).

Electrocompetent C. ljungdahlii cells were transformed with two different preparations of pJF 100 Fdii plasmid, called plasmid 1 and plasmid 3, isolated from Top 10 cells as described earlier. Four colonies, two from each of the two plasmid preps were picked from the enriched MES-F+5 μg/mL thiamphenicol plates and were restreaked on MES-F+5 μg/mL thiamphenicol plates. These were used to inoculate 50 mL cultures of MES-F+5 μg/mL thiamphenicol in 160 mL bottles. Cultures of pJF100 Fdii #1, plasmid 1, #4 plasmid 1, #9 plasmid 3 and #11 plasmid 3 were grown to an OD₆₀₀ of 0.724, 0.768, 0.610 and 0.670. The cell suspensions were poured into 50 mL conical centrifuge tubes, which were sealed, removed from the anaerobic chamber and spun at 5000 rpm for 10 min at 5° C. in a HS-4 Sorvall rotor. The tubes were returned to the anaerobic chamber, the supernatants poured off and the pellets resuspended in 27 mL each of PBS. The tubes were again removed from the anaerobic chamber and spun as before in the HS-4 rotor. The tubes were returned to the anaerobic chamber and the supernatants poured off. The tubes were removed from the anaerobic chamber, frozen in liquid N₂ and stored in a −80° C. freezer.

In preparation for Western blotting, the pellets were thawed and resuspended in 1.4, 1.5, 1.2 and 1.3 mL, respectively, of PBS containing 100 μg/mL PMSF. The different volumes were used to assure approximately equal cell densities. The cell suspensions were passaged three times through a French pressure cell at 20,000 psi at ˜5° C. A portion of the lysates were used immediately for the Western blotting and the remainder aliquotted oμL quick frozen in liquid N₂ and stored in a −80° C. freezer. Fifteen μL of lysate was mixed with 5 μL of LDS sample buffer (Invitrogen, Carlsbad Calif., Cat. No. NP0008) for each lane of each of three gels: two Western blots and one stained with Simple Blue SafeStain.

Transformants of E. coli TV3007 were prepared as described earlier with plasmid isolated from two C. ljungdahlii pJF200 Fdii transformants named #1LD and 2LD. IspS and Idi were expressed from the Awo1181 promoter in E. coli TV3007 and were used as markers for IspS and Idi. These transformants were grown up as described earlier and harvested and solubilized with LDS sample buffer as before.

The electrophoresis gel system was run as described in Example 1 and the gels were transferred, incubated with antibody and then with anti-rabbit antibody also as in Example 1. The primary antibodies used for each of the two gels were Rabbit polyclonal antibodies made by ProSci™ (Poway, Calif.): anti IspS (P. alba) and Rabbit anti idi (S. cerevisiae) and used at a dilution of 1:1000 in blocking solution. The secondary antibody solution was as in Example 1.

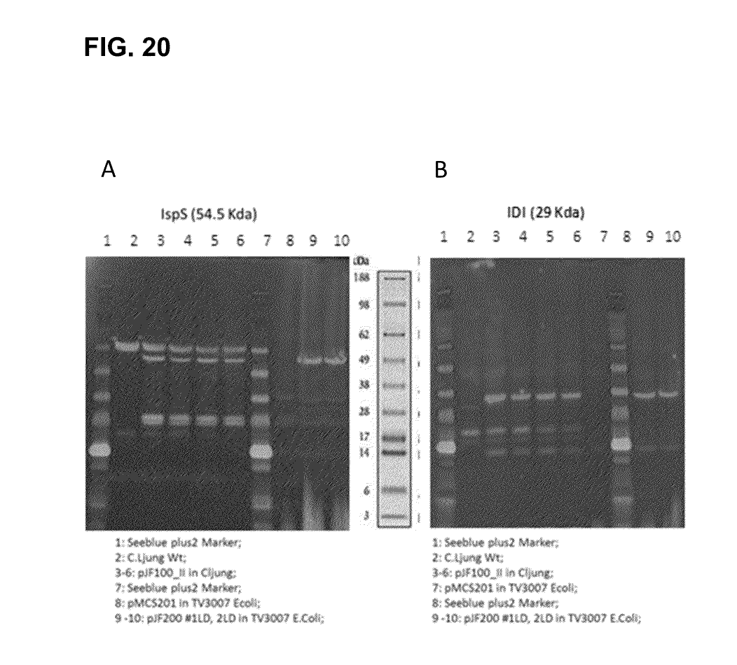

The Western blots shown in FIG. 20 detected IspS (20A) and Idi (20B) polypeptides in the E. coli TV3007 pJF200 transformants and in the C. ljungdahlii pJF100 Fdii transformants, and indicated good levels of expression of both proteins. There was, however, considerable proteolysis of IspS in the C. ljungdahlii transformants generating two fragments of ˜27-8 kDa. In an effort to determine the site of proteolytic cleavage, the pJF 100 Fdii plasmid was modified such that expression would result in a hexahistidine tag at either the N- or C-terminal end of IspS. That way the two proteolytic fragments could be isolated by affinity chromatography and sequenced to determine the cleavage site.

### HexaHistidine Tagged Isps

Plasmid pJF 100 Fdii was used as template for the addition of N and C terminal 6×His tag coding sequences to the isoprene synthase (ispS) coding region. His tags were inserted using QuickChange II XL Site Directed Mutagenesis Kit (Catalog #200521 Agilent Technologies, Santa Clara, Calif.). Each PCR reaction contained 25 ng DNA template and 125 ng each of the forward and reverse primer in 50 μl total volume according to the manufacturer's instructions. Primers to insert the N-terminus His tag were N-Forward and N-Reverse (SEQ ID NOs:56 and 57, respectively). Primers to insert the C-terminus His-tag were C-Forward and C-Reverse (SEQ ID NOs:58 and 59, respectively). All oligonucleotide primers were synthesized by Integrated DNA Technologies (Coralville, Iowa). PCR was performed for 18 cycles as follows: step 1 95° C. 1 minute; step 2 94° C. 50 sec; repeat steps 2-4 18 times; step 3 60° C. 50 sec; step 4 68° C. 7 min; step 5 68° C. 7 min; step 6 4° C. hold.

At the completion of PCR cycling, 1 μl DpnI restriction enzyme was added to the amplification reaction to remove methylated template DNA. Digestion was at 37° C. for 1 hour. Following digestion at 37° C. each reaction was placed on ice. From each reaction tube 3 μl was transferred to a vial of One Shot Top 10 chemically competent E. coli (Invitrogen cat#4040-03, Grand Island, N.Y.) on ice. The transformations were incubated on ice for 30 minutes. Cells were heat pulsed at 42° C. for 30 seconds and placed on ice for 2 minutes. 250 μl of SOC medium (Invitrogen) was added to the transformation mix and the mix was incubated at 30° C. for 2 hours. For each transformation 150 μl was spread on LB chloramphenicol 5 μg/ml plates and the plates incubated overnight at 30° C. Colonies containing putative N and C-terminal 6× his tags were used to inoculate 2 ml LB chloramphenicol 5 μg/mL each for overnight growth at 30° C. Plasmid DNA was prepared using a Qiaprep Spin Miniprep it (Catalog #27016, Qiagen Valencia, Calif.). The addition of the N and C-terminal 6× his tags was verified by sequencing using primers FdxF1 and IDIR2 (SEQ ID NOs:60 and 61, respectively).

Electrocompetent C. ljungdahlii cells were transformed with preparations of the pJF 100 Fdii N-terminally His tagged and C-terminally His tagged plasmids, (pJF 100 Fdii His N and pJF100 Fdii His C, respectively) isolated from Top 10 cells as described earlier. Four colonies, two from each of the two plasmid preps were picked from the enriched MES-F+5 μg/mL thiamphenicol plates and were restreaked on MES-F+5 μg/mL thiamphenicol plates. These were used to inoculate 60 mL cultures of MES-F+5 μg/mL thiamphenicol in 160 mL bottles. Cultures of pJF100 Fdii N-term His tagged #1 and #2 and C-term His tagged #1 and #4 were grown on MES-F medium at 37° C. to an OD₆₀₀ of 0.806, 0.678 and 0.932 and 0.722, respectively. Forty-five mL of each of the cell suspensions were poured into 50 mL conical centrifuge tubes, which were sealed, removed from the anaerobic chamber and spun at 5000 rpm for 10 min at 5° C. in a HS-4 Sorvall rotor. The tubes were returned to the anaerobic chamber, the supernatants poured off and the pellets resuspended in 25 mL each of PBS. The tubes were again removed from the anaerobic chamber and spun as before in the HS-4 rotor. The tubes were returned to the anaerobic chamber and the supernatants poured off. The tubes containing the pelleted cells were removed from the anaerobic chamber, frozen in liquid N₂ and stored in a −80° C. freezer.

In preparation for Western blotting, the pellets were thawed and resuspended in 1.5, 1.27, 1.75 and 1.35 mL, respectively, of PBS containing 100 μg/mL PMSF. The different volumes were used to assure approximately equal cell densities. The cell suspensions were passaged three times through a French pressure cell at 20,000 psi at ˜5° C. A portion of the lysates were used immediately for the Western blotting and the remainder aliquotted oμL quick frozen in liquid N₂ and stored in a −80° C. freezer. Fifteen μL of lysate was mixed with 5 μL of LDS sample buffer (Invitrogen, Carlsbad Calif., Cat. No. NP0008) for each lane of each of three gels: two Western blots and one stained with Simple Blue SafeStain.

Transformants of E. coli TV3007 were prepared as described earlier with plasmid isolated from C. ljungdahlii pJF200 transformants #1LD and 2LD. These were grown up as described and harvested and solubilized with LDS sample buffer as before to be used as markers for IspS and Idi.

The electrophoresis gel system was run as described earlier and the gels were transferred, incubated with antibody and then with anti-rabbit antibody as before.

The primary antibodies were anti IspS and anti Idi, described above, and were used at a dilution of 1:1000 in blocking solution. The secondary antibody solution was as before.

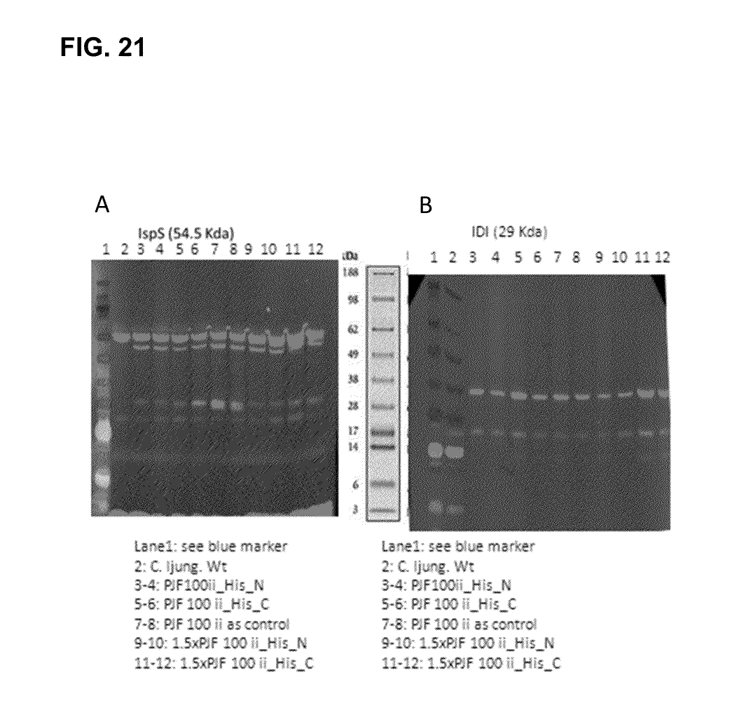

The Western blot shown in FIG. 21A detected N- and C-terminally His tagged IspS in the C. ljungdahlii transformants, and indicated good levels of expression of IspS independent of the addition of the terminal His tags. The proteolysis of IspS generating fragments of ˜27-8 kDa was much reduced in the His tagged variants as compared to IspS without the His tags. The intensity of the parent band appeared to be about the same with and without the His tags. The Western blot shown in FIG. 21B detected the Idi polypeptide in the C. ljungdahlii pJF100 Fdii transformants with his-tagged IspS, and indicated good levels of expression of the protein.

### Example 12

### Production of Isoprene in C. Ljungdahlii Crown on Fructose

FIG. 3 shows various pathways in obligate anaerobes expressing a heterologous isoprene synthase and a heterologous isopentenyl diphosphate isomerase.

### Fructose to Isoprene

MES-F medium was inoculated from a frozen stock of WT C. ljungdahlii, two days before assaying for isoprene, and incubated in an anaerobic chamber on an incubator shaker at 37° C. and 110 rpm. MES-F+5 μg/mL thiamphenicol medium was inoculated from petri plate cultures of pJF100 Fdii#1 plasmid 1 and #9, plasmid 3 three days before analyzing for isoprene. The day before assaying for isoprene, the wild type culture was diluted 1:10 with MES-F and 10 ml (at 0.082 OD₆₀₀) were placed in 20 mL vials (Agilent 20 mL headspace vials with screw caps with silicone/PTFE septa, VWR Part Nos. 5188-2753 and 5188-2759, respectively). Ten mL of each of the two pJF100 Fdii transformant cultures were also transferred to 20 mL Agilent vials, the day before assaying for isoprene. All three cultures were then incubated overnight in the anaerobic chamber on an incubator shaker at 37° C. and 110 rpm. The OD of the cultures at the time of headspace measurement was 1.788, 0.392 and 2.096 for the WT, pJF100 Fdii #1 plasmid 1 and pJF100 Fdii #9 plasmid 3, respectively. The one-day old headspace of each of the vials was sampled with a 1 mL gas-tight syringe (Hamilton) and 1 mL was injected in splitless mode into the injection port of a 7890A Agilent GC coupled to an Agilent 7975C mass spectrometer for each sample. The signals and spectra obtained were compared to a 0.3 mL injection of isoprene standard (1090 ppm isoprene in N₂).

The isoprene standard showed a retention time of 1.73 minutes in the GC elution profile, with mass spectrum m/z features of 68, 67, 54 and 39. The C. ljungdahlii WT and pJF 100 Fdii transformants both showed a peak with retention time matching that of the isoprene standard. In addition, the mass spectra of the WT and transformant peaks with elution time from 1.712 to 1.768 match the isoprene spectrum, clearly indicating the presence of isoprene. Single ion monitoring at m/z=67 for the WT and the two pJF100 Fdii transformants showed that the amount of isoprene in the transformants was 4 to 5-fold greater than in the WT.

### Example 13

### Growth of C. Ljungdahlii on Syngas

### Adaptation to Syngas-Dependent Growth

WT C. ljungdahlii, and the pJF100 and pJF100 Fdii C. ljungdahlii transformants were all grown on MES-F medium in the anaerobic chamber after which the cell suspensions were diluted to 0.05 OD₆₀₀ into 10 mL MES-0.1F medium (identical to MES-F but with 1 g/L fructose instead of 10 g/L) in 160 mL bottles under a syngas atmosphere (35% CO, 36% H₂, 18% CO₂ and 11% Ar). In this and the following Examples, the syngas atmosphere was charged at a pressure of 10 psig. Cultures of the transformants were supplemented with thiamphenicol (5 μg/mL) to assure maintenance of plasmid. The WT, pJF 100 transformant and pJF100 Fdii transformant cultures lagged for 2 d, 0 d and 3 d, respectively, before rapidly growing to an OD₆₀₀ of 0.5-0.7 OD₆₀₀ in one day, after which there was no further growth. These cultures were then diluted once again to 0.05 OD₆₀₀ in 10 mL MES-0F medium (identical to MES-F but without fructose) in 160 mL bottles under a syngas atmosphere. Thiamphenicol (5 μg/mL) was added to the pJF100 and the pJF100 Fdii transformant cultures. The WT culture grew very slowly, attaining an OD₆₀₀ of 0.240 after one week. The pJF 100 culture also grew very slowly attaining an OD600 of 0.154 after 5 d. The pJF 100 Fdii culture reached an OD₆₀₀ of 0.069 after 3 d. At these time points the cells in the cultures were spun down at 5000 rpm for 10 min in an HS-4 rotor (Sorvall) and plated onto petri plates containing MES-0F (+5 μg/mL thiamphenicol for the transformants) in the anaerobic chamber. The plates were then stored in an Oxoid jar under the syngas atmosphere at room temperature for 3 weeks. Four colonies each appeared for the WT and pJF 100-transformed strains. There was no growth on the pJF100 Fdii transformant plate. The WT and pJF100 colonies were picked and restreaked onto MES-0F plates (+5 μg/mL thiamphenicol for pJF100) and stored in an Oxoid jar under the syngas atmosphere at room temperature at which point they continued to grow.

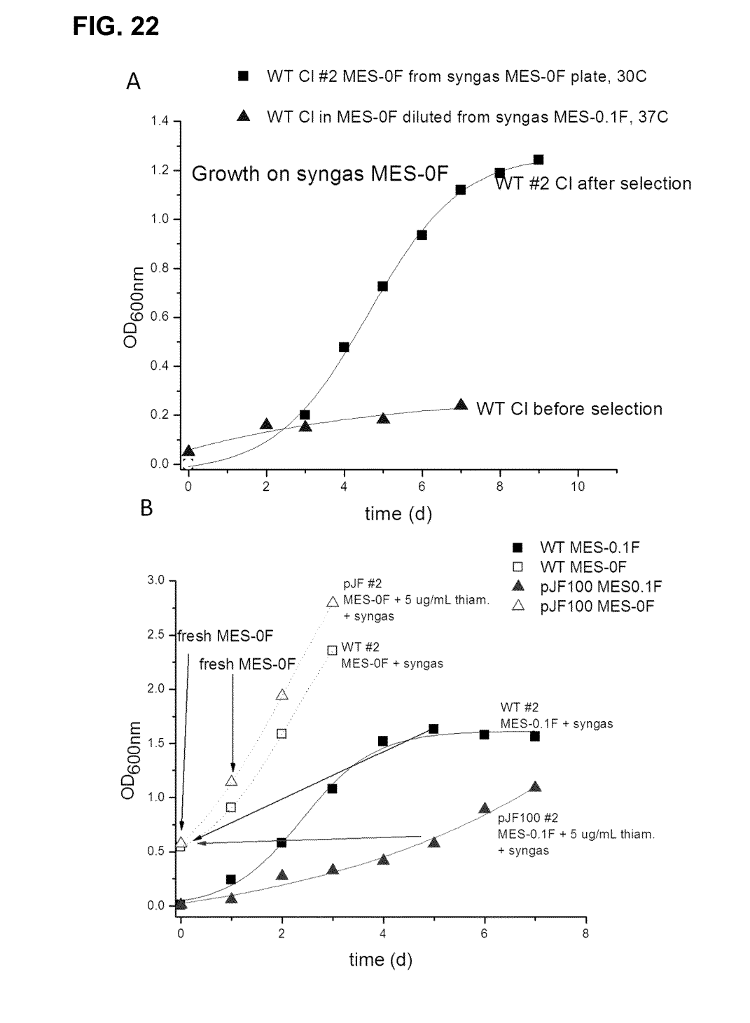

FIG. 22A shows a comparison of growth of WT cells on MES-0F under a syngas atmosphere before and after adaptation to syngas. The pre-selection WT was diluted to 0.05 OD₆₀₀ in 10 mL MES-0F+syngas (37° C.) in a 160 mL sealed bottle following 37° C. growth on MES-0.1F to 0.766 OD₆₀₀. The syngas-adapted WT was inoculated from a room temperature MES-0F petri plate into 10 mL MES-0F medium in a 160 mL bottle under a syngas atmosphere at 30° C. The syngas atmosphere was refreshed at each cell density measurement.

While before selection the WT cells were incapable of sustained growth on MES-0F medium, once adapted, they were capable of doubling in less than 24 h. Similar behavior was observed for the pJF 100#2 as show in FIG. 22B, where the doubling time was less than 24 h for both the WT and the pJF 100 adapted strains. Here both the WT and the pJF 100 syngas-adapted strains were inoculated from MES-0F+syngas plates into liquid MES-0.1F medium under a syngas atmosphere. Thiamphenicol (5 μg/mL) was present in both the solid and liquid media for the pJF100 strain. The WT MES-0.1F culture at OD₆₀₀=1.630 and the pJF100 MES-0.1F culture at OD₆₀₀=0.574 were spun down at 5000 rpm for 10 min in a Sorvall HS-4 rotor in sealed conical centrifuge tubes and resuspended at OD₆₀₀ 0.543 and 0.574, respectively, in 5 mL MES-0F (+5 μg/mL thiamphenicol) for pJF 100 and placed in 160 mL sealed bottles under a syngas atmosphere. The next day, the cells were again spun down and resuspended in the same volume of MES-0F medium at the same cell density that they were at on that day. After that only the syngas atmosphere was refreshed each day and the OD₆₀₀ was followed. The WT and the pJF100 transformant show approximately equal rates of growth on MES-0F, with 5 μg/mL thiamphenicol present in the case of the transformant.

### Example 14

### Production of Mevalonate in C. Ljungdahlii Crown on Syngas

### Conversion of Syngas to Mevalonate

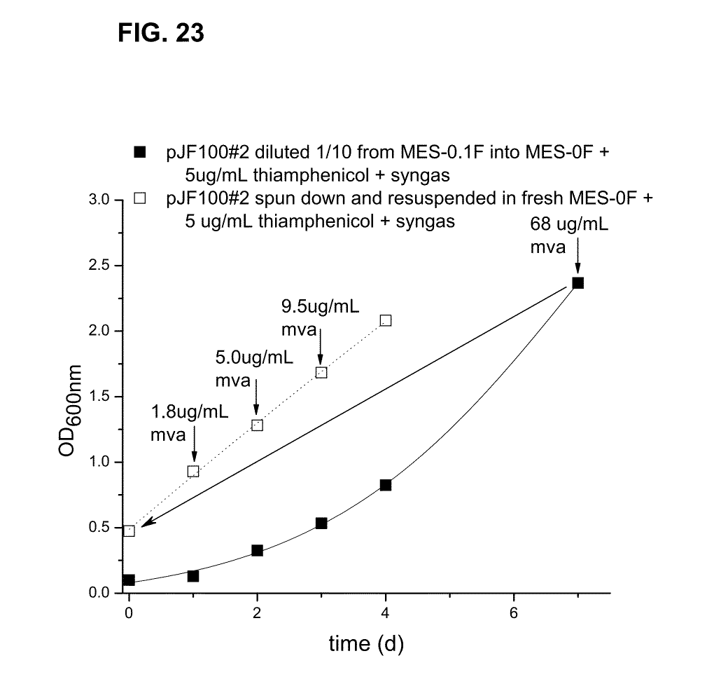

Strain pJF100#2 was cultivated initially in MES-0.1 F medium+5 μg/mL thiamphenicol under a syngas atmosphere to 1.090 OD₆₀₀ and then diluted to 0.01 OD₆₀₀ in MES-0F medium+5 μg/mL thiamphenicol under a syngas atmosphere. After growing up to an OD₆₀₀ of 2.368, the cells were spun down and resuspended anaerobically in fresh MES-0F medium+5 μg/mL thiamphenicol. Samples were taken on Day 7 of the first passage on MES-0F under syngas and on Days 1, 2 and 3 of the second passage on MES-0F under syngas. The mevalonate concentrations were determined by LC/MS as described in Example 2. FIG. 23 shows growth followed by OD₆₀₀ as well as mevalonate concentrations for the first passage end point and the second passage cultures. The mevalonic acid concentrations in FIG. 23 are the average of the m/z=147 peak and the 147→59 SRM transition, which are given separately in Table 13.

TABLE 13
LC/MS determination of mevalonate concentration in
pJF100 C. ljungdahlii transformants grown on syngas

| | | Conc. μg mva/mL | |
|---|---|---|---|
| Sample | OD₆₀₀ | 147 → 59 | 147 |
| WT | 1.07 | 0.39 | 0.32 |
| Day 7, 1^{st} passage on MES-0F | 2.368 | 75.82 | 60.92 |
| Day 1, 2^{nd} passage on MES-0F | 0.938 | 2.01 | 1.62 |
| Day 2, 2^{nd} passage on MES-0F | 1.28 | 5.34 | 4.62 |
| Day 3, 2^{nd} passage on MES-0F | 1.684 | 10.22 | 8.83 |

Metabolic (growth and mevalonate production) dependence on syngas in the absence of fructose for syngas adapted pJF100 C. ljungdahlii transformants

To demonstrate that the C. ljungdahlii pJF100 transformant is converting syngas to cell biomass and product, the cells were cultivated on MES-0F medium at 30° C. under either an atmosphere of syngas (35% CO, 36% H₂, 18% CO₂ and 11% Ar) or an atmosphere of N₂ alone.

Four mL of two different MES-0F cultures of pJF 100 transformed C. ljungdahlii cells were placed in Supelco vials (Supelco Part. No 27159, Bellefonte, Pa.) with a 12.5 mL headspace of either syngas or N₂ gas and incubated for 26 h at 30° C. on an incubater-shaker. Three mL of the same two different MES-0F cultures of pJF100 transformed C. ljungdahlii cells were also placed in sealed 160 mL bottles under an atmosphere of syngas or N₂ gas. The extent of growth after 26 h in the Supelco vials and after 28 h in the 160 mL bottles are shown in Table 14. There was significantly more growth in the presence of the syngas atmosphere than there was under a N₂ atmosphere, indicative of a dependence on syngas for growth.

TABLE 14
Overnight growth at 30° C. of two cultures of C. ljungdahlii
transformed with pJF100 in 16.5 mL Supelco vials or
160 mL bottles under an atmosphere of syngas (35% CO, 36% H₂,
18% CO₂ and 11% Ar) or N₂ gas, both at 10 psi.

| | OD₆₀₀ at | OD₆₀₀ at 26 h (Supelco vial) |
|---|---|---|
| Sample | 0 time | and 28 h (160 mL bottle) |
| Syngas culture 1—Supelco vial | 0.500 | 0.838 |
| Syngas culture 4—Supelco vial | 0.500 | 0.806 |
| N₂ gas culture 1—Supelco vial | 0.500 | 0.520 |
| N₂ gas culture 4—Supelco vial | 0.500 | 0.520 |
| Syngas culture 1—Bottle | 0.324 | 0.608 |
| Syngas culture 4—Bottle | 0.152 | 0.374 |
| N₂ gas culture 1—Bottle | 0.324 | 0.342 |
| N₂ gas culture 4—Bottle | 0.152 | 0.213 |

The cultures grown in the Supelco vials for 26 h (described above) were analyzed for the percent consumption of CO (as described below) in that period of time and the mole ratio of CO₂ produced/CO consumed, which are given in Table 15.

TABLE 15
Percent consumption of CO and the mole ratio of CO₂
produced/CO consumed for the two 30° C. overnight cultures
in Supelco vials of C. ljungdahlii transformed with pJF100.

| | | Mole ratio |
|---|---|---|
| | % of CO | CO₂ produced |
| Sample | consumed | C̅O̅ c̅o̅n̅s̅u̅m̅e̅d̅ |
| Syngas culture 1—Supelco vial | 52.65 ± 1.64 | 0.237 ± 0.090 |
| | | (3 measurements) |
| Syngas culture 4—Supelco vial | 51.90 ± 0.14 | 0.185 ± 0.010 |
| | | (2 measurements) |

That the increase in biomass in the overnight cultures was dependent on having syngas in the headspace and that the mole ratio of CO₂ produced/CO consumed was less than one, both indicate that the syngas-adapted C. ljungdahlii pJF100 transformants were metabolizing syngas (fixing CO and CO₂) and that CO and CO₂ were the source of the biomass and of the mevalonate generated.

Acetate was assayed in media of WT and a C. ljungdahlii pJF100 transformant grown on MES-0.1F+syngas and on MES-0F+syngas, similarly to as described above. Both strains showed reduced levels of acetate when grown in MES-0F+syngas as compared to acetate levels when grown on MES-0.1F+syngas. For WT the acetate level was about 5-fold reduced and for the C. ljungdahlii pJF 100 transformant the acetate level was about 2-fold reduced. Thus growth in syngas without fructose resulted in a substantial decrease in net production of acetate.

### Example 15

### Production of Isoprene in C. Ljungdahlii Crown on Syngas

Adaptation of pJF100 Fdii N- and C-Terminally his Tagged IspS Transformants

Both of the pJF 100 Fdii N- and C-terminally His tagged IspS transformants were grown on 10 mL MES-F+5 μg/mL thiamphenicol in the anaerobic chamber at 37° C. and then diluted to 0.01 OD₆₀₀ in 10 ml MES-0.1F medium+5 μg/mL thiamphenicol in 160 mL sealed bottles containing syngas. These were then grown up in an incubator shaker at 30° C. to 0.5 OD and then spun down and resuspended in MES-0F+5 μg/mL thiamphenicol in sealed 160 mL bottles under a syngas atmosphere. These cultures were grown at 30° C. and repassaged multiple times on MES-0F+5 μg/mL thiamphenicol under a syngas atmosphere under the same conditions over a period of four weeks.

### Consumption of CO in Syngas

These cultures and a syngas-adapted WT culture grown on MES-0F (from Example 13) were grown to OD₆₀₀ of 1.132, 0.856, and 0.960 for the WT, pJF100 Fdii N-terminal His tagged IspS transformant, and pJF100 Fdii C-terminal His tagged IspS transformant, respectively. These cultures were diluted to 0.5 OD₆₀₀ using MES-0F+5 μg/mL thiamphenicol, 4 mL of which were placed in Supelco 16.5 mL vials (Supelco Part. No 27159, Bellefonte, Pa.) under a syngas atmosphere sealed with a septum-containing cap and placed on a shaker incubator at 30° C. overnight. After 20 h, the headspace was sampled using a GC to analyze the composition of the syngas. The needle (Part. No. 779-03, Hamilton Co. Reno, Nev.)) of a 100 μL gas-tight Hamilton syringe (Model 1710) was inserted through the septum and 100 μL was withdrawn and injected into a splitless injection port of an Agilent Model 7890B Gas Chromatograph at 150° C. using ultra high purity He as the carrier gas. A Varian CP7429 Select Permanent Gas/CO₂ column was run isothermally at 45° C. to separate the components of the sample (CO, H₂, CO₂ and Ar), which were detected using a thermal conductivity detector maintained at 200° C.

Results given in Table 16 indicate that the consumption of CO in the syngas increased with the production of biomass in this experiment over a 20-h period.

TABLE 16
Growth and consumption of CO in adapted C. ljungdahlii

| | | | moles CO₂ produced |
|---|---|---|---|
| | | | moles CO consumed |
| Syngas-adapted | % of CO | | Average of two |
| strains strains | consumed | OD₆₀₀ change | measurements |
| N-term His-tagged | 42 | 0.500 to 0.888 | 0.44 ± 0.07 |
| Isps | | | |
| C-term His-tagged | 52 | 0.500 to 0.952 | 0.34 ± 0.35 |
| Isps | | | |
| WT | 58 | 0.500 to 1.034 | 0.41 ± 0.12 |

A comparison with the starting composition of the syngas in two separate experiments with these strains, given in Table 16, indicated that more moles of CO were consumed than were liberated in the form of CO₂. The increased consumption of CO with greater OD₆₀₀ increase and a mole ratio of less than one for CO₂ produced per mole of CO consumed both indicate that CO is being fixed and incorporated into the biomass and synthesis products of the cells.

### Solid Phase Microextraction (SPME) of the Vial Headspace to Confirm the Production of Isoprene

A 75 μm Carboxen/PDMS Fused Silica SPME fiber was used in this study. The SPME fiber was conditioned by heating at 275° C. under helium flow for 15 minutes. After each conditioning period, the SPME fiber was analyzed by GC/MS to verify that the fiber did not contain residual isoprene. The conditioned SPME fiber was then introduced through the septum into the Supelco vial containing the 4 mL of C. ljungdahlii culture. The fiber was positioned in the headspace, ˜1 cm above the broth, and allowed to equilibrate for 15 minutes. After the equilibration period, the fiber was removed from the vial and immediately inserted into the inlet of an Agilent 7890A/5975C GC/MS instrument. The inlet temperature was 250° C. The fiber was held in the inlet for 2 minutes. Desorbed material from the fiber was collected onto the chromatographic column (RTX-1 60 m×0.320 mm×3 μm) by cooling the oven to −35° C. After the desorption period, the fiber was removed from the inlet and the oven was heated at 20° C. per minute to a final temperature of 250° C. The inlet was operated in both split (20:1) and splitless modes.

The ion chromatogram for the WT, the pJF100 Fdii N-term His tagged IspS transformant, and the pJF 100 Fdii C-term His tagged IspS transformant each showed an elution peak centered at 10.25 min. The mass spectra for each of these peaks was compared to a reference isoprene mass spectrum and all showed a good quality fit with the reference isoprene spectrum, with the quality of the fit increasing with the increase in concentration of the eluted isoprene.

A comparison of the extracted ion chromatograms at m/z=67 showed that the amplitude of the N-term His-tagged IspS transformant signal was substantially larger than that of the WT and the pJF100 Fdii C-term His tagged IspS transformant. The latter two were fairly similar. Integrated peak intensities for the isoprene signals detected in the extracted ion chromatograms at m/z=67 and at a retention time of 10.25 min for the WT, pJF 100 Fdii C-term His-tagged IspS transformant and the pJF100 Fdii N-term His-tagged IspS transformant are given in Table 17. The average of two runs, the first using split mode (20:1) and the second using splitless mode showed that the pJF100 Fdii N-term His-tagged IspS strain produced approximately 5 times more isoprene than the WT. The Fdii C-term His-tagged IspS strain produced about as much isoprene as the WT, within the noise of the measurement.

TABLE 17
Integrated isoprene peak intensity for the WT, pJF100 Fdii C-term
His-tagged IspS and the pJF100 Fdii N-term His-tagged IspS
for the isoprene signals detected in the extracted ion chromatograms
at m/z = 67 and at a retention time of 10.25 min.

| | Isoprene Peak Intensity | | | |
|---|---|---|---|---|
| | (EIC for m/z = 67 and RT = 10.25 min) | | | Ratio |
| | Wild Type | C-TERM | N-TERM | N-TERM to |
| Injection Mode | #9 | HIS #8 | HIS #7 | Wild Type |
| Splitless— | 17430 | 11150 | 71467 | 4.1 |
| 2^{nd} extraction | | | | |
| Split (20:1)— | 2377 | 520 | 14828 | 6.2 |
| l^{st} extraction | | | | |
| Average | — | | — | 5.2 |

What is claimed is:

1. Recombinant obligate anaerobic bacterial acetogen cells comprising:
a) at least one heterologous nucleic acid molecule encoding an isoprene synthase polypeptide;
b) at least one heterologous nucleic acid molecule encoding an isopentenyl diphosphate isomerase polypeptide,
wherein the nucleic acid molecules of a) and b) are operably linked either separately or in an operon to a promoter that is functional in the cells, and
wherein the cells produce more isoprene when grown in substantially oxygen-free culture conditions comprising synthesis gas as energy and/or carbon source, as compared to the cells without the heterologous nucleic acids.

2. The cells of claim 1, wherein the synthesis gas is used as energy and/or carbon source.

3. The cells of claim 1, wherein the cells are selected from the group consisting of Clostridium ljungdahlii, Clostridium aceticum, Moorella thermoacetica, Clostridium autoethanogenum, Eurobacterium limosum, Clostridium carboxydivorans, Peptostreptococcus productus, Rhodospirillum rubrum, Desulfitobacterium hafniense, Aecetoanaerobium notera, Butyribacterium methylotrophicum, Thermoanaerobacter kivui, Eubacterium limosum, Peptostreptococcus productus, and Acetobacterium woodi.

4. The cells of claim 1, wherein the isoprene synthase polypeptide is a variant of a naturally occurring isoprene synthase polypeptide, which belongs to the group EC 4.2.3.27, wherein the variant isoprene synthase polypeptide has an improvement selected from the group consisting of improved catalytic activity, improved solubility, increased resistance to degradation, increased resistance to cleavage, and combinations thereof, as compared to the naturally occurring isoprene synthase polypeptide.

5. The cells of claim 4, wherein the variant isoprene synthase polypeptide comprises one or more modifications selected from the group consisting of substitution, addition, deletion, and truncation.

6. The cells of claim 5 wherein the variant comprises a truncation of the native N-terminus and an addition of a heterologous oligopeptide in the N-terminal end.

7. The cells of claim 4, wherein the variant is of a plant isoprene synthase polypeptide.

8. The cells of claim 7, wherein the plant is selected from Pueraria, Populus, and a Popuius alba×Populus tremula hybrid.

9. The cells of claim 1, wherein the promoter is from a cell of the genus Clostridium.

10. The cells of claim 1, wherein the isopentenyl diphosphate isomerase polypeptide belongs to the group EC 5.3.3.2.

11. The cells of claim 10, wherein the isopentenyl diphosphate isomerase polypeptide is from a yeast.

12. The cells of claim 1, further comprising one or more introduced nucleic acids encoding one or more DXP pathway polypeptide(s), wherein the DXP pathway polypeptide(s) have increased expression as compared to the cells without the introduced nucleic acids encoding DXP pathway polypeptide(s).

13. The cells of claim 12, wherein the DXP pathway peptide is 1-deoxy-D-xylulose-5-phosphate synthase which belongs to the group EC 2.2.1.7.

14. The cells of claim 1, further comprising one or more introduced nucleic acids encoding MVA pathway polypeptide(s) having acetyl-CoA acetyltransferase, 3-hydroxy-3-methylglutaryl-CoA (HMG-CoA) reductase, 3-hydroxy-3-methylglutaryl-CoA (HMG-CoA) synthase, mevalonate kinase, phosphomevalonate kinase, and diphosphomevalonate decarboxylase activity.

15. The cells of claim 14, wherein the introduced nucleic acids comprise coding regions of mvaE and mvaS.

16. The cells of claim 1, wherein the heterologous nucleic acids are present on a plasmid comprising a gram positive origin of replication isolated from a plasmid isolated from Clostridium butyricum or from a plasmid isolated from Clostridium botulinum.

17. The cells of claim 1, comprising one or more genetic modifications resulting in the disruption or down regulation of the genes encoding one or more of the pathways for production of lactate, acetate, ethanol, succinate, or glycerol.

18. The cells of claim 1 wherein:
a) the cells are Clostridium ljungdahlii;
b) the cells are adapted for growth on syngas;
c) the isoprene synthase polypeptide is a truncated P. alba isoprene synthase; polypeptide having a his-tag at the N-terminus;
d) the isopentenyl diphosphate isomerase polypeptide is from S. cerevisiae;
e) the promoter is pFdx from C. sporogenes; and
f) the heterologous nucleic acids are in a plasmid having a gram positive origin of replication isolated from a plasmid isolated from Clostridium butyricum or Clostridium botulinum.

19. A method of producing isoprene comprising culturing cells of any of claims 1-18 under suitable conditions for the production of isoprene, wherein the culture conditions are substantially oxygen-free and comprise synthesis gas as energy and/or carbon source.

20. Recombinant obligate anaerobic bacterial acetogen cells comprising at least one heterologous nucleic acid encoding at least two polypeptides having acetyl-CoA acetyltransferase 3-hydroxy-3-methylglutaryl-CoA (HMG-CoA) reductase, and 3-hydroxy-3-methylglutaryl-CoA (HMG-CoA) synthase activities, wherein the heterologous nucleic acids are operably linked either separately or in an operon to a promoter that is active in the cells, and wherein the cells produce mevalonate when grown in substantially oxygen-free culture conditions comprising synthesis gas as energy and/or carbon source.

21. The cells of claim 20 wherein the polypeptides are encoded by mvaE and mvaS.

22. The cells of claim 21, wherein the cells are selected from the group consisting of Clostridium ljungdahlii, Clostridium aceticum, Moorella thermoacetica, Clostridium autoethanogenum, Eurobacterium limosum, Clostridium carboxydivorans, Peptostreptococcus productus, Rhodospirillum rubrum, Desulfitobacterium hafniense, Aecetoanaerobium notera, Butyribacterium methylotrophicum, Thermoanaerobacter kivui, Eubacterium limosum, Peptostreptococcus productus, and Acetobacterium woodi.

23. A method of producing mevalonate comprising culturing cells of any of claims 20-22 under suitable conditions for the production of mevalonate, wherein the culture conditions are substantially oxygen-free and comprise synthesis gas as energy and/or carbon source.

24. Recombinant obligate anaerobic bacterial acetogen cells capable of producing acetyl-CoA derived products, said cells comprising one or more heterologous nucleic acids encoding a polypeptide involved in the conversion of acetyl-CoA into a acetyl-CoA derived product in operable combination with a promoter, wherein the culturing of said cells under substantially oxygen-free culture conditions comprising synthesis gas as energy and/or carbon source provides for the production of said acetyl-CoA derived product.

25. The cells of claim 24, wherein the acetyl-CoA derived product is selected from the group consisting of 2-keto acids, malonyl-CoA, acetoacetyl-CoA and/or ethanol.

26. The cells of claim 25, further comprising: (a) one or more heterologous nucleic acids encoding a one or more polypeptides capable of converting a 2-keto acid into a non-fermentative alcohol; (b) one or more heterologous nucleic acids encoding one or more polypeptides capable of converting malonyl-CoA into a fatty acid-derived hydrocarbon; or (c) one or more heterologous nucleic acids encoding one or more polypeptides capable of converting acetoacetyl-CoA into a fermentative alcohol.

27. The cells of claim 26, wherein said non-fermentative alcohol is selected from the group consisting of 1-propanol, 1-butanol, isobutanol, 2-methyl-1-butanol, 3-methyl-1-butanol, 3-methyl-1-pentanol, 4-methyl-1-pentanol and 1-hexanol.

28. The cells of claim 27, wherein said fatty acid-derived hydrocarbon is selected from the group consisting of fatty alcohols, fatty esters, olefins, and alkanes.


SEQUENCE LISTING (62 sequences)

SEQ ID NO: 1 - DNA, 318 bp, Clostridium butyricum plasmid
atgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataa

SEQ ID NO: 2 - protein, 329 aa, Streptomyces sp. CL190
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 3 - protein, 418 aa, artificial sequence
  his tagged MvaS
MRGSHHHHHHGMASMTGGQQMGRDLYDDDDKDHPFTTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIVYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 4 - protein, 838 aa, artificial sequence
  his tagged MvaE
MRGSHHHHHHGMASMTGGQQMGRDLYDDDDKDHPFTKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADPESLIDKLQVREAEVFQQAELSYPSIVKRGGGLRDLQYRTFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRAMAILNDLRKQ

SEQ ID NO: 5 - protein, 309 aa, artificial sequence
  his tagged IDI
MGHHHHHHHHHHSSGHIEGRHMTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 6 - protein, 578 aa, artificial sequence
  his tagged IspS
MRGSHHHHHHGMASMTGGQQMGRDLYDDDDKDHPFTMRRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 7 - DNA, 2409 bp, Enterococcus faecalis
atgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcacatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgacgagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaa

SEQ ID NO: 8 - protein, 803 aa, Enterococcus faecalis
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADPESLIDKLQVREAEVFQQAELSYPSIVKRGGGLRDLQYRTFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRAMAILNDLRKQ

SEQ ID NO: 9 - DNA, 1149 bp, Enterococcus faecalis
atgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaac

SEQ ID NO: 10 - protein, 383 aa, Enterococcus faecalis
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEACYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIVYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 11 - DNA, 6590 bp, artificial sequence
  constructed plasmid
cctgcaggataaaaaaattgtagataaattttataaaatagttttatctacaatttttttatcaggaaacagctatgaccgcggccgcgtgtagtagcctgtgaaataagtaaggaaaaaaaagaagtaagtgttatatatgatgattattttgtagatgtagataggataatagaatccatagaaaatataggttatacagttatataaaaattactttaaaaattaataaaaacatggtaaaatataaatcgtataaagttgtgtaatttttaaggaggtgtgttacatatgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcacatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgacgagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataaaggaggtaaaaaaacatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaactaagctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccgaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtgtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccc

SEQ ID NO: 12 - DNA, 4476 bp, artificial sequence
  constructed plasmid
ggataaaaaaattgtagataaattttataaaatagttttatctacaatttttttatcaggaaacagctatgaccgcggccgctgtatccatatgaccatgattacgaattcgagctcggtacccggggatcctctagagtcgacgtcacgcgtccatggagatctcgaggcctgcagacatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccccctgca

SEQ ID NO: 13 - DNA, 25 bp, artificial sequence
  primer
ggcagtcacgcataacaaaggaatc

SEQ ID NO: 14 - DNA, 25 bp, artificial sequence
  primer
ggctgattgggttcaccgccatttg

SEQ ID NO: 15 - DNA, 25 bp, artificial sequence
  primer
aaaagacattccactatttctgaag

SEQ ID NO: 16 - DNA, 25 bp, artificial sequence
  primer
aaacatttccaaagattacttgatc

SEQ ID NO: 17 - DNA, 40 bp, artificial sequence
  primer
aggtttaaacttagggtaacaaaaaacaccgtatttctac

SEQ ID NO: 18 - DNA, 7224 bp, artificial sequence
  constructed plasmid
cctgcaggataaaaaaattgtagataaattttataaaatagttttatctacaatttttttatcaggaaacagctatgaccgcggccgcgtgtagtagcctgtgaaataagtaaggaaaaaaaagaagtaagtgttatatatgatgattattttgtagatgtagataggataatagaatccatagaaaatataggttatacagttatataaaaattactttaaaaattaataaaaacatggtaaaatataaatcgtataaagttgtgtaatttttaaggaggtgtgttacatatgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgttttaattacgaaacagaaagctacgatgcgcctttttctagtatgatgtatgatggattaacggatgcctttagtggtcaggcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtatcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtatcggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcacatgattgaaaatcaaatcagtgaaacagaagtgccgatgggcgttggcttacatttaacagtggacgaaactgattatttggtaccaatggcgacagaagagccctcagttattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaagtttttcaacaagcagagttaagttatccatctatcgttaaacggggcggcggcttaagagatttgcaatatcgtacttttgatgaatcatttgtatctgtcgactttttagtagatgttaaggatgcaatgggggcaaatatcgttaacgctatgttggaaggtgtggccgagttgttccgtgaatggtttgcggagcaaaagattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgacgagttggacgctggatggcgaacaactaattggtgaaatttcagttccgcttgctttagccacggttggcggtgccacaaaagtcttacctaaatctcaagcagctgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccatggctattttaaatgatttaagaaaacaataaaggaggtaaaaaaacatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggtcgtagcggcagatattgcaaaatatggcttaaattctggcggtgagcctacacaaggagctggggcggttgcaatgttagttgctagtgaaccgcgcattttggctttaaaagaggataatgtgatgctgacgcaagatatctatgacttttggcgtccaacaggccacccgtatcctatggtcgatggtcctttgtcaaacgaaacctacatccaatcttttgcccaagtctgggatgaacataaaaaacgaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagtatcgtctatagtcgtcgcgtaggaaacttgtatacgggttcactttatctgggactcatttcccttttagaaaatgcaacgactttaaccgcaggcaatcaaattggtttattcagttatggttctggtgctgtcgctgaatttttcactggtgaattagtagctggttatcaaaatcatttacaaaaagaaactcatttagcactgctggataatcggacagaactttctatcgctgaatatgaagccatgtttgcagaaactttagacacagacattgatcaaacgttagaagatgaattaaaatatagtatttctgctattaataataccgttcgttcttatcgaaactaagctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccc

SEQ ID NO: 19 - DNA, 25 bp, artificial sequence
  primer
cattaatgatggggcttctgctttg

SEQ ID NO: 20 - DNA, 25 bp, artificial sequence
  primer
aaacagaagtgccgatgggcgttgg

SEQ ID NO: 21 - DNA, 25 bp, artificial sequence
  primer
tttcatattggtattgggcaagacc

SEQ ID NO: 22 - DNA, 25 bp, artificial sequence
  primer
ctttagcgttccatattccttacac

SEQ ID NO: 23 - DNA, 6061 bp, artificial sequence
  constructed plasmid
cctgcaggataaaaaaattgtagataaattttataaaatagttttatctacaatttttttatcaggaaacagctatgaccgcggccgccataaattgaccttatataacaaataatgtataattgttttgaaatatggaggagaaaaatggaagcaagaagaagtgcaaactacgaacctaatagttgggattatgattatcttcttagtagtgatacagatgaaagtatagaagtatataaagataaagcaaaaaaacttgaagcagaagtaagaagagaaataaataacgaaaaagcagaatttcttacacttcttgaacttatagataatgtacaaagacttggacttggatatagatttgaaagtgatataagaggtgcacttgatagatttgtaagtagtggaggatttgatgcagtaacaaaaacatctcttcatggaacagctcttagttttagacttcttagacaacatggatttgaagtaagtcaagaagcattttctggatttaaagatcaaaatggaaattttcttgaaaatcttaaagaagatataaaagcaatacttagtctttatgaagcaagttttcttgcacttgaaggtgaaaatatacttgatgaagcaaaagtatttgcaataagtcatcttaaagaacttagtgaagaaaaaataggaaaagaacttgcagaacaagtaaatcatgcacttgaacttccacttcatagaagaacacaaagacttgaagcagtatggtctatagaagcatatagaaaaaaagaagatgcaaatcaagtacttcttgaacttgcaatacttgattataatatgatacaaagtgtatatcaaagagatcttagagaaacaagtagatggtggagaagagtaggacttgcaacaaaacttcattttgcaagagatagacttatagaaagtttttattgggcagttggagtagcatttgaacctcaatatagtgattgtagaaatagtgtagcaaaaatgtttagttttgtaacaataatagatgatatatacgatgtatatggaacacttgatgaacttgaactttttacagatgcagtagaaagatgggatgtaaatgcaataaatgatcttcctgattatatgaaactttgttttcttgctctttataatacaataaatgaaatagcatatgataatcttaaagataaaggtgaaaatattcttccttatcttacaaaagcatgggcagatctttgtaatgcatttcttcaagaggcaaaatggctttataataaaagtacacctacatttgatgattattttggaaatgcatggaaaagtagtagtggacctcttcaacttgtatttgcatattttgcagtagtacaaaatattaaaaaagaagaaatagaaaatcttcaaaaatatcatgatacaataagtagacctagtcatatatttagactttgtaatgatcttgcaagtgcaagtgcagaaatagcaagaggtgaaacagcaaatagtgtaagttgttatatgagaacaaaaggtataagtgaagaacttgcaacagaaagtgtaatgaatcttatagatgaaacatggaaaaaaatgaataaagaaaaacttggaggaagtctttttgcaaaaccttttgtagaaacagcaataaatcttgcaagacaaagtcattgtacatatcataatggtgatgcacatacaagtcctgatgagcttacaagaaaaagagtactttctgtaataacagaacctatacttccttttgaaagataggtgttataaggactagaggaaaatgaggagttgttatgaatgacagcagacaataattctatgccacatggagcagtatcttcatatgctaaattagttcagaaccaaacccctgaagacattttagaagaatttcctgaaattataccattacagcaaagacctaatacaagaagttctgaaacatcaaatgatgaatcaggtgaaacttgcttttctggacatgacgaagaacagattaaattaatgaatgaaaattgcattgtacttgattgggatgataatgctataggagctggtactaaaaaggtgtgtcatttaatggaaaacatagagaagggtttacttcatagagcattctcagttttcatatttaatgaacagggcgaacttttattacaacaaagagcaacagagaaaattactttccctgatttatggacaaatacttgttgcagtcacccattatgcatagatgatgagttgggtttgaaaggtaaattagatgataaaataaaaggtgctataaccgctgcagttagaaaattagatcacgagcttggcataccagaagatgagactaaaacaagaggtaaatttcacttcttgaataggattcactatatggctcctagtaatgagccttggggtgaacacgaaattgattacatactattttataaaataaacgctaaggagaatctaacagtaaacccaaatgtaaatgaggttagagactttaagtgggttagcccaaatgatcttaaaactatgtttgcagatccatcatataagtttacaccttggtttaaaataatttgcgaaaactacctttttaattggtgggagcagctagatgatttaagtgaagtagaaaatgataggcaaatacatagaatgctttaagctagcataaaaataagaagcctgcatttgcaggcttcttatttttatggcgcgccgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 24 - DNA, 106 bp, artificial sequence
  primer
tcgcggagctggtgaagtacatcaccgacgagcaaggcaagaccgatcgggccccctgcaggataaaaaaattgtagataaattttataaaatagttttatctaca

SEQ ID NO: 25 - DNA, 138 bp, artificial sequence
  primer
tgcacttcttcttgcttccatttttctcctccatatttcaaaacaattatacattatttgttatataangtcaatttatggcggccgcggtcatagctgtttcctgataaaaaaattgtagataaaactattttataa

SEQ ID NO: 26 - protein, 544 aa, artificial sequence
  P. alba Isps truncated
MEARRSANYEPNSWDYDYLLSSDTDESIEVYKDKAKKLEAEVRREINNEKAEFLTLLELIDNVQRLGLGYRFESDIRGALDRFVSSGGFDAVTKTSLHGTALSFRLLRQHGFEVSQEAFSGFKDQNGNFLENLKEDIKAILSLYEASFLALEGENILDEAKVFAISHLKELSEEKIGKELAEQVNHALELPLHRRTQRLEAVWSIEAYRKKEDANQVLLELAILDYNMIQSVYQRDLRETSRWWRRVGLATKLHFARDRLIESFYWAVGVAFEPQYSDCRNSVAKMFSFVTIIDDIYDVYGTLDELELFTDAVERWDVNAINDLPDYMKLCFLALYNTINEIAYDNLKDKGENILPYLTKAWADLCNAFLQEAKWLYNKSTPTFDDYFGNAWKSSSGPLQLVFAYFAVVQNIKKEEIENLQKYHDTISRPSHIFRLCNDLASASAEIARGETANSVSCYMRTKGISEELATESVMNLIDETWKKMNKEKLGGSLFAKPFVETAINLARQSHCTYHNGDAHTSPDELTRKRVLSVITEPILPFER

SEQ ID NO: 27 - DNA, 1632 bp, artificial sequence
  codon optimized coding region for P. alba IspS
atggaagcaagaagaagtgcaaactacgaacctaatagttgggattatgattatcttcttagtagtgatacagatgaaagtatagaagtatataaagataaagcaaaaaaacttgaagcagaagtaagaagagaaataaataacgaaaaagcagaatttcttacacttcttgaacttatagataatgtacaaagacttggacttggatatagatttgaaagtgatataagaggtgcacttgatagatttgtaagtagtggaggatttgatgcagtaacaaaaacatctcttcatggaacagctcttagttttagacttcttagacaacatggatttgaagtaagtcaagaagcattttctggatttaaagatcaaaatggaaattttcttgaaaatcttaaagaagatataaaagcaatacttagtctttatgaagcaagttttcttgcacttgaaggtgaaaatatacttgatgaagcaaaagtatttgcaataagtcatcttaaagaacttagtgaagaaaaaataggaaaagaacttgcagaacaagtaaatcatgcacttgaacttccacttcatagaagaacacaaagacttgaagcagtatggtctatagaagcatatagaaaaaaagaagatgcaaatcaagtacttcttgaacttgcaatacttgattataatatgatacaaagtgtatatcaaagagatcttagagaaacaagtagatggtggagaagagtaggacttgcaacaaaacttcattttgcaagagatagacttatagaaagtttttattgggcagttggagtagcatttgaacctcaatatagtgattgtagaaatagtgtagcaaaaatgtttagttttgtaacaataatagatgatatatacgatgtatatggaacacttgatgaacttgaactttttacagatgcagtagaaagatgggatgtaaatgcaataaatgatcttcctgattatatgaaactttgttttcttgctctttataatacaataaatgaaatagcatatgataatcttaaagataaaggtgaaaatattcttccttatcttacaaaagcatgggcagatctttgtaatgcatttcttcaagaggcaaaatggctttataataaaagtacacctacatttgatgattattttggaaatgcatggaaaagtagtagtggacctcttcaacttgtatttgcatattttgcagtagtacaaaatattaaaaaagaagaaatagaaaatcttcaaaaatatcatgatacaataagtagacctagtcatatatttagactttgtaatgatcttgcaagtgcaagtgcagaaatagcaagaggtgaaacagcaaatagtgtaagttgttatatgagaacaaaaggtataagtgaagaacttgcaacagaaagtgtaatgaatcttatagatgaaacatggaaaaaaatgaataaagaaaaacttggaggaagtctttttgcaaaaccttttgtagaaacagcaataaatcttgcaagacaaagtcattgtacatatcataatggtgatgcacatacaagtcctgatgagcttacaagaaaaagagtactttctgtaataacagaacctatacttccttttgaaaga

SEQ ID NO: 28 - DNA, 864 bp, artificial sequence
  condon optimized coding region for IDI
atgacagcagacaataattctatgccacatggagcagtatcttcatatgctaaattagttcagaaccaaacccctgaagacattttagaagaatttcctgaaattataccattacagcaaagacctaatacaagaagttctgaaacatcaaatgatgaatcaggtgaaacttgcttttctggacatgacgaagaacagattaaattaatgaatgaaaattgcattgtacttgattgggatgataatgctataggagctggtactaaaaaggtgtgtcatttaatggaaaacatagagaagggtttacttcatagagcattctcagttttcatatttaatgaacagggcgaacttttattacaacaaagagcaacagagaaaattactttccctgatttatggacaaatacttgttgcagtcacccattatgcatagatgatgagttgggtttgaaaggtaaattagatgataaaataaaaggtgctataaccgctgcagttagaaaattagatcacgagcttggcataccagaagatgagactaaaacaagaggtaaatttcacttcttgaataggattcactatatggctcctagtaatgagccttggggtgaacacgaaattgattacatactattttataaaataaacgctaaggagaatctaacagtaaacccaaatgtaaatgaggttagagactttaagtgggttagcccaaatgatcttaaaactatgtttgcagatccatcatataagtttacaccttggtttaaaataatttgcgaaaactacctttttaattggtgggagcagctagatgatttaagtgaagtagaaaatgataggcaaatacatagaatgctt

SEQ ID NO: 29 - protein, 288 aa, Saccharomyces cerevisiae
MTADNNSMPHGAVSSYAKLVQNQTPEDILEEFPEIIPLQQRPNTRSSETSNDESGETCFSGHDEEQIKLMNENCIVLDWDDNAIGAGTKKVCHLMENIEKGLLHRAFSVFIFNEQGELLLQQRATEKITFPDLWTNTCCSHPLCIDDELGLKGKLDDKIKGAITAAVRKLDHELGIPEDETKTRGKFHFLNRIHYMAPSNEPWGEHEIDYILFYKINAKENLTVNPNVNEVRDFKWVSPNDLKTMFADPSYKFTPWFKIICENYLFNWWEQLDDLSEVENDRQIHRML

SEQ ID NO: 30 - DNA, 20 bp, artificial sequence
  primer
atggaagcaagaagaagtgc

SEQ ID NO: 31 - DNA, 25 bp, artificial sequence
  primer
cctctagtccttataacacctatct

SEQ ID NO: 32 - DNA, 30 bp, artificial sequence
  primer
aggtgttataaggactagaggaaaatgagg

SEQ ID NO: 33 - DNA, 25 bp, artificial sequence
  primer
gcttgtagctaagtagtacgaaagg

SEQ ID NO: 34 - DNA, 25 bp, artificial sequence
  primer
atccttttgtatcggctcactacac

SEQ ID NO: 35 - DNA, 6808 bp, artificial sequence
  constructed plasmid
cctgcaggataaaaaaattgtagataaattttataaaatagttttatctacaatttttttatcaggaaacagctatgaccgcggccgccataaattgaccttatataacaaataatgtataattgttttgaaatatggaggagaaaaatggaagcaagaagaagtgcaaactacgaacctaatagttgggattatgattatcttcttagtagtgatacagatgaaagtatagaagtatataaagataaagcaaaaaaacttgaagcagaagtaagaagagaaataaataacgaaaaagcagaatttcttacacttcttgaacttatagataatgtacaaagacttggacttggatatagatttgaaagtgatataagaggtgcacttgatagatttgtaagtagtggaggatttgatgcagtaacaaaaacatctcttcatggaacagctcttagttttagacttcttagacaacatggatttgaagtaagtcaagaagcattttctggatttaaagatcaaaatggaaattttcttgaaaatcttaaagaagatataaaagcaatacttagtctttatgaagcaagttttcttgcacttgaaggtgaaaatatacttgatgaagcaaaagtatttgcaataagtcatcttaaagaacttagtgaagaaaaaataggaaaagaacttgcagaacaagtaaatcatgcacttgaacttccacttcatagaagaacacaaagacttgaagcagtatggtctatagaagcatatagaaaaaaagaagatgcaaatcaagtacttcttgaacttgcaatacttgattataatatgatacaaagtgtatatcaaagagatcttagagaaacaagtagatggtggagaagagtaggacttgcaacaaaacttcattttgcaagagatagacttatagaaagtttttattgggcagttggagtagcatttgaacctcaatatagtgattgtagaaatagtgtagcaaaaatgtttagttttgtaacaataatagatgatatatacgatgtatatggaacacttgatgaacttgaactttttacagatgcagtagaaagatgggatgtaaatgcaataaatgatcttcctgattatatgaaactttgttttcttgctctttataatacaataaatgaaatagcatatgataatcttaaagataaaggtgaaaatattcttccttatcttacaaaagcatgggcagatctttgtaatgcatttcttcaagaggcaaaatggctttataataaaagtacacctacatttgatgattattttggaaatgcatggaaaagtagtagtggacctcttcaacttgtatttgcatattttgcagtagtacaaaatattaaaaaagaagaaatagaaaatcttcaaaaatatcatgatacaataagtagacctagtcatatatttagactttgtaatgatcttgcaagtgcaagtgcagaaatagcaagaggtgaaacagcaaatagtgtaagttgttatatgagaacaaaaggtataagtgaagaacttgcaacagaaagtgtaatgaatcttatagatgaaacatggaaaaaaatgaataaagaaaaacttggaggaagtctttttgcaaaaccttttgtagaaacagcaataaatcttgcaagacaaagtcattgtacatatcataatggtgatgcacatacaagtcctgatgagcttacaagaaaaagagtactttctgtaataacagaacctatacttccttttgaaagataggtgttataaggactagaggaaaatgaggagttgttatgaatgacagcagacaataattctatgccacatggagcagtatcttcatatgctaaattagttcagaaccaaacccctgaagacattttagaagaatttcctgaaattataccattacagcaaagacctaatacaagaagttctgaaacatcaaatgatgaatcaggtgaaacttgcttttctggacatgacgaagaacagattaaattaatgaatgaaaattgcattgtacttgattgggatgataatgctataggagctggtactaaaaaggtgtgtcatttaatggaaaacatagagaagggtttacttcatagagcattctcagttttcatatttaatgaacagggcgaacttttattacaacaaagagcaacagagaaaattactttccctgatttatggacaaatacttgttgcagtcacccattatgcatagatgatgagttgggtttgaaaggtaaattagatgataaaataaaaggtgctataaccgctgcagttagaaaattagatcacgagcttggcataccagaagatgagactaaaacaagaggtaaatttcacttcttgaataggattcactatatggctcctagtaatgagccttggggtgaacacgaaattgattacatactattttataaaataaacgctaaggagaatctaacagtaaacccaaatgtaaatgaggttagagactttaagtgggttagcccaaatgatcttaaaactatgtttgcagatccatcatataagtttacaccttggtttaaaataatttgcgaaaactacctttttaattggtgggagcagctagatgatttaagtgaagtagaaaatgataggcaaatacatagaatgctttaagctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatcgggccc

SEQ ID NO: 36 - DNA, 27 bp, artificial sequence
  primer
gtcaaaaggcataacagtgctgaatag

SEQ ID NO: 37 - DNA, 31 bp, artificial sequence
  primer
atgtaacacacctccttaaaaattacacaac

SEQ ID NO: 38 - DNA, 55 bp, artificial sequence
  primer
taatttttaaggaggtgtgttacatatggaagcaagaagaagtgcaaactacgaa

SEQ ID NO: 39 - DNA, 31 bp, artificial sequence
  primer
attcagcactgttatgccttttgactatcac

SEQ ID NO: 40 - DNA, 24 bp, artificial sequence
  primer
gatgtagataggataatagaatcc

SEQ ID NO: 41 - DNA, 22 bp, artificial sequence
  primer
atcaggaaacagctatgaccgc

SEQ ID NO: 42 - DNA, 24 bp, artificial sequence
  primer
gatttgaaagtgatataagaggtg

SEQ ID NO: 43 - DNA, 23 bp, artificial sequence
  primer
gaacttgaactttttacagatgc

SEQ ID NO: 44 - DNA, 23 bp, artificial sequence
  primer
tgaatcttatagatgaaacatgg

SEQ ID NO: 45 - DNA, 22 bp, artificial sequence
  primer
agaacacaaagacttgaagcag

SEQ ID NO: 46 - DNA, 24 bp, artificial sequence
  primer
caggacttgtatgtgcatcaccat

SEQ ID NO: 47 - DNA, 24 bp, artificial sequence
  primer
gatcatttattgcatttacatccc

SEQ ID NO: 48 - DNA, 23 bp, artificial sequence
  primer
tgaagagatgtttttgttactgc

SEQ ID NO: 49 - DNA, 24 bp, artificial sequence
  primer
gacagcagacaataattctatgcc

SEQ ID NO: 50 - DNA, 24 bp, artificial sequence
  primer
taaggagaatctaacagtaaaccc

SEQ ID NO: 51 - DNA, 23 bp, artificial sequence
  primer
gaaaattagatcacgagcttggc

SEQ ID NO: 52 - DNA, 24 bp, artificial sequence
  primer
cacttaaatcatctagctgctccc

SEQ ID NO: 53 - DNA, 25 bp, artificial sequence
  primer
ctctttgttgtaataaaagttcgcc

SEQ ID NO: 54 - DNA, 25 bp, artificial sequence
  primer
tgtacgttcttttttctgttcttcc

SEQ ID NO: 55 - DNA, 6186 bp, artificial sequence
  constructed plasmid
cctgcaggataaaaaaattgtagataaattttataaaatagttttatctacaatttttttatcaggaaacagctatgaccgcggccgcgtgtagtagcctgtgaaataagtaaggaaaaaaaagaagtaagtgttatatatgatgattattttgtagatgtagataggataatagaatccatagaaaatataggttatacagttatataaaaattactttaaaaattaataaaaacatggtaaaatataaatcgtataaagttgtgtaatttttaaggaggtgtgttacatatggaagcaagaagaagtgcaaactacgaacctaatagttgggattatgattatcttcttagtagtgatacagatgaaagtatagaagtatataaagataaagcaaaaaaacttgaagcagaagtaagaagagaaataaataacgaaaaagcagaatttcttacacttcttgaacttatagataatgtacaaagacttggacttggatatagatttgaaagtgatataagaggtgcacttgatagatttgtaagtagtggaggatttgatgcagtaacaaaaacatctcttcatggaacagctcttagttttagacttcttagacaacatggatttgaagtaagtcaagaagcattttctggatttaaagatcaaaatggaaattttcttgaaaatcttaaagaagatataaaagcaatacttagtctttatgaagcaagttttcttgcacttgaaggtgaaaatatacttgatgaagcaaaagtatttgcaataagtcatcttaaagaacttagtgaagaaaaaataggaaaagaacttgcagaacaagtaaatcatgcacttgaacttccacttcatagaagaacacaaagacttgaagcagtatggtctatagaagcatatagaaaaaaagaagatgcaaatcaagtacttcttgaacttgcaatacttgattataatatgatacaaagtgtatatcaaagagatcttagagaaacaagtagatggtggagaagagtaggacttgcaacaaaacttcattttgcaagagatagacttatagaaagtttttattgggcagttggagtagcatttgaacctcaatatagtgattgtagaaatagtgtagcaaaaatgtttagttttgtaacaataatagatgatatatacgatgtatatggaacacttgatgaacttgaactttttacagatgcagtagaaagatgggatgtaaatgcaataaatgatcttcctgattatatgaaactttgttttcttgctctttataatacaataaatgaaatagcatatgataatcttaaagataaaggtgaaaatattcttccttatcttacaaaagcatgggcagatctttgtaatgcatttcttcaagaggcaaaatggctttataataaaagtacacctacatttgatgattattttggaaatgcatggaaaagtagtagtggacctcttcaacttgtatttgcatattttgcagtagtacaaaatattaaaaaagaagaaatagaaaatcttcaaaaatatcatgatacaataagtagacctagtcatatatttagactttgtaatgatcttgcaagtgcaagtgcagaaatagcaagaggtgaaacagcaaatagtgtaagttgttatatgagaacaaaaggtataagtgaagaacttgcaacagaaagtgtaatgaatcttatagatgaaacatggaaaaaaatgaataaagaaaaacttggaggaagtctttttgcaaaaccttttgtagaaacagcaataaatcttgcaagacaaagtcattgtacatatcataatggtgatgcacatacaagtcctgatgagcttacaagaaaaagagtactttctgtaataacagaacctatacttccttttgaaagataggtgttataaggactagaggaaaatgaggagttgttatgaatgacagcagacaataattctatgccacatggagcagtatcttcatatgctaaattagttcagaaccaaacccctgaagacattttagaagaatttcctgaaattataccattacagcaaagacctaatacaagaagttctgaaacatcaaatgatgaatcaggtgaaacttgcttttctggacatgacgaagaacagattaaattaatgaatgaaaattgcattgtacttgattgggatgataatgctataggagctggtactaaaaaggtgtgtcatttaatggaaaacatagagaagggtttacttcatagagcattctcagttttcatatttaatgaacagggcgaacttttattacaacaaagagcaacagagaaaattactttccctgatttatggacaaatacttgttgcagtcacccattatgcatagatgatgagttgggtttgaaaggtaaattagatgataaaataaaaggtgctataaccgctgcagttagaaaattagatcacgagcttggcataccagaagatgagactaaaacaagaggtaaatttcacttcttgaataggattcactatatggctcctagtaatgagccttggggtgaacacgaaattgattacatactattttataaaataaacgctaaggagaatctaacagtaaacccaaatgtaaatgaggttagagactttaagtgggttagcccaaatgatcttaaaactatgtttgcagatccatcatataagtttacaccttggtttaaaataatttgcgaaaactacctttttaattggtgggagcagctagatgatttaagtgaagtagaaaatgataggcaaatacatagaatgctttaagctagcataaaaataagaagcctgcatttgcaggcttcttatttttatggcgcgccgccattatttttttgaacaattgacaattcatttcttattttttattaagtgatagtcaaaaggcataacagtgctgaatagaaagaaatttacagaaaagaaaattatagaatttagtatgattaattatactcatttatgaatgtttaattgaatacaaaaaaaaatacttgttatgtattcaattacgggttaaaatatagacaagttgaaaaatttaataaaaaaataagtcctcagctcttatatattaagctaccaacttagtatataagccaaaacttaaatgtgctaccaacacatcaagccgttagagaactctatctatagcaatatttcaaatgtaccgacatacaagagaaacattaactatatatattcaatttatgagattatcttaacagatataaatgtaaattgcaataagtaagatttagaagtttatagcctttgtgtattggaagcagtacgcaaaggcttttttatttgataaaaattagaagtatatttattttttcataattaatttatgaaaatgaaagggggtgagcaaagtgacagaggaaagcagtatcttatcaaataacaaggtattagcaatatcattattgactttagcagtaaacattatgacttttatagtgcttgtagctaagtagtacgaaagggggagctttaaaaagctccttggaatacatagaattcataaattaatttatgaaaagaagggcgtatatgaaaacttgtaaaaattgcaaagagtttattaaagatactgaaatatgcaaaatacattcgttgatgattcatgataaaacagtagcaacctattgcagtaaatacaatgagtcaagatgtttacataaagggaaagtccaatgtattaattgttcaaagatgaaccgatatggatggtgtgccataaaaatgagatgttttacagaggaagaacagaaaaaagaacgtacatgcattaaatattatgcaaggagctttaaaaaagctcatgtaaagaagagtaaaaagaaaaaataatttatttattaatttaatattgagagtgccgacacagtatgcactaaaaaatatatctgtggtgtagtgagccgatacaaaaggatagtcactcgcattttcataatacatcttatgttatgattatgtgtcggtgggacttcacgacgaaaacccacaataaaaaaagagttcggggtagggttaagcatagttgaggcaactaaacaatcaagctaggatatgcagtagcagaccgtaaggtcgttgtttaggtgtgttgtaatacatacgctattaagatgtaaaaatacggataccaatgaagggaaaagtataatttttggatgtagtttgtttgttcatctatgggcaaactacgtccaaagccgtttccaaatctgctaaaaagtatatcctttctaaaatcaaagtcaagtatgaaatcataaataaagtttaattttgaagttattatgatattatgtttttctattaaaataaattaagtatatagaatagtttaataatagtatatacttaatgtgataagtgtctgacagtgtcacagaaaggatgattgttatggattataagcggccggccagtgggcaagttgaaaaattcacaaaaatgtggtataatatctttgttcattagagcgataaacttgaatttgagagggaacttagatggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaacttcaggtttgtctgtaactaaaaacaagtatttaagcaaaaacatcgtagaaatacggtgttttttgttaccctaagtttaaactcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcagggccc

SEQ ID NO: 56 - DNA, 61 bp, artificial sequence
  primer
ttaaggaggtgtgttacatatgcatcaccatcaccatcacgaagcaagaagaagtgcaaac

SEQ ID NO: 57 - DNA, 61 bp, artificial sequence
  primer
gtttgcacttcttcttgcttcgtgatggtgatggtgatgcatatgtaacacacctccttaa

SEQ ID NO: 58 - DNA, 61 bp, artificial sequence
  primer
cctatacttccttttgaaagacatcaccatcaccatcactaggtgttataaggactagagg

SEQ ID NO: 59 - DNA, 61 bp, artificial sequence
  primer
cctctagtccttataacacctagtgatggtgatggtgatgtctttcaaaaggaagtatagg

SEQ ID NO: 60 - DNA, 24 bp, artificial sequence
  primer
gatgtagataggataatagaatcc

SEQ ID NO: 61 - DNA, 25 bp, artificial sequence
  primer
ctctttgttgtaataaaagttcgcc

SEQ ID NO: 62 - DNA, 1056 bp, Clostridium botulinum plasmid
atgactaattataatcaaaaaaatgaaaataaacaagaggtaaaaactgctttagagaaatgtactgataaaaaaagaaaaaatcctagatttacgtcatacatagcacctttaactactaagaaaaatattgaaaggacttccacttgtggagattatttgtttatgttgagtgatgcagacttagaacattttaaattacataaaggtaatttttgcggtaatagattttgtccaatgtgtagttggcgacttgcttgtaaggatagtttagaaatatctattcttatggagcatttaagaaaagaagaaaataaagagtttatatttttaactcttacaactccaaatgtaaaaagttatgatcttaattattctattaaacaatataataaatcttttaaaaaattaatggagcgtaaggaagttaaggatataactaaaggttatataagaaaattagaagtaacttaccaaaaggaaaaatacataacaaaggatttatggaaaataaaaaaagattattatcaaaaaaaaggacttgaaattggtgatttagaacctaattttgatacttataatcctcattttcatgtagttattgcagttaataaaagttattttacagataaaaattattatataaatcgagaaagatggttggaattatggaagtttgctactaaggatgattctataactcaagttgatgttagaaaagcaaaaattaatgattataaagaggtttacgaacttgcgaaatattcagctaaagacactgattatttaatatcgaggccagtatttgaaattttttataaagcattaaaaggcaagcaggtattagtttttagtggattttttaaagatgcacacaaattgtacaagcaaggaaaacttgatgtttataaaaagaaagatgaaattaaatatgtctatatagtttattataattggtgcaaaaaacaatatgaaaaaactagaataagggaacttacggaagatgaaaaagaagaattaaatcaagatttaatagatgaaatagaaatagattaa